SEQUENCE LISTING (197 sequences)

SEQ ID NO: 1 - DNA, 139 bp, Artificial Sequence
  Secretion Signal nucleic acid sequence
atgggatggagctgtatcatcctcttcttggtagcaacagctacaggtaaggggttaacagtagcaggcttgaggtctggacatatatatgggtgacaatgacatccactttgcctttctctccacaggcgcgcacagt

SEQ ID NO: 2 - protein, 19 aa, Artificial Sequence
  Secretion Signal amino acid sequence
MGWSCIILFLVATATGAHS

SEQ ID NO: 3 - DNA, 27 bp, Influenza A virus
ctgtaccagaacgtgggcacctacgtg

SEQ ID NO: 4 - DNA, 27 bp, Influenza A virus
atctacagcaccgtggccagcagcctg

SEQ ID NO: 5 - DNA, 27 bp, Influenza A virus
acctaccagcggaccagagccctcgtg

SEQ ID NO: 6 - DNA, 24 bp, Mus musculus
tacgccatgatctaccggaacctg

SEQ ID NO: 7 - DNA, 24 bp, Mus musculus
aggacttatacctacgaaaagctc

SEQ ID NO: 8 - DNA, 24 bp, Mus musculus
tcaggatacgacttcagtcgcctc

SEQ ID NO: 9 - protein, 9 aa, Influenza A virus
LYQNVGTYV

SEQ ID NO: 10 - protein, 9 aa, Influenza A virus
IYSTVASSL

SEQ ID NO: 11 - protein, 9 aa, Influenza A virus
TYQRTRALV

SEQ ID NO: 12 - protein, 8 aa, Mus musculus
YAMIYRNL

SEQ ID NO: 13 - protein, 8 aa, Mus musculus
RTYTYEKL

SEQ ID NO: 14 - protein, 8 aa, Mus musculus
SGYDFSRL

SEQ ID NO: 15 - DNA, 45 bp, Artificial Sequence
  Linker 15aa Long nucleic acid sequence
ggcggcggaggatctggcggaggtggaagtgggggaggcggcagc

SEQ ID NO: 16 - protein, 15 aa, Artificial Sequence
  Linker 15aa Long amino acid sequence
GGGGSGGGGSGGGGS

SEQ ID NO: 17 - DNA, 60 bp, Artificial Sequence
  Linker 20aa long nucleic acid sequence
ggcggagggggtagcggaggcggtggttctggcggaggtggctcagggggagggggatct

SEQ ID NO: 18 - protein, 20 aa, Artificial Sequence
  Linker 20aa long amino acid sequence
GGGGSGGGGSGGGGSGGGGS

SEQ ID NO: 19 - DNA, 297 bp, Mus musculus
atccagaaaaccccccagatccaggtgtacagccggcacccccccgagaacggcaagcccaacatcctgaactgctacgtgacccagttccacccccctcacatcgagatccagatgctgaagaatggcaagaagatccccaaggtcgagatgagcgacatgagcttcagcaaggactggtccttctacatcctggcccacaccgagttcacccccaccgaaaccgacacctacgcctgcagagtgaagcacgtgtccatggccgagcccaagaccgtgtactgggaccgggatatg

SEQ ID NO: 20 - protein, 99 aa, Mus musculus
IQKTPQIQVYSRHPPENGKPNILNCYVTQFHPPHIEIQMLKNGKKIPKVEMSDMSFSKDWSFYILAHTEFTPTETDTYACRVKHVSMAEPKTVYWDRDM

SEQ ID NO: 21 - DNA, 840 bp, Mus musculus
ggcccccacagcctgcggtacttcgtgaccgccgtgtccagacctggcctgggcgagccccggtacatggaagtgggctacgtggacgacaccgagttcgtcagattcgacagcgacgccgagaaccccagatacgagcccagagcccggtggatggaacaggaaggccccgagtactgggagagagagacacagaaggccaagggcaacgagcagagcttccgggtggacctgcggaccctgctgggctactacaaccagagcaagggcggcagccacaccatccaggtcatcagcggctgcgaagtgggaagcgacggccggctgctgcggggctaccagcagtacgcctacgatggctgcgactatatcgccctgaacgaggacctgaaaacctggacagccgccgacatggccgccctgatcaccaagcacaagtgggagcaggccggggaggccgagagactgagagcctacctggaaggcacctgtgtggaatggctgagaagatacctgaagaacggcaacgccacactgctgagaaccgacagccccaaggcccacgtgacccaccacagcagacccgaggacaaagtgaccctgcggtgctgggccctgggcttctaccccgccgatatcaccctgacctggcagctgaacggcgaggaactgatccaggacatggaactggtggaaacccggcctgccggcgacggcaccttccagaaatgggccagcgtggtggtgcccctgggaaaagagcagtactacacctgtcacgtgtaccaccagggcctgcccgagcccctgaccctgagatgggagcctccacctagcacc

SEQ ID NO: 22 - protein, 280 aa, Mus musculus
GPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPST

SEQ ID NO: 23 - DNA, 840 bp, Mus musculus
ggcccccacagcctgcggtacttcgtgaccgccgtgtccagacctggcctgggcgagcctagattcattgccgtgggctacgtggacgacacccagttcgtcagattcgacagcgacgccgacaacccccgcttcgagcccagagccccctggatggaacaggaaggccccgagtactgggaggaacagacccagcgggccaagagcgacgagcagtggttccgggtgtccctgcggaccgcccagcggtactacaaccagagcaagggcggcagccacaccttccagcggatgttcggctgcgacgtgggcagcgactggcggctgctgagaggctaccagcagttcgcctacgacggccgggactatatcgccctgaacgaggacctgaaaacctggacagccgccgacactgccgccctgatcaccagacggaagtgggaacaggccggcgacgctgagtactaccgggcctacctggaaggcgagtgcgtggaatggctgcggagatatctggaactgggcaacgagacactgctgaggaccgacagccccaaggcccacgtgacctaccaccccagatcccaggtggacgtgaccctgagatgctgggccctgggcttctaccccgccgatatcaccctgacctggcagctgaacggcgaggatctgacccaggacatggaactcgtggaaacccggcctgccggggacggcacctttcagaaatgggccgctgtggtggtgcccctgggcaaagagcagaactacacctgtcacgtgcaccacaagggcctgcccgagcctctgaccctgcggtggaagctgcctcctagcacc

SEQ ID NO: 24 - protein, 280 aa, Mus musculus
GPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRWKLPPST

SEQ ID NO: 25 - DNA, 12 bp, Artificial Sequence
  Short Connector DNA sequence
gccagcggcgga

SEQ ID NO: 26 - DNA, 738 bp, Artificial Sequence
  Anti MCSP scFv of 225.28S DNA sequence
caggtcaaactgcagcagagcggcggaggcctggtgcagcctggcggcagcatgaagctgagctgcgtggtgtccggcttcaccttcagcaactactggatgaactgggtccgacagagccccgagaagggcctggaatggatcgccgagatccggctgaagtccaacaacttcggccggtactacgccgagagcgtgaagggcagattcaccatcagccgggacgacagcaagagcagcgcctacctgcagatgatcaacctgcgggccgaggacaccggcatctactactgcaccagctacggcaactacgtgggccactacttcgaccactggggccagggcaccaccgtgaccgtgtctagcggaggcggaggatctggcggaggtggaagtggcgggggaggcagcgatatcgagctgacccagtcccccaagttcatgagcaccagcgtgggcgaccgggtgtccgtgacatgcaaggccagccagaacgtggacaccaacgtggcctggtatcagcagaagcccggccagagccctgagcccctgctgttcagcgccagctacagatacaccggcgtgcccgacaggttcaccggcagcggctctggcaccgacttcaccctgaccatctccaacgtgcagagcgaggacctggccgagtacttctgccagcagtacaacagctaccccctgacctttggaggcggcaccaagctggaaatcaagcgggcc

SEQ ID NO: 27 - protein, 246 aa, Artificial Sequence
  Anti MCSP scFv of 225.28S amino acid sequence
QVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSSGGGGSGGGGSGGGGSDIELTQSPKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIKRA

SEQ ID NO: 28 - DNA, 18 bp, Artificial Sequence
  His Tag nucleic acid sequence
catcatcaccatcaccat

SEQ ID NO: 29 - protein, 6 aa, Artificial Sequence
  His Tag amino acid sequence
HHHHHH

SEQ ID NO: 30 - DNA, 45 bp, Artificial Sequence
  BirA Tag DNA sequence
ggacttaatgatatcttcgaggcgcagaagattgaatggcactga

SEQ ID NO: 31 - protein, 14 aa, Artificial Sequence
  BirA Tag amino acid sequence
GLNDIFEAQKIEWH

SEQ ID NO: 32 - DNA, 1480 bp, Artificial Sequence
  CG DNA of Soluble single chain MHC H2Kb molecule with YAMIYRNL peptide with Tags, without the scFv nucleic acid sequence
atgggatggagctgtatcatcctcttcttggtagcaacagctacaggtaaggggttaacagtagcaggcttgaggtctggacatatatatgggtgacaatgacatccactttgcctttctctccacaggcgcgcacagttacgccatgatctaccggaacctgggcggcggaggatctggcggaggtggaagtgggggaggcggcagcatccagaaaaccccccagatccaggtgtacagccggcacccccccgagaacggcaagcccaacatcctgaactgctacgtgacccagttccacccccctcacatcgagatccagatgctgaagaatggcaagaagatccccaaggtcgagatgagcgacatgagcttcagcaaggactggtccttctacatcctggcccacaccgagttcacccccaccgaaaccgacacctacgcctgcagagtgaagcacgtgtccatggccgagcccaagaccgtgtactgggaccgggatatgggcggagggggtagcggaggcggtggttctggcggaggtggctcagggggagggggatctggcccccacagcctgcggtacttcgtgaccgccgtgtccagacctggcctgggcgagccccggtacatggaagtgggctacgtggacgacaccgagttcgtcagattcgacagcgacgccgagaaccccagatacgagcccagagcccggtggatggaacaggaaggccccgagtactgggagagagagacacagaaggccaagggcaacgagcagagcttccgggtggacctgcggaccctgctgggctactacaaccagagcaagggcggcagccacaccatccaggtcatcagcggctgcgaagtgggaagcgacggccggctgctgcggggctaccagcagtacgcctacgatggctgcgactatatcgccctgaacgaggacctgaaaacctggacagccgccgacatggccgccctgatcaccaagcacaagtgggagcaggccggggaggccgagagactgagagcctacctggaaggcacctgtgtggaatggctgagaagatacctgaagaacggcaacgccacactgctgagaaccgacagccccaaggcccacgtgacccaccacagcagacccgaggacaaagtgaccctgcggtgctgggccctgggcttctaccccgccgatatcaccctgacctggcagctgaacggcgaggaactgatccaggacatggaactggtggaaacccggcctgccggcgacggcaccttccagaaatgggccagcgtggtggtgcccctgggaaaagagcagtactacacctgtcacgtgtaccaccagggcctgcccgagcccctgaccctgagatgggagcctccacctagcaccgccagcggcggacatcatcaccatcaccatggacttaatgatatcttcgaggcgcagaagattgaatggcactga

SEQ ID NO: 33 - protein, 465 aa, Artificial Sequence
  CG Protein Soluble fusion molecule H2Kb molecule with YAMIYRNL peptide with Tags, without the scFv amino acid sequence
MGWSCIILFLVATATGAHSYAMIYRNLGGGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHVSMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTASGGHHHHHHGLNDIFEAQKIEWH

SEQ ID NO: 34 - DNA, 2218 bp, Artificial Sequence
  BA DNA of Soluble single chain MHC H2Kb molecule with YAMIYRNL peptide anti MCSP scFv of 225.28S clone and tags nucleic acid sequence
atgggatggagctgtatcatcctcttcttggtagcaacagctacaggtaaggggttaacagtagcaggcttgaggtctggacatatatatgggtgacaatgacatccactttgcctttctctccacaggcgcgcacagttacgccatgatctaccggaacctgggcggcggaggatctggcggaggtggaagtgggggaggcggcagcatccagaaaaccccccagatccaggtgtacagccggcacccccccgagaacggcaagcccaacatcctgaactgctacgtgacccagttccacccccctcacatcgagatccagatgctgaagaatggcaagaagatccccaaggtcgagatgagcgacatgagcttcagcaaggactggtccttctacatcctggcccacaccgagttcacccccaccgaaaccgacacctacgcctgcagagtgaagcacgtgtccatggccgagcccaagaccgtgtactgggaccgggatatgggcggagggggtagcggaggcggtggttctggcggaggtggctcagggggagggggatctggcccccacagcctgcggtacttcgtgaccgccgtgtccagacctggcctgggcgagccccggtacatggaagtgggctacgtggacgacaccgagttcgtcagattcgacagcgacgccgagaaccccagatacgagcccagagcccggtggatggaacaggaaggccccgagtactgggagagagagacacagaaggccaagggcaacgagcagagcttccgggtggacctgcggaccctgctgggctactacaaccagagcaagggcggcagccacaccatccaggtcatcagcggctgcgaagtgggaagcgacggccggctgctgcggggctaccagcagtacgcctacgatggctgcgactatatcgccctgaacgaggacctgaaaacctggacagccgccgacatggccgccctgatcaccaagcacaagtgggagcaggccggggaggccgagagactgagagcctacctggaaggcacctgtgtggaatggctgagaagatacctgaagaacggcaacgccacactgctgagaaccgacagccccaaggcccacgtgacccaccacagcagacccgaggacaaagtgaccctgcggtgctgggccctgggcttctaccccgccgatatcaccctgacctggcagctgaacggcgaggaactgatccaggacatggaactggtggaaacccggcctgccggcgacggcaccttccagaaatgggccagcgtggtggtgcccctgggaaaagagcagtactacacctgtcacgtgtaccaccagggcctgcccgagcccctgaccctgagatgggagcctccacctagcaccgccagcggcggacaggtcaaactgcagcagagcggcggaggcctggtgcagcctggcggcagcatgaagctgagctgcgtggtgtccggcttcaccttcagcaactactggatgaactgggtccgacagagccccgagaagggcctggaatggatcgccgagatccggctgaagtccaacaacttcggccggtactacgccgagagcgtgaagggcagattcaccatcagccgggacgacagcaagagcagcgcctacctgcagatgatcaacctgcgggccgaggacaccggcatctactactgcaccagctacggcaactacgtgggccactacttcgaccactggggccagggcaccaccgtgaccgtgtctagcggaggcggaggatctggcggaggtggaagtggcgggggaggcagcgatatcgagctgacccagtcccccaagttcatgagcaccagcgtgggcgaccgggtgtccgtgacatgcaaggccagccagaacgtggacaccaacgtggcctggtatcagcagaagcccggccagagccctgagcccctgctgttcagcgccagctacagatacaccggcgtgcccgacaggttcaccggcagcggctctggcaccgacttcaccctgaccatctccaacgtgcagagcgaggacctggccgagtacttctgccagcagtacaacagctaccccctgacctttggaggcggcaccaagctggaaatcaagcgggcccatcatcaccatcaccatggacttaatgatatcttcgaggcgcagaagattgaatggcactga

SEQ ID NO: 35 - protein, 711 aa, Artificial Sequence
  BA Protein Soluble fusion molecule H2Kb molecule with YAMIYRNL peptide anti MCSP scFv of 225.28S clone and tags amino acid sequence
MGWSCIILFLVATATGAHSYAMIYRNLGGGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHVSMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTASGGQVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSSGGGGSGGGGSGGGGSDIELTQSPKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIKRAHHHHHHGLNDIFEAQKIEWH

SEQ ID NO: 36 - DNA, 2176 bp, Artificial Sequence
  M15 DNA of Soluble single chain MHC H2Kb molecule with YAMIYRNL peptide anti MCSP scFv of 225.28S clone and tags nucleic acid sequence
atgggatggagctgtatcatcctcttcttggtagcaacagctacaggtaaggggttaacagtagcaggcttgaggtctggacatatatatgggtgacaatgacatccactttgcctttctctccacaggcgcgcacagttacgccatgatctaccggaacctgggcggcggaggatctggcggaggtggaagtgggggaggcggcagcatccagaaaaccccccagatccaggtgtacagccggcacccccccgagaacggcaagcccaacatcctgaactgctacgtgacccagttccacccccctcacatcgagatccagatgctgaagaatggcaagaagatccccaaggtcgagatgagcgacatgagcttcagcaaggactggtccttctacatcctggcccacaccgagttcacccccaccgaaaccgacacctacgcctgcagagtgaagcacgtgtccatggccgagcccaagaccgtgtactgggaccgggatatgggcggagggggtagcggaggcggtggttctggcggaggtggctcagggggagggggatctggcccccacagcctgcggtacttcgtgaccgccgtgtccagacctggcctgggcgagccccggtacatggaagtgggctacgtggacgacaccgagttcgtcagattcgacagcgacgccgagaaccccagatacgagcccagagcccggtggatggaacaggaaggccccgagtactgggagagagagacacagaaggccaagggcaacgagcagagcttccgggtggacctgcggaccctgctgggctactacaaccagagcaagggcggcagccacaccatccaggtcatcagcggctgcgaagtgggaagcgacggccggctgctgcggggctaccagcagtacgcctacgatggctgcgactatatcgccctgaacgaggacctgaaaacctggacagccgccgacatggccgccctgatcaccaagcacaagtgggagcaggccggggaggccgagagactgagagcctacctggaaggcacctgtgtggaatggctgagaagatacctgaagaacggcaacgccacactgctgagaaccgacagccccaaggcccacgtgacccaccacagcagacccgaggacaaagtgaccctgcggtgctgggccctgggcttctaccccgccgatatcaccctgacctggcagctgaacggcgaggaactgatccaggacatggaactggtggaaacccggcctgccggcgacggcaccttccagaaatgggccagcgtggtggtgcccctgggaaaagagcagtactacacctgtcacgtgtaccaccagggcctgcccgagcccctgaccctgagatgggagcctccacctagcaccgccagcggcggacaggtcaaactgcagcagagcggcggaggcctggtgcagcctggcggcagcatgaagctgagctgcgtggtgtccggcttcaccttcagcaactactggatgaactgggtccgacagagccccgagaagggcctggaatggatcgccgagatccggctgaagtccaacaacttcggccggtactacgccgagagcgtgaagggcagattcaccatcagccgggacgacagcaagagcagcgcctacctgcagatgatcaacctgcgggccgaggacaccggcatctactactgcaccagctacggcaactacgtgggccactacttcgaccactggggccagggcaccaccgtgaccgtgtctagcggaggcggaggatctggcggaggtggaagtggcgggggaggcagcgatatcgagctgacccagtcccccaagttcatgagcaccagcgtgggcgaccgggtgtccgtgacatgcaaggccagccagaacgtggacaccaacgtggcctggtatcagcagaagcccggccagagccctgagcccctgctgttcagcgccagctacagatacaccggcgtgcccgacaggttcaccggcagcggctctggcaccgacttcaccctgaccatctccaacgtgcagagcgaggacctggccgagtacttctgccagcagtacaacagctaccccctgacctttggaggcggcaccaagctggaaatcaagcgggcccatcatcaccatcaccattga

SEQ ID NO: 37 - protein, 697 aa, Artificial Sequence
  M15 Soluble fusion molecule H2Kb molecule with YAMIYRNL peptide anti MCSP scFv of 225.28S clone and tags amino acid sequence
MGWSCIILFLVATATGAHSYAMIYRNLGGGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHVSMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTASGGQVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSSGGGGSGGGGSGGGGSDIELTQSPKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIKRAHHHHHH

SEQ ID NO: 38 - DNA, 1483 bp, Artificial Sequence
  CG DNA of Soluble single chain MHC H2Kd molecule with TYQRTRALV peptide with Tags, without the scFv nu cleic acid sequence
atgggatggagctgtatcatcctcttcttggtagcaacagctacaggtaaggggttaacagtagcaggcttgaggtctggacatatatatgggtgacaatgacatccactttgcctttctctccacaggcgcgcacagtacctaccagcggaccagagccctcgtgggcggcggaggatctggcggaggtggaagtgggggaggcggcagcatccagaaaaccccccagatccaggtgtacagccggcacccccccgagaacggcaagcccaacatcctgaactgctacgtgacccagttccacccccctcacatcgagatccagatgctgaagaatggcaagaagatccccaaggtcgagatgagcgacatgagcttcagcaaggactggtccttctacatcctggcccacaccgagttcacccccaccgaaaccgacacctacgcctgcagagtgaagcacgtgtccatggccgagcccaagaccgtgtactgggaccgggatatgggcggagggggtagcggaggcggtggttctggcggaggtggctcagggggagggggatctggcccccacagcctgcggtacttcgtgaccgccgtgtccagacctggcctgggcgagcctagattcattgccgtgggctacgtggacgacacccagttcgtcagattcgacagcgacgccgacaacccccgcttcgagcccagagccccctggatggaacaggaaggccccgagtactgggaggaacagacccagcgggccaagagcgacgagcagtggttccgggtgtccctgcggaccgcccagcggtactacaaccagagcaagggcggcagccacaccttccagcggatgttcggctgcgacgtgggcagcgactggcggctgctgagaggctaccagcagttcgcctacgacggccgggactatatcgccctgaacgaggacctgaaaacctggacagccgccgacactgccgccctgatcaccagacggaagtgggaacaggccggcgacgctgagtactaccgggcctacctggaaggcgagtgcgtggaatggctgcggagatatctggaactgggcaacgagacactgctgaggaccgacagccccaaggcccacgtgacctaccaccccagatcccaggtggacgtgaccctgagatgctgggccctgggcttctaccccgccgatatcaccctgacctggcagctgaacggcgaggatctgacccaggacatggaactcgtggaaacccggcctgccggggacggcacctttcagaaatgggccgctgtggtggtgcccctgggcaaagagcagaactacacctgtcacgtgcaccacaagggcctgcccgagcctctgaccctgcggtggaagctgcctcctagcaccgccagcggcggacatcatcaccatcaccatggacttaatgatatcttcgaggcgcagaagattgaatggcactga

SEQ ID NO: 39 - protein, 466 aa, Artificial Sequence
  CG Protein of Soluble single chain MHC H2Kd molecule with TYQRTRALV peptide with Tags, without the scFv amino acid sequence
MGWSCIILFLVATATGAHSTYQRTRALVGGGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHVSMAEPKTVYWDRDMGGGGSGGGGSGGGGSGGGGSGPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRWKLPPSTASGGHHHHHHGLNDIFEAQKIEWH

SEQ ID NO: 40 - DNA, 2221 bp, Artificial Sequence
  BA DNA of Soluble single chain MHC H2Kd molecule with TYQRTRALV peptide anti MCSP scFv of 225.28S clone and tags nucleic acid sequence
atgggatggagctgtatcatcctcttcttggtagcaacagctacaggtaaggggttaacagtagcaggcttgaggtctggacatatatatgggtgacaatgacatccactttgcctttctctccacaggcgcgcacagtacctaccagcggaccagagccctcgtgggcggcggaggatctggcggaggtggaagtgggggaggcggcagcatccagaaaaccccccagatccaggtgtacagccggcacccccccgagaacggcaagcccaacatcctgaactgctacgtgacccagttccacccccctcacatcgagatccagatgctgaagaatggcaagaagatccccaaggtcgagatgagcgacatgagcttcagcaaggactggtccttctacatcctggcccacaccgagttcacccccaccgaaaccgacacctacgcctgcagagtgaagcacgtgtccatggccgagcccaagaccgtgtactgggaccgggatatgggcggagggggtagcggaggcggtggttctggcggaggtggctcagggggagggggatctggcccccacagcctgcggtacttcgtgaccgccgtgtccagacctggcctgggcgagcctagattcattgccgtgggctacgtggacgacacccagttcgtcagattcgacagcgacgccgacaacccccgcttcgagcccagagccccctggatggaacaggaaggccccgagtactgggaggaacagacccagcgggccaagagcgacgagcagtggttccgggtgtccctgcggaccgcccagcggtactacaaccagagcaagggcggcagccacaccttccagcggatgttcggctgcgacgtgggcagcgactggcggctgctgagaggctaccagcagttcgcctacgacggccgggactatatcgccctgaacgaggacctgaaaacctggacagccgccgacactgccgccctgatcaccagacggaagtgggaacaggccggcgacgctgagtactaccgggcctacctggaaggcgagtgcgtggaatggctgcggagatatctggaactgggcaacgagacactgctgaggaccgacagccccaaggcccacgtgacctaccaccccagatcccaggtggacgtgaccctgagatgctgggccctgggcttctaccccgccgatatcaccctgacctggcagctgaacggcgaggatctgacccaggacatggaactcgtggaaacccggcctgccggggacggcacctttcagaaatgggccgctgtggtggtgcccctgggcaaagagcagaactacacctgtcacgtgcaccacaagggcctgcccgagcctctgaccctgcggtggaagctgcctcctagcaccgccagcggcggacaggtcaaactgcagcagagcggcggaggcctggtgcagcctggcggcagcatgaagctgagctgcgtggtgtccggcttcaccttcagcaactactggatgaactgggtccgacagagccccgagaagggcctggaatggatcgccgagatccggctgaagtccaacaacttcggccggtactacgccgagagcgtgaagggcagattcaccatcagccgggacgacagcaagagcagcgcctacctgcagatgatcaacctgcgggccgaggacaccggcatctactactgcaccagctacggcaactacgtgggccactacttcgaccactggggccagggcaccaccgtgaccgtgtctagcggaggcggaggatctggcggaggtggaagtggcgggggaggcagcgatatcgagctgacccagtcccccaagttcatgagcaccagcgtgggcgaccgggtgtccgtgacatgcaaggccagccagaacgtggacaccaacgtggcctggtatcagcagaagcccggccagagccctgagcccctgctgttcagcgccagctacagatacaccggcgtgcccgacaggttcaccggcagcggctctggcaccgacttcaccctgaccatctccaacgtgcagagcgaggacctggccgagtacttctgccagcagtacaacagctaccccctgacctttggaggcggcaccaagctggaaatcaagcgggcccatcatcaccatcaccatggacttaatgatatcttcgaggcgcagaagattgaatggcactga

SEQ ID NO: 41 - protein, 712 aa, Artificial Sequence
  BA Protein of Soluble single chain MHC H2Kd molecule with TYQRTRALV peptide anti MCSP scFv of 225.28S clone and tags amino acid sequence
MGWSCIILFLVATATGAHSTYQRTRALVGGGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHVSMAEPKTVYWDRDMGGGGSGGGGSGGGGSGGGGSGPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRWKLPPSTASGGQVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSSGGGGSGGGGSGGGGSDIELTQSPKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIKRAHHHHHHGLNDIFEAQKIEWH

SEQ ID NO: 42 - DNA, 2179 bp, Artificial Sequence
  M15 DNA of Soluble single chain MHC H2Kd molecule with TYQRTRALV peptide anti MCSP scFv of 225.28S clone and tags nucleic acid sequence
atgggatggagctgtatcatcctcttcttggtagcaacagctacaggtaaggggttaacagtagcaggcttgaggtctggacatatatatgggtgacaatgacatccactttgcctttctctccacaggcgcgcacagtacctaccagcggaccagagccctcgtgggcggcggaggatctggcggaggtggaagtgggggaggcggcagcatccagaaaaccccccagatccaggtgtacagccggcacccccccgagaacggcaagcccaacatcctgaactgctacgtgacccagttccacccccctcacatcgagatccagatgctgaagaatggcaagaagatccccaaggtcgagatgagcgacatgagcttcagcaaggactggtccttctacatcctggcccacaccgagttcacccccaccgaaaccgacacctacgcctgcagagtgaagcacgtgtccatggccgagcccaagaccgtgtactgggaccgggatatgggcggagggggtagcggaggcggtggttctggcggaggtggctcagggggagggggatctggcccccacagcctgcggtacttcgtgaccgccgtgtccagacctggcctgggcgagcctagattcattgccgtgggctacgtggacgacacccagttcgtcagattcgacagcgacgccgacaacccccgcttcgagcccagagccccctggatggaacaggaaggccccgagtactgggaggaacagacccagcgggccaagagcgacgagcagtggttccgggtgtccctgcggaccgcccagcggtactacaaccagagcaagggcggcagccacaccttccagcggatgttcggctgcgacgtgggcagcgactggcggctgctgagaggctaccagcagttcgcctacgacggccgggactatatcgccctgaacgaggacctgaaaacctggacagccgccgacactgccgccctgatcaccagacggaagtgggaacaggccggcgacgctgagtactaccgggcctacctggaaggcgagtgcgtggaatggctgcggagatatctggaactgggcaacgagacactgctgaggaccgacagccccaaggcccacgtgacctaccaccccagatcccaggtggacgtgaccctgagatgctgggccctgggcttctaccccgccgatatcaccctgacctggcagctgaacggcgaggatctgacccaggacatggaactcgtggaaacccggcctgccggggacggcacctttcagaaatgggccgctgtggtggtgcccctgggcaaagagcagaactacacctgtcacgtgcaccacaagggcctgcccgagcctctgaccctgcggtggaagctgcctcctagcaccgccagcggcggacaggtcaaactgcagcagagcggcggaggcctggtgcagcctggcggcagcatgaagctgagctgcgtggtgtccggcttcaccttcagcaactactggatgaactgggtccgacagagccccgagaagggcctggaatggatcgccgagatccggctgaagtccaacaacttcggccggtactacgccgagagcgtgaagggcagattcaccatcagccgggacgacagcaagagcagcgcctacctgcagatgatcaacctgcgggccgaggacaccggcatctactactgcaccagctacggcaactacgtgggccactacttcgaccactggggccagggcaccaccgtgaccgtgtctagcggaggcggaggatctggcggaggtggaagtggcgggggaggcagcgatatcgagctgacccagtcccccaagttcatgagcaccagcgtgggcgaccgggtgtccgtgacatgcaaggccagccagaacgtggacaccaacgtggcctggtatcagcagaagcccggccagagccctgagcccctgctgttcagcgccagctacagatacaccggcgtgcccgacaggttcaccggcagcggctctggcaccgacttcaccctgaccatctccaacgtgcagagcgaggacctggccgagtacttctgccagcagtacaacagctaccccctgacctttggaggcggcaccaagctggaaatcaagcgggcccatcatcaccatcaccattga

SEQ ID NO: 43 - protein, 698 aa, Artificial Sequence
  M15 Protein of Soluble single chain MHC H2Kd molecule with TYQRTRALV peptide anti MCSP scFv of 225.28S clone and tags amino acid sequence
MGWSCIILFLVATATGAHSTYQRTRALVGGGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHVSMAEPKTVYWDRDMGGGGSGGGGSGGGGSGGGGSGPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRWKLPPSTASGGQVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSSGGGGSGGGGSGGGGSDIELTQSPKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIKRAHHHHHH

SEQ ID NO: 44 - protein, 182 aa, Homo sapiens [sequence omitted]

SEQ ID NO: 45 - protein, 182 aa, Homo sapiens
GSHSMRYFYTSVSRPGRGEPRFIAVGYVDDTQFVRFDSDAASQRMEPRAPWIEQEGPEYWDRNTRNVKAHSQTDRESLRIALRYYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRT

SEQ ID NO: 46 - protein, 182 aa, Homo sapiens
GSHSMRYFTTSVSRPGRGEPRFIAVGYVDDTQFVRFDSDAASQRMEPRAPWIEQEGPEYWDLQTRNVKAQSQTDRANLGTLRGYYNQSEAGSHTIQMMYGCHVGSDGRFLRGYRQDAYDGKDYIALNEDLRSWTAADMAAQITQRKWEAARVAEQLRAYLEGTCVEWLRRYLENGKETLQRT

SEQ ID NO: 47 - protein, 182 aa, Homo sapiens [sequence omitted]

SEQ ID NO: 48 - protein, 182 aa, Homo sapiens
GSHSMRYFYTSVSRPGRGEPRFIAVGYVDDTQFVRFDSDAASQRMEPRAPWIEQEGPEYWDRNTRKVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGTCVEWLRRYLENGKETLQRT

SEQ ID NO: 49 - protein, 182 aa, Homo sapiens
GSHSMRYFFTSVSRPGRGEPRFIAVGYVDDSQFVQFDSDAASQRMEPRAPWIEQEEPEYWDEETRNVKAHSQTNRANLGTLRGYYNQSEDGSHTIQIMYGCDVGSDGRFLRGYRQDAYDGKDYIALNEDLRSWTAADMAAQITKRKWEAARRAEQLRAYLEGECVDGLRRYLENGKETLQRT

SEQ ID NO: 50 - protein, 182 aa, Homo sapiens
GSHSMRYFYTSVSRPGRGEPRFIAVGYVDDTQFVRFDSDAASQRMEPRAPWIEQEGPEYWDRNTRNVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRT

SEQ ID NO: 51 - protein, 182 aa, Homo sapiens
GSHSMRYFYTSVSRPGRGEPRFIAVGYVDDTQFVRFDSDAASQRMEPRAPWIEQEGPEYWDRNTRNVKAQSQTDRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRT

SEQ ID NO: 52 - protein, 182 aa, Homo sapiens [sequence omitted]

SEQ ID NO: 53 - protein, 183 aa, Homo sapiens
GSHSMRYFYTSVSRPGRGEPRFIAVGYVDDTQFVRFDSDAASQRMEPRAPWIEQEGPEYWDLQTRNVKAHSQTDRANLGTLRGYYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRTD

SEQ ID NO: 54 - protein, 182 aa, Homo sapiens [sequence omitted]

SEQ ID NO: 55 - protein, 182 aa, Homo sapiens
GSHSMRYFFTSVSRPGRGEPRFIAVGYVDDTQFVRFDSDAASQRMEPRAPWIEQEGPEYWDQETRNVKAHSQTDRVDLGTLRGYYNQSEAGSHTIQMMYGCDVGPDGRLLRGYQQDAYDGKDYIALNEDLRSWTAADMAAQITQRKWEAARVAEQLRAYLEGTCVEWLRRYLENGKETLQRT

SEQ ID NO: 56 - protein, 182 aa, Homo sapiens
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGTCVEWLRRYLENGKETLQRT

SEQ ID NO: 57 - protein, 182 aa, Homo sapiens [sequence omitted]

SEQ ID NO: 58 - protein, 182 aa, Homo sapiens
GSHSMRYFYTSVSRPGRGEPRFISVGYVDDTQFVRFDSDAASPREEPRAPWIEQEGPEYWDRNTQICKTNTQTDRESLRNLRGYYNQSEAGSHTLQWMYGCDVGPDGRLLRGYNQFAYDGKDYIALNEDLSSWTAADTAAQITQRKWEAAREAEQLRAYLEGTCVEWLRRHLENGKETLQRA

SEQ ID NO: 59 - protein, 182 aa, Homo sapiens [sequence omitted]

SEQ ID NO: 60 - protein, 182 aa, Homo sapiens
GSHSMRYFHTSVSRPGRGEPRFITVGYVDDTLFVRFDSDAASPREEPRAPWIEQEGPEYWDRETQICKAKAQTDRENLRIALRYYNQSEAGSHTLQNMYGCDVGPDGRLLRGYHQDAYDGKDYIALNEDLSSWTAADTAAQITQRKWEAARVAEQLRAYLEGECVEWLRRYLENGKETLQRA

SEQ ID NO: 61 - protein, 182 aa, Homo sapiens
GSHSMRYFYTSVSRPGRGEPRFISVGYVDDTQFVRFDSDAASPREEPRAPWIEQEGPEYWDRNTQICKTNTQTYRENLRIALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRA

SEQ ID NO: 62 - protein, 182 aa, Homo sapiens
GSHSMRYFYTSVSRPGRGEPRFISVGYVDDTQFVRFDSDAASPREEPRAPWIEQEGPEYWDRNTQICKTNTQTDRESLRNLRGYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRA

SEQ ID NO: 63 - protein, 182 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTWQRMYGCDVGPDGRLLRGHNQYAYDGKDYIALNEDLRSWTAADTAAQITQRKWEAARVAEQDRAYLEGTCVEWLRRYLENGKDTLERA

SEQ ID NO: 64 - protein, 182 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERA

SEQ ID NO: 65 - protein, 181 aa, Homo sapiens
GSHSMRYFYTAMSRPGRGEPRFITVGYVDDTLFVRFDSDATSPRKEPRAPWIEQEGPEYWDRETQISKTNTQTYREDLRTLLRYYNQSEAGSHTLQRMFGCDVGPDGRLLRGYHQDAYDGKDYIALNEDLSSWTAADTAAQITQRKWEAARVAEQLRAYLEGECVEWLRRYLENGKETLQR

SEQ ID NO: 66 - protein, 182 aa, Homo sapiens
GSHSMRYFYTSVSRPGRGEPRFISVGYVDDTQFVRFDSDAASPREEPRAPWIEQEGPEYWDRETQISKTNTQTYRESLRNLRGYYNQSEAGSHTLQSMYGCDVGPDGRLLRGHNQYAYDGKDYIALNEDLRSWTAADTAAQISQRKLEAARVAEQLRAYLEGECVEWLRRYLENGKDKLERA

SEQ ID NO: 67 - protein, 182 aa, Homo sapiens
GSHSMRYFHTAMSRPGRGEPRFITVGYVDDTLFVRFDSDATSPRKEPRAPWIEQEGPEYWDRETQISKTNTQTYRENLRIALRYYNQSEAGSHTWQRMYGCDLGPDGRLLRGYNQLAYDGKDYIALNEDLSSWTAADTAAQITQRKWEAAREAEQLRAYLEGLCVEWLRRYLENGKETLQRA

SEQ ID NO: 68 - protein, 182 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTWQRMYGCDLGPDGRLLRGYNQLAYDGKDYIALNEDLSSWTAADTAAQITQRKWEAAREAEQLRAYLEGLCVEWLRRYLENGKETLQRA

SEQ ID NO: 69 - protein, 182 aa, Homo sapiens
GSHSMRYFYTAMSRPGRGEPRFIAVGYVDDTQFVRFDSDAASPRTEPRAPWIEQEGPEYWDRETQISKTNTQTYRENLRIALRYYNQSEAGSHTWQTMYGCDVGPDGRLLRGHNQYAYDGKDYIALNEDLSSWTAADTAAQITQRKWEAAREAEQLRAYLEGLCVEWLRRHLENGKETLQRA

SEQ ID NO: 70 - protein, 182 aa, Homo sapiens [sequence omitted]

SEQ ID NO: 71 - protein, 182 aa, Homo sapiens [sequence omitted]

SEQ ID NO: 72 - protein, 182 aa, Homo sapiens
GSHSMRYFYTAMSRPGRGEPRFIAVGYVDDTQFVRFDSDAASPREEPRAPWIEQEGPEYWDRNTQIYKAQAQTDRESLRNLRGYYNQSEAGSHTWQTMYGCDLGPDGRLLRGHNQLAYDGKDYIALNEDLSSWTAADTAAQITQRKWEAAREAEQLRAYLEGTCVEWLRRYLENGKETLQRA

SEQ ID NO: 73 - protein, 182 aa, Homo sapiens
GSHSMRYFYTAMSRPGRGEPRFIAVGYVDDTQFVRFDSDAASPREEPRAPWIEQEGPEYWDRNTQIYKAQAQTDRESLRNLRGYYNQSEAGSHTWQTMYGCDLGPDGRLLRGHNQLAYDGKDYIALNEDLSSWTAADTAAQITQRKWEAARVAEQLRAYLEGLCVEWLRRYLENGKETLQRA

SEQ ID NO: 74 - protein, 182 aa, Homo sapiens [sequence omitted]

SEQ ID NO: 75 - protein, 182 aa, Homo sapiens [sequence omitted]

SEQ ID NO: 76 - protein, 182 aa, Homo sapiens
GSHSMRYFYTAMSRPGRGEPRFIAVGYVDDTQFVRFDSDAASPREEPRAPWIEQEGPEYWDRNTQIFKTNTQTYRENLRIALRYYNQSEAGSHTWQTMYGCDLGPDGRLLRGHNQLAYDGKDYIALNEDLSSWTAADTAAQITQRKWEAARVAEQLRAYLEGTCVEWLRRYLENGKETLQRA

SEQ ID NO: 77 - protein, 182 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRA

SEQ ID NO: 78 - protein, 182 aa, Homo sapiens
GSHSMRYFHTSVSRPGRGEPRFITVGYVDDTQFVRFDSDAASPREEPRAPWIEQEGPEYWDRNTQICKAKAQTDRVGLRNLRGYYNQSEDGSHTWQTMYGCDMGPDGRLLRGYNQFAYDGKDYIALNEDLRSWTAADTAAQITQRKWEAARVAEQLRAYLEGECVEWLRRHLENGKETLQRA

SEQ ID NO: 79 - protein, 182 aa, Homo sapiens
GSHSMRYFYTAMSRPGRGEPRFIAVGYVDDTQFVRFDSDAASPRTEPRAPWIEQEGPEYWDRNTQIFKTNTQTDRESLRNLRGYYNQSEAGSHTWQTMYGCDVGPDGRLLRGHNQYAYDGKDYIALNEDLSSWTAADTAAQITQRKWEAAREAEQLRAYLEGLCVEWLRRHLENGKETLQRA

SEQ ID NO: 80 - protein, 182 aa, Homo sapiens
GSHSMRYFYTAMSRPGRGEPRFISVGYVDDTQFVRFDSDAASPREEPRAPWIEQEGPEYWDRNTQIYKAQAQTDRESLRNLRGYYNQSEAGSHTLQRMFGCDLGPDGRLLRGHNQLAYDGKDYIALNEDLSSWTAADTAAQITQRKWEAARVAEQDRAYLEDLCVESLRRYLENGKETLQRA

SEQ ID NO: 81 - protein, 182 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHNQYAYDGKDYIALNEDLRSWTAADTAAQISQRKLEAARVAEQLRAYLEGECVEWLRRYLENGKDKLERA

SEQ ID NO: 82 - DNA, 357 bp, Homo sapiens
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatg

SEQ ID NO: 83 - protein, 99 aa, Homo sapiens [sequence omitted]

SEQ ID NO: 84 - protein, 9 aa, Ebola virus
AYQGDYKLF

SEQ ID NO: 85 - protein, 10 aa, Dengue virus
RFLEFEALGF

SEQ ID NO: 86 - protein, 9 aa, Vaccinia virus
VWINNSWKF

SEQ ID NO: 87 - protein, 9 aa, Vaccinia virus
TYNDHIVNL

SEQ ID NO: 88 - protein, 10 aa, Human herpesvirus 5
AYAQKIFKIL

SEQ ID NO: 89 - protein, 9 aa, Human herpesvirus 4
PYLFWLAAI

SEQ ID NO: 90 - protein, 9 aa, Yellow fever virus
IYGIFQSTF

SEQ ID NO: 91 - protein, 9 aa, Homo sapiens
LWMRLLPLL

SEQ ID NO: 92 - protein, 9 aa, Influenza A virus
FYIQMCTEL

SEQ ID NO: 93 - protein, 9 aa, Plasmodium falciparum
SFLFVEALF

SEQ ID NO: 94 - protein, 9 aa, Plasmodium falciparum
VFNVVNSSI

SEQ ID NO: 95 - protein, 9 aa, Homo sapiens
YFDPANGKF

SEQ ID NO: 96 - protein, 9 aa, Influenza A virus
YYLEKANKI

SEQ ID NO: 97 - protein, 10 aa, Influenza A virus
FYRYGFVANF

SEQ ID NO: 98 - protein, 9 aa, Homo sapiens
KYFDEHYEY

SEQ ID NO: 99 - protein, 9 aa, Dengue virus
IQKETLVTF

SEQ ID NO: 100 - protein, 10 aa, Dengue virus
IQMSSGNLLF

SEQ ID NO: 101 - protein, 9 aa, Dengue virus
SYSMCTGKF

SEQ ID NO: 102 - protein, 9 aa, Homo sapiens
AYVPGFAHI

SEQ ID NO: 103 - protein, 9 aa, Homo sapiens
KYLSVQGQF

SEQ ID NO: 104 - protein, 9 aa, Homo sapiens
KYQEVTNNL

SEQ ID NO: 105 - protein, 9 aa, Homo sapiens
LYDPVISKL

SEQ ID NO: 106 - protein, 9 aa, Homo sapiens
RYIANTVEL

SEQ ID NO: 107 - protein, 9 aa, Homo sapiens
RYLEQLHQL

SEQ ID NO: 108 - protein, 442 aa, Artificial Sequence
  Example of hCG Protein of Soluble single chain HLA-A*230101 molecule with PYLFWLAAI peptide with Tags, without the scFv amino acid sequence
PYLFWLAAIGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEASGGHHHHHHGLNDIFEAQKIEWH

SEQ ID NO: 109 - protein, 682 aa, Artificial Sequence
  Example of hBAScFv Protein of Soluble single chain HLA-A*230101 molecule with PYLFWLAAI peptide and an anti MSLN ScFv with Tags amino acid sequence
PYLFWLAAIGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEASGGQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGAGTKLEIKHHHHHHGLNDIFEAQKIEWH

SEQ ID NO: 110 - protein, 675 aa, Artificial Sequence
  Example of hM15ScFv Protein of Soluble single chain HLA-A*230101 molecule with PYLFWLAAI peptide and an anti MSLN Fab with Tags amino acis sequence
PYLFWLAAIPEPTIDEGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEASGGQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGAGTKLEIKHHHHHH

SEQ ID NO: 111 - protein, 665 aa, Artificial Sequence
  Example of hBAFab(human Kappa) Protein of Soluble single chain HLA-A*230101 molecule with PYLFWLAAI peptide and an anti MSLN ScFv with Tags. T cell Engaging domain and Fab Heavy Chain
PYLFWLAAIGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEASGGQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCVHHHHHHGLNDIFEAQKIEWH

SEQ ID NO: 112 - protein, 212 aa, Artificial Sequence
  Example of hBAFab(human Kappa) Protein of Soluble single chain HLA-A*230101 molecule with PYLFWLAAI peptide and an anti MSLN ScFv with Tags. T cell Engaging domain and Fab light Chain
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGAGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 184 aa, Homo sapiens
MAPLLPIRTLPLILILLALLSPGAADFNISSLSGLLSPALTESLLVALPPCHLTGGNATLMVRRANDSKVVTSSFVVPPCRGRRELVSVVDSGAGFTVTRLSAYQVTNLVPGTKFYISYLVKKGTATESSREIPMSTLPRRNMESIGLGMARTGGMVVITVLLSVAMFLLVLGFIIALALGSRK

SEQ ID NO: 114 - protein, 273 aa, Homo sapiens
MASAAAAEAEKGSPVVVGLLVVGNIIILLSGLSLFAETIWVTADQYRVYPLMGVSGKDDVFAGAWIAIFCGFSFFMVASFGVGAALCRRRSMVLTYLVLMLIVYIFECASCITSYTHRDYMVSNPSLITKQMLTFYSADTDQGQELTRLWDRVMIEQECCGTSGPMDWVNFTSAFRAATPEVVFPWPPLCCRRTGNFIPLNEEGCRLGHMDYLFTKAGVQWHNLSSLQRLPPGFKGFSHLSFQSSWDYRAASNTSATPSTATRGVSRGLGLPS

SEQ ID NO: 115 - protein, 258 aa, Homo sapiens
MASAAAAEAEKGSPVVVGLLVVGNIIILLSGLSLFAETIWVTADQYRVYPLMGVSGKDDVFAGAWIAIFCGFSFFMVASFGVGAALCRRRSMVLTYLVLMLIVYIFECASCITSYTHRDYMVSNPSLITKQMLTFYSADTDQGQELTRLWDRVMIEQECCGTSGPMDWVNFTSAFRAATPEVVFPWPPLCCRRTGNFIPLNEEGCRLGHMDYLFTKGCFEHIGHAIDSYTWGISWFGFAILMWTLPVMLIAMYFYTML

SEQ ID NO: 116 - protein, 245 aa, Homo sapiens [sequence omitted]

SEQ ID NO: 117 - protein, 114 aa, Homo sapiens
MAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 118 - protein, 386 aa, Homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 119 - protein, 418 aa, Homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQVLKVIFAVAFCLISAVLMVLLFIHIRRGLCWQRESYGNI

SEQ ID NO: 120 - protein, 353 aa, Homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 121 - protein, 335 aa, Homo sapiens
MPRPRLLAALCGALLCAPSLLVALDICSKNPCHNGGLCEEISQEVRGDVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQIFPGNWDNHSHKKNLFETPILARYVRILPVAWHNRIALRLELLGC

SEQ ID NO: 122 - protein, 387 aa, Homo sapiens
MPRPRLLAALCGALLCAPSLLVALDICSKNPCHNGGLCEEISQEVRGDVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 123 - protein, 264 aa, Homo sapiens
MTPGTQSPFFLLLLLTVLTATTAPKPATVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 124 - protein, 255 aa, Homo sapiens
MTPGTQSPFFLLLLLTVLTVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 125 - protein, 203 aa, Homo sapiens
MTPGTQSPFFLLLLLTVLTVVTGSGHASSTPGGEKETSATQRSSVPSSTEKNAIPAPTTTKSCRETFLKCFCRFINKGVFWASPILSSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 126 - protein, 150 aa, Homo sapiens
MTPGTQSPFFLLLLLTVLTVVTGSGHASSTPGGEKETSATQRSSVPSSTEKNAFNSSLEDPSTDYYQELQRDISEMAVCQCRRKNYGQLDIFPARDTYHPMSEYPTYHTHGRYVPPSSTDRSPYEKVSAGNGGSSLSYTNPAVAATSANL

SEQ ID NO: 127 - protein, 159 aa, Homo sapiens
MTPGTQSPFFLLLLLTVLTATTAPKPATVVTGSGHASSTPGGEKETSATQRSSVPSSTEKNAFNSSLEDPSTDYYQELQRDISEMAVCQCRRKNYGQLDIFPARDTYHPMSEYPTYHTHGRYVPPSSTDRSPYEKVSAGNGGSSLSYTNPAVAATSANL

SEQ ID NO: 128 - protein, 158 aa, Homo sapiens
MTPGTQSPFFLLLLLTVLTVVTGSGHASSTPGGEKETSATQRSSVPSSTEKNAIPAPTTTKSCRETFLKCFCRFINKGVFWASPILSSVWGWGARLGHRAAGAGLCSGCAGHCLSHCLGCLSVPPKELRAAGHLSSPGYLPSYERVPHLPHPWALCAP

SEQ ID NO: 129 - protein, 475 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 130 - protein, 484 aa, Homo sapiens
MTPGTQSPFFLLLLLTVLTATTAPKPATVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 131 - protein, 282 aa, Homo sapiens
MTPGTQSPFFLLLLLTVLTATTAPKPATVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 132 - protein, 219 aa, Homo sapiens
MTPGTQSPFFLLLLLTVLTATTAPKPATVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVWGWGARLGHRAAGAGLCSGCAGHCLSHCLGCLSVPPKELRAAGHLSSPGYLPSYERVPHLPHPWALCAP

SEQ ID NO: 133 - protein, 238 aa, Homo sapiens
MTPGTQSPFFLLLLLTVLTATTAPKPATVVTGSGHASSTPGGEKETSATQRSSVPSSTEKNAIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 134 - protein, 217 aa, Homo sapiens
MTPGTQSPFFLLLLLTVLTGGEKETSATQRSSVPSSTEKNAIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 135 - protein, 241 aa, Homo sapiens
MTPGTQSPFFLLLLLTVLTATTAPKPATVVTGSGHASSTPGGEKETSATQRSSVPSSTEKNAFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 136 - protein, 239 aa, Homo sapiens
MTPGTQSPFFLLLLLTVLTATTAPKPATVVTGSGHASSTPGGEKETSATQRSSVPSSTEKNAIPAPTTTKSCRETFLKWPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 137 - protein, 198 aa, Homo sapiens
MTPGTQSPFFLLLLLTVLTVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSGCLSVPPKELRAAGHLSSPGYLPSYERVPHLPHPWALCAP

SEQ ID NO: 138 - protein, 230 aa, Homo sapiens
MTPGTQSPFFLLLLLTVLTVVTGSGHASSTPGGEKETSATQRSSVPSSTEKNAIPAPTTTKSCRETFLKWPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 139 - protein, 189 aa, Homo sapiens
MTPGTQSPFFLLLLLTVLTATTAPKPATVVTGSGHASSTPGGEKETSATQRSSVPSSTEKNAFNSSLEDPSTDYYQELQRDISEMSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 140 - protein, 212 aa, Homo sapiens
MTPGTQSPFFLLLLLTVLTATTAPKPATVVTGSGHASSTPGGEKETSATQRSSVPSSTEKNAIPAPTTTKSCRETFLKCFCRFINKGVFWASPILSSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 141 - protein, 177 aa, Homo sapiens
MTPGTQSPFFLLLLLTVLTATTAPKPATVVTGSGHASSTPGGEKETSATQRSSVPSSTEKNALSTGVSFFFLSFHISNLQFNSSLEDPSTDYYQELQRDISEMAVCQCRRKNYGQLDIFPARDTYHPMSEYPTYHTHGRYVPPSSTDRSPYEKVSAGNGGSSLSYTNPAVAATSANL

SEQ ID NO: 142 - protein, 273 aa, Homo sapiens
MTPGTQSPFFLLLLLTVLTVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 143 - protein, 575 aa, Homo sapiens
MDLVLKRCLLHLAVIGALLAVGATKGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 144 - protein, 668 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPVPGILLTGQEAGLGQVPLIVGILLVLMAVVLASLIYRRRLMKQDFSVPQLPHSSSHWLRLPRIFCSCPIGENSPLLSGQQV

SEQ ID NO: 145 - protein, 661 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 146 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 147 - protein, 1069 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 148 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 149 - protein, 353 aa, Human T-lymphotropic virus 1
MAHFPGFGQSLLFGYPVYVFGDCVQGDWCPISGGLCSARLHRHALLATCPEHQITWDPIDGRVIGSALQFLIPRLPSFPTQRTSKTLKVLTPPITHTTPNIPPSFLQAMRKYSPFRNGYMEPTLGQHLPTLSFPDPGLRPQNLYTLWGGSVVCMYLYQLSPPITWPLLPHVIFCHPGQLGAFLTNVPYKRIEKLLYKISLTTGALIILPEDCLPTTLFQPARAPVTLTAWQNGLLPFHSTLTTPGLIWTFTDGTPMISGPCPKDGQPSLVLQSSSFIFHKFQTKAYHPSFLLSHGLIQYSSFHNLHLLFEEYTNIPISLLFNEKEADDNDHEPQISPGGLEPLSEKHFRETEV

SEQ ID NO: 150 - protein, 345 aa, Human T-lymphotropic virus 4
MAHFPGFGQSLLYGYPVYVFGDCVQADWCPISGGLCSPRLHRHALLATCPEHQITWDPIDGRVVGSPLQYLIPRLPSFPTQRTSKTLKVLTPPTTPVTPKVPPSFFQSVRRHSPYRNGCLETTLGEQLPSLAFPEPGLRPQNVYTIWGKTIVCLYIYQLSPPMTWPLIPHVIFCNPRQLGAFLSNVPPKRLEELLYKLYLHTGAIIILPEDALPTTLFQPVRAPCVQTTWNTGLLPYQPNLTTPGLIWTFNDGSPMISGPCPKAGQPSLVVQSSLLIFERFQTKAYHPSYLLSHQLIQYSSFHHLYLLFDEYTTIPFSLLFKEKEGDDRDNDPLPGATASPQGQN

SEQ ID NO: 151 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 152 - protein, 309 aa, Homo sapiens
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 153 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 154 - protein, 1225 aa, Homo sapiens
MKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 155 - protein, 1240 aa, Homo sapiens
MPRGSWKPQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 156 - protein, 1055 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRNM

SEQ ID NO: 157 - protein, 603 aa, Homo sapiens
MKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHS

SEQ ID NO: 158 - protein, 1255 aa, Homo sapiens [sequence omitted]

SEQ ID NO: 159 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 160 - protein, 781 aa, Homo sapiens; identical to SEQ ID NO: 159

SEQ ID NO: 161 - protein, 781 aa, Homo sapiens; identical to SEQ ID NO: 159

SEQ ID NO: 162 - protein, 529 aa, Homo sapiens [sequence omitted]

SEQ ID NO: 163 - protein, 2322 aa, Homo sapiens
MQSGRGPPLPAPGLALALTLTMLARLASAASFFGENHLEVPVATALTDIDLQLQFSTSQPEALLLLAAGPADHLLLQLYSGRLQVRLVLGQEELRLQTPAETLLSDSIPHTVVLTVVEGWATLSVDGFLNASSAVPGAPLEVPYGLFVGGTGTLGLPYLRGTSRPLRGCLHAATLNGRSLLRPLTPDVHEGCAEEFSASDDVALGFSGPHSLAAFPAWGTQDEGTLEFTLTTQSRQAPLAFQAGGRRGDFIYVDIFEGHLRAVVEKGQGTVLLHNSVPVADGQPHEVSVHINAHRLEISVDQYPTHTSNRGVLSYLEPRGSLLLGGLDAEASRHLQEHRLGLTPEATNASLLGCMEDLSVNGQRRGLREALLTRNMAAGCRLEEEEYEDDAYGHYEAFSTLAPEAWPAMELPEPCVPEPGLPPVFANFTQLLTISPLVVAEGGTAWLEWRHVQPTLDLMEAELRKSQVLFSVTRGAHYGELELDILGAQARKMFTLLDVVNRKARFIHDGSEDTSDQLVLEVSVTARVPMPSCLRRGQTYLLPIQVNPVNDPPHIIFPHGSLMVILEHTQKPLGPEVFQAYDPDSACEGLTFQVLGTSSGLPVERRDQPGEPATEFSCRELEAGSLVYVHCGGPAQDLTFRVSDGLQASPPATLKVVAIRPAIQIHRSTGLRLAQGSAMPILPANLSVETNAVGQDVSVLFRVTGALQFGELQKHSTGGVEGAEWWATQAFHQRDVEQGRVRYLSTDPQHHAYDTVENLALEVQVGQEILSNLSFPVTIQRATVWMLRLEPLHTQNTQQETLTTAHLEATLEEAGPSPPTFHYEVVQAPRKGNLQLQGTRLSDGQGFTQDDIQAGRVTYGATARASEAVEDTFRFRVTAPPYFSPLYTFPIHIGGDPDAPVLTNVLLVVPEGGEGVLSADHLFVKSLNSASYLYEVMERPRLGRLAWRGTQDKTTMVTSFTNEDLLRGRLVYQHDDSETTEDDIPFVATRQGESSGDMAWEEVRGVFRVAIQPVNDHAPVQTISRIFHVARGGRRLLTTDDVAFSDADSGFADAQLVLTRKDLLFGSIVAVDEPTRPIYRFTQEDLRKRRVLFVHSGADRGWIQLQVSDGQHQATALLEVQASEPYLRVANGSSLVVPQGGQGTIDTAVLHLDTNLDIRSGDEVHYHVTAGPRWGQLVRAGQPATAFSQQDLLDGAVLYSHNGSLSPEDTMAFSVEAGPVHTDATLQVTIALEGPLAPLKLVRHKKIYVFQGEAAEIRRDQLEAAQEAVPPADIVFSVKSPPSAGYLVMVSRGALADEPPSLDPVQSFSQEAVDTGRVLYLHSRPEAWSDAFSLDVASGLGAPLEGVLVELEVLPAAIPLEAQNFSVPEGGSLTLAPPLLRVSGPYFPTLLGLSLQVLEPPQHGPLQKEDGPQARTLSAFSWRMVEEQLIRYVHDGSETLTDSFVLMANASEMDRQSHPVAFTVTVLPVNDQPPILTTNTGLQMWEGATAPIPAEALRSTDGDSGSEDLVYTIEQPSNGRVVLRGAPGTEVRSFTQAQLDGGLVLFSHRGTLDGGFPFRLSDGEHTSPGHFFRVTAQKQVLLSLKGSQTLTVCPGSVQPLSSQTLRASSSAGTDPQLLLYRVVRGPQLGRLFHAQQDSTGEALVNFTQAEVYAGNILYEHEMPPEPFWEAHDTLELQLSSPPARDVAATLAVAVSFEAACPQRPSHLWKNKGLWVPEGQRARITVAALDASNLLASVPSPQRSEHDVLFQVTQFPSRGQLLVSEEPLHAGQPHFLQSQLAAGQLVYAHGGGGTQQDGFHFRAHLQGPAGASVAGPQTSEAFAITVRDVNERPPQPQASVPLRLTRGSRAPISRAQLSVVDPDSAPGEIEYEVQRAPHNGFLSLVGGGLGPVTRFTQADVDSGRLAFVANGSSVAGIFQLSMSDGASPPLPMSLAVDILPSAIEVQLRAPLEVPQALGRSSLSQQQLRVVSDREEPEAAYRLIQGPQYGHLLVGGRPTSAFSQFQIDQGEVVFAFTNFSSSHDHFRVLALARGVNASAVVNVTVRALLHVWAGGPWPQGATLRLDPTVLDAGELANRTGSVPRFRLLEGPRHGRVVRVPRARTEPGGSQLVEQFTQQDLEDGRLGLEVGRPEGRAPGPAGDSLTLELWAQGVPPAVASLDFATEPYNAARPYSVALLSVPEAARTEAGKPESSTPTGEPGPMASSPEPAVAKGGFLSFLEANMFSVIIPMCLVLLLLALILPLLFYLRKRNKTGKHDVQVLTAKPRNGLAGDTETFRKVEPGQAIPLTAVPGQGPPPGGQPDPELLQFCRTPNPALKNGQYWV

SEQ ID NO: 164 - protein, 693 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEIT

SEQ ID NO: 165 - protein, 464 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 166 - protein, 538 aa, Homo sapiens
MEGGRRARVVIESKRNFFLGAFPTPFPAEHVELGRLGDSETAMVPGKGGADYILLPFKKMDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 167 - protein, 496 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRFHFCRMSWAEANSQCQTQSVPFWRRVDHLLIRVMLYQISEEVSRSELRSFKFLLQEEISKCKLDDDMNLLDIFIEMEKRVILGEGKLDILKRVCAQINKSLLKIINDYEEFSKGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 168 - protein, 479 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 169 - protein, 464 aa, Homo sapiens; identical to SEQ ID NO: 165

SEQ ID NO: 170 - protein, 235 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNDFGQSLPNEKQTSGILSDHQQSQFCKSTGESAQTSQH

SEQ ID NO: 171 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 172 - protein, 3312 aa, Homo sapiens
MMARRPPWRGLGGRSTPILLLLLLSLFPLSQEELGGGGHQGWDPGLAATTGPRAHIGGGALALCPESSGVREDGGPGLGVREPIFVGLRGRRQSARNSRGPPEQPNEELGIEHGVQPLGSRERETGQGPGSVLYWRPEVSSCGRTGPLQRGSLSPGALSSGVPGSGNSSPLPSDFLIRHHGPKPVSSQRNAGTGSRKRVGTARCCGELWATGSKGQGERATTSGAERTAPRRNCLPGASGSGPELDSAPRTARTAPASGSAPRESRTAPEPAPKRMRSRGLFRCRFLPQRPGPRPPGLPARPEARKVTSANRARFRRAANRHPQFPQYNYQTLVPENEAAGTAVLRVVAQDPDAGEAGRLVYSLAALMNSRSLELFSIDPQSGLIRTAAALDRESMERHYLRVTAQDHGSPRLSATTMVAVTVADRNDHSPVFEQAQYRETLRENVEEGYPILQLRATDGDAPPNANLRYRFVGPPAARAAAAAAFEIDPRSGLISTSGRVDREHMESYELVVEASDQGQEPGPRSATVRVHITVLDENDNAPQFSEKRYVAQVREDVRPHTVVLRVTATDRDKDANGLVHYNIISGNSRGHFAIDSLTGEIQVVAPLDFEAEREYALRIRAQDAGRPPLSNNTGLASIQVVDINDHIPIFVSTPFQVSVLENAPLGHSVIHIQAVDADHGENARLEYSLTGVAPDTPFVINSATGWVSVSGPLDRESVEHYFFGVEARDHGSPPLSASASVTVTVLDVNDNRPEFTMKEYHLRLNEDAAVGTSVVSVTAVDRDANSAISYQITGGNTRNRFAISTQGGVGLVTLALPLDYKQERYFKLVLTASDRALHDHCYVHINITDANTHRPVFQSAHYSVSVNEDRPMGSTIVVISASDDDVGENARITYLLEDNLPQFRIDADSGAITLQAPLDYEDQVTYTLAITARDNGIPQKADTTYVEVMVNDVNDNAPQFVASHYTGLVSEDAPPFTSVLQISATDRDAHANGRVQYTFQNGEDGDGDFTIEPTSGIVRTVRRLDREAVSVYELTAYAVDRGVPPLRTPVSIQVMVQDVNDNAPVFPAEEFEVRVKENSIVGSVVAQITAVDPDEGPNAHIMYQIVEGNIPELFQMDIFSGELTALIDLDYEARQEYVIVVQATSAPLVSRATVHVRLVDQNDNSPVLNNFQILFNNYVSNRSDTFPSGIIGRIPAYDPDVSDHLFYSFERGNELQLLVVNQTSGELRLSRKLDNNRPLVASMLVTVTDGLHSVTAQCVLRVVIITEELLANSLTVRLENMWQERFLSPLLGRFLEGVAAVLATPAEDVFIFNIQNDTDVGGTVLNVSFSALAPRGAGAGAAGPWFSSEELQEQLYVRRAALAARSLLDVLPFDDNVCLREPCENYMKCVSVLRFDSSAPFLASASTLFRPIQPIAGLRCRCPPGFTGDFCETELDLCYSNPCRNGGACARREGGYTCVCRPRFTGEDCELDTEAGRCVPGVCRNGGTCTDAPNGGFRCQCPAGGAFEGPRCEVAARSFPPSSFVMFRGLRQRFHLTLSLSFATVQQSGLLFYNGRLNEKHDFLALELVAGQVRLTYSTGESNTVVSPTVPGGLSDGQWHTVHLRYYNKPRTDALGGAQGPSKDKVAVLSVDDCDVAVALQFGAEIGNYSCAAAGVQTSSKKSLDLTGPLLLGGVPNLPENFPVSHKDFIGCMRDLHIDGRRVDMAAFVANNGTMAGCQAKLHFCDSGPCKNSGFCSERWGSFSCDCPVGFGGKDCQLTMAHPHHFRGNGTLSWNFGSDMAVSVPWYLGLAFRTRATQGVLMQVQAGPHSTLLCQLDRGLLSVTVTRGSGRASHLLLDQVTVSDGRWHDLRLELQEEPGGRRGHHVLMVSLDFSLFQDTMAVGSELQGLKVKQLHVGGLPPGSAEEAPQGLVGCIQGVWLGSTPSGSPALLPPSHRVNAEPGCVVTNACASGPCPPHADCRDLWQTFSCTCQPGYYGPGCVDACLLNPCQNQGSCRHLPGAPHGYTCDCVGGYFGHHCEHRMDQQCPRGWWGSPTCGPCNCDVHKGFDPNCNKTNGQCHCKEFHYRPRGSDSCLPCDCYPVGSTSRSCAPHSGQCPCRPGALGRQCNSCDSPFAEVTASGCRVLYDACPKSLRSGVWWPQTKFGVLATVPCPRGALGAAVRLCDEAQGWLEPDLFNCTSPAFRELSLLLDGLELNKTALDTMEAKKLAQRLREVTGHTDHYFSQDVRVTARLLAHLLAFESHQQGFGLTATQDAHFNENLLWAGSALLAPETGDLWAALGQRAPGGSPGSAGLVRHLEEYAATLARNMELTYLNPMGLVTPNIMLSIDRMEHPSSPRGARRYPRYHSNLFRGQDAWDPHTHVLLPSQSPRPSPSEVLPTSSSIENSTTSSVVPPPAPPEPEPGISIIILLVYRTLGGLLPAQFQAERRGARLPQNPVMNSPVVSVAVFHGRNFLRGILESPISLEFRLLQTANRSKAICVQWDPPGLAEQHGVWTARDCELVHRNGSHARCRCSRTGTFGVLMDASPRERLEGDLELLAVFTHVVVAVSVAALVLTAAILLSLRSLKSNVRGIHANVAAALGVAELLFLLGIHRTHNQLVCTAVAILLHYFFLSTFAWLFVQGLHLYRMQVEPRNVDRGAMRFYHALGWGVPAVLLGLAVGLDPEGYGNPDFCWISVHEPLIWSFAGPVVLVIVMNGTMFLLAARTSCSTGQREAKKTSALTLRSSFLLLLLVSASWLFGLLAVNHSILAFHYLHAGLCGLQGLAVLLLFCVLNADARAAWMPACLGRKAAPEEARPAPGLGPGAYNNTALFEESGLIRITLGASTVSSVSSARSGRTQDQDSQRGRSYLRDNVLVRHGSAADHTDHSLQAHAGPTDLDVAMFHRDAGADSDSDSDLSLEEERSLSIPSSESEDNGRTRGRFQRPLCRAAQSERLLTHPKDVDGNDLLSYWPALGECEAAPCALQTWGSERRLGLDTSKDAANNNQPDPALTSGDETSLGRAQRQRKGILKNRLQYPLVPQTRGAPELSWCRAATLGHRAVPAASYGRIYAGGGTGSLSQPASRYSSREQLDLLLRRQLSRERLEEAPAPVLRPLSRPGSQECMDAAPGRLEPKDRGSTLPRRQPPRDYPGAMAGRFGSRDALDLGAPREWLSTLPPPRRTRDLDPQPPPLPLSPQRQLSRDPLLPSRPLDSLSRSSNSREQLDQVPSRHPSREALGPLPQLLRAREDSVSGPSHGPSTEQLDILSSILASFNSSALSSVQSSSTPLGPHTTATPSATASVLGPSTPRSATSHSISELSPDSEVPRSEGHS

SEQ ID NO: 173 - protein, 552 aa, Homo sapiens
MQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 174 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 175 - protein, 359 aa, Homo sapiens
MILANVFCLFFFLGEEFYEASPYEPVTSRLSDIFRLASIFSGTGADPVVSAKSNHCLDAAKACNLNDNCKKLRSSYISICNREISPTERCNRRKCHKALRQFFDRVPSEYTYRMLFCSCQDQACAERRRQTILPSCSYEDKEKPNCLDLRGVCRTDHLCRSRLADFHANCRASYQTVTSCPADNYQACLGSYAGMIGFDMTPNYVDSSPTGIVVSPWCSCRGSGNMEEECEKFLRDFTENPCLRNAIQAFGNGTDVNVSPKGPSFQATQAPRVEKTPSLPDDLSDSTSLGTSVITTCTSVQEQGLKANNSKELSMCFTELTTNIIPGSNKVIKPNSGPSRARPSAALTVLSVLMLKLAL

SEQ ID NO: 176 - protein, 331 aa, Homo sapiens
MILANVFCLFFFLGTGADPVVSAKSNHCLDAAKACNLNDNCKKLRSSYISICNREISPTERCNRRKCHKALRQFFDRVPSEYTYRMLFCSCQDQACAERRRQTILPSCSYEDKEKPNCLDLRGVCRTDHLCRSRLADFHANCRASYQTVTSCPADNYQACLGSYAGMIGFDMTPNYVDSSPTGIVVSPWCSCRGSGNMEEECEKFLRDFTENPCLRNAIQAFGNGTDVNVSPKGPSFQATQAPRVEKTPSLPDDLSDSTSLGTSVITTCTSVQEQGLKANNSKELSMCFTELTTNIIPGSNKVIKPNSGPSRARPSAALTVLSVLMLKLAL

SEQ ID NO: 177 - protein, 464 aa, Homo sapiens
MILANVFCLFFFLDETLRSLASPSSLQGPELHGWRPPVDCVRANELCAAESNCSSRYRTLRQCLAGRDRNTMLANKECQAALEVLQESPLYDCRCKRGMKKELQCLQIYWSIHLGLTEGEEFYEASPYEPVTSRLSDIFRLASIFSGTGADPVVSAKSNHCLDAAKACNLNDNCKKLRSSYISICNREISPTERCNRRKCHKALRQFFDRVPSEYTYRMLFCSCQDQACAERRRQTILPSCSYEDKEKPNCLDLRGVCRTDHLCRSRLADFHANCRASYQTVTSCPADNYQACLGSYAGMIGFDMTPNYVDSSPTGIVVSPWCSCRGSGNMEEECEKFLRDFTENPCLRNAIQAFGNGTDVNVSPKGPSFQATQAPRVEKTPSLPDDLSDSTSLGTSVITTCTSVQEQGLKANNSKELSMCFTELTTNIIPGSNKVIKPNSGPSRARPSAALTVLSVLMLKLAL

SEQ ID NO: 178 - protein, 310 aa, Homo sapiens
MTATEALLRVLLLLLAFGHSTYGAECFPACNPQNGFCEDDNVCRCQPGWQGPLCDQCVTSPGCLHGLCGEPGQCICTDGWDGELCDRDVRACSSAPCANNGTCVSLDDGLYECSCAPGYSGKDCQKKDGPCVINGSPCQHGGTCVDDEGRASHASCLCPPGFSGNFCEIVANSCTPNPCENDGVCTDIGGDFRCRCPAGFIDKTCSRPVTNCASSPCQNGGTCLQHTQGQAICFTILGVLTSLVVLGTVGIVFLNKCETWVSNLRYNHMLRKKKNLLLQYNSGEDLAVNIIFPEKIDMTTFSKEAGDEEI

SEQ ID NO: 179 - protein, 383 aa, Homo sapiens
MTATEALLRVLLLLLAFGHSTYGAECFPACNPQNGFCEDDNVCRCQPGWQGPLCDQCVTSPGCLHGLCGEPGQCICTDGWDGELCDRDVRACSSAPCANNGTCVSLDDGLYECSCAPGYSGKDCQKKDGPCVINGSPCQHGGTCVDDEGRASHASCLCPPGFSGNFCEIVANSCTPNPCENDGVCTDIGGDFRCRCPAGFIDKTCSRPVTNCASSPCQNGGTCLQHTQVSYECLCKPEFTGLTCVKKRALSPQQVTRLPSGYGLAYRLTPGVHELPVQQPEHRILKVSMKELNKKTPLLTEGQAICFTILGVLTSLVVLGTVGIVFLNKCETWVSNLRYNHMLRKKKNLLLQYNSGEDLAVNIIFPEKIDMTTFSKEAGDEEI

SEQ ID NO: 180 - protein, 400 aa, Homo sapiens
MVRPLNPRPLPPVVLMLLLLLPPSPLPLAAGDPLPTESRLMNSCLQARRKCQADPTCSAAYHHLDSCTSSISTPLPSEEPSVPADCLEAAQQLRNSSLIGCMCHRRMKNQVACLDIYWTVHRARSLGNYELDVSPYEDTVTSKPWKMNLSKLNMLKPDSDLCLKFAMLCTLNDKCDRLRKAYGEACSGPHCQRHVCLRQLLTFFEKAAEPHAQGLLLCPCAPNDRGCGERRRNTIAPNCALPPVAPNCLELRRLCFSDPLCRSRLVDFQTHCHPMDILGTCATEQSRCLRAYLGLIGTAMTPNFVSNVNTSVALSCTCRGSGNLQEECEMLEGFFSHNPCLTEAIAAKMRFHSQLFSQDWPHPTFAVMAHQNENPAVRPQPWVPSLFSCTLPLILLLSLW

SEQ ID NO: 181 - protein, 373 aa, Homo sapiens
MANTTGEPEEVSGALSPPSASAYVKLVLLGLIMCVSLAGNAILSLLVLKERALHKAPYYFLLDLCLADGIRSAVCFPFVLASVRHGSSWTFSALSCKIVAFMAVLFCFHAAFMLFCISVTRYMAIAHHRFYAKRMTLWTCAAVICMAWTLSVAMAFPPVFDVGTYKFIREEDQCIFEHRYFKANDTLGFMLMLAVLMAATHAVYGKLLLFEYRHRKMKPVQMVPAISQNWTFHGPGATGQAAANWIAGFGRGPMPPTLLGIRQNGHAASRRLLGMDEVKGEKQLGRMFYAITLLFLLLWSPYIVACYWRVFVKACAVPHRYLATAVWMSFAQAAVNPIVCFLLNKDLKKCLRTHAPCWGTGGAPAPREPYCVM

SEQ ID NO: 182 - protein, 1297 aa, Homo sapiens
MAVPSLWPWGACLPVIFLSLGFGLDTVEVCPSLDIRSEVAELRQLENCSVVEGHLQILLMFTATGEDFRGLSFPRLTQVTDYLLLFRVYGLESLRDLFPNLAVIRGTRLFLGYALVIFEMPHLRDVALPALGAVLRGAVRVEKNQELCHLSTIDWGLLQPAPGANHIVGNKLGEECADVCPGVLGAAGEPCAKTTFSGHTDYRCWTSSHCQRVCPCPHGMACTARGECCHTECLGGCSQPEDPRACVACRHLYFQGACLWACPPGTYQYESWRCVTAERCASLHSVPGRASTFGIHQGSCLAQCPSGFTRNSSSIFCHKCEGLCPKECKVGTKTIDSIQAAQDLVGCTHVEGSLILNLRQGYNLEPQLQHSLGLVETITGFLKIKHSFALVSLGFFKNLKLIRGDAMVDGNYTLYVLDNQNLQQLGSWVAAGLTIPVGKIYFAFNPRLCLEHIYRLEEVTGTRGRQNKAEINPRTNGDRAACQTRTLRFVSNVTEADRILLRWERYEPLEARDLLSFIVYYKESPFQNATEHVGPDACGTQSWNLLDVELPLSRTQEPGVTLASLKPWTQYAVFVRAITLTTEEDSPHQGAQSPIVYLRTLPAAPTVPQDVISTSNSSSHLLVRWKPPTQRNGNLTYYLVLWQRLAEDGDLYLNDYCHRGLRLPTSNNDPRFDGEDGDPEAEMESDCCPCQHPPPGQVLPPLEAQEASFQKKFENFLHNAITIPISPWKVTSINKSPQRDSGRHRRAAGPLRLGGNSSDFEIQEDKVPRERAVLSGLRHFTEYRIDIHACNHAAHTVGCSAATFVFARTMPHREADGIPGKVAWEASSKNSVLLRWLEPPDPNGLILKYEIKYRRLGEEATVLCVSRLRYAKFGGVHLALLPPGNYSARVRATSLAGNGSWTDSVAFYILGPEEEDAGGLHVLLTATPVGLTLLIVLAALGFFYGKKRNRTLYASVNPEYFSASDMYVPDEWEVPREQISIIRELGQGSFGMVYEGLARGLEAGEESTPVALKTVNELASPRECIEFLKEASVMKAFKCHHVVRLLGVVSQGQPTLVIMELMTRGDLKSHLRSLRPEAENNPGLPQPALGEMIQMAGEIADGMAYLAANKFVHRDLAARNCMVSQDFTVKIGDFGMTRDVYETDYYRKGGKGLLPVRWMAPESLKDGIFTTHSDVWSFGVVLWEIVTLAEQPYQGLSNEQVLKFVMDGGVLEELEGCPLQLQELMSRCWQPNPRLRPSFTHILDSIQEELRPSFRLLSFYYSPECRGARGSLPTTDAEPDSSPTPRDCSPQNGGPGH

SEQ ID NO: 183 - protein, 760 aa, Homo sapiens
MKEAALICLAPSVPPILTVKSWDTMQLRAARSRCTNLLAASYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 184 - protein, 790 aa, Homo sapiens
MLRGGRRGQLGWHSWAAGPGSLLAWLILASAGAAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 185 - protein, 796 aa, Homo sapiens
MLRGGRRGQLGWHSWAAGPGSLLAWLILASAGAAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 186 - protein, 658 aa, Homo sapiens
MLLKISEITMPGKIFPLKMAHDLDTGSNGLQRYTISSNPHFHVLTRNRSEGRKFPELVLDKPLDREEQPQLRLTLIALDGGSPPRSGTSEIQIQVLDINDNVPEFAQELYEAQVPENNPLGSLVITVSARDLDAGSFGKVSYALFQVDDVNQPFEINAITGEIRLRKALDFEEIQSYDVDVEATDGGGLSGKCSLVVRVLDVNDNAPELTMSFFISLIPENLPEITVAVFSVSDADSGHNQQVICSIENNLPFLLRPSVENFYTLVTEGALDRESRAEYNITITVTDLGTPRLKTQQSITVQVSDVNDNAPAFTQTSYTLFVRENNSPALHIGSVSATDRDSGINAQVTYSLLPPQDPHLPLSSLVSINADNGHLFALRSLDYEALQSFEFRVGATDRGSPALSSEALVRLLVLDANDNSPFVLYPLQNGSAPCTELVPRAAEPGYLVTKVVAVDGDSGQNAWLSYQLLKATELGLFGVWAHNGEVRTARLLSERDAAKHRLVVLVKDNGEPPRSATATLHVLLVDGFSQPYLPLPEAAPAQAQADSLTVYLVVALASVSSLFLFSVLLFVAVRLCRRSRAASVGRYSVPEGPFPGHLVDVSGTGTLSQSYQYKVCLTGGSETNEFKFLKPIMPNFPPQGTEREMEETPTSRNSFPFS

SEQ ID NO: 187 - protein, 794 aa, Homo sapiens
MMQTKVQNKKRQVAFFILLMLWGEVGSESIQYSVLEETESGTFVANLTKDLGLRVGELASRGARVVFKGNRQHLQFDPQTHDLLLNEKLDREELCGSTEPCVLPFQVLLENPLQFFQASLRVRDINDHAPEFPAREMLLKISEITMPGKIFPLKMAHDLDTGSNGLQRYTISSNPHFHVLTRNRSEGRKFPELVLDKPLDREEQPQLRLTLIALDGGSPPRSGTSEIQIQVLDINDNVPEFAQELYEAQVPENNPLGSLVITVSARDLDAGSFGKVSYALFQVDDVNQPFEINAITGEIRLRKALDFEEIQSYDVDVEATDGGGLSGKCSLVVRVLDVNDNAPELTMSFFISLIPENLPEITVAVFSVSDADSGHNQQVICSIENNLPFLLRPSVENFYTLVTEGALDRESRAEYNITITVTDLGTPRLKTQQSITVQVSDVNDNAPAFTQTSYTLFVRENNSPALHIGSVSATDRDSGINAQVTYSLLPPQDPHLPLSSLVSINADNGHLFALRSLDYEALQSFEFRVGATDRGSPALSSEALVRLLVLDANDNSPFVLYPLQNGSAPCTELVPRAAEPGYLVTKVVAVDGDSGQNAWLSYQLLKATELGLFGVWAHNGEVRTARLLSERDAAKHRLVVLVKDNGEPPRSATATLHVLLVDGFSQPYLPLPEAAPAQAQADSLTVYLVVALASVSSLFLFSVLLFVAVRLCRRSRAASVGRYSVPEGPFPGHLVDVSGTGTLSQSYQYKVCLTGGSETNEFKFLKPIMPNFPPQGTEREMEETPTSRNSFPFS

SEQ ID NO: 188 - protein, 937 aa, Homo sapiens
MAGAGGGLGVWGNLVLLGLCSWTGARAPAPNPGRNLTVETQTTSSISLSWEVPDGLDSQNSNYWVQCTGDGGTTETRNTTATNVTVDGLGPGSLYTCSVWVEKDGVNSSVGTVTTATAPNPVRNLTVEAQTNSSIALTWEVPDGPDPQNSTYGVEYTGDGGRAGTRSTAHTNITVDRLEPGCLYVFSVWVGKNGINSSRETRNATTAPNPVRNLHMETQTNSSIALCWEVPDGPYPQDYTYWVEYTGDGGGTETRNTTNTSVTAERLEPGTLYTFSVWAEKNGARGSRQNVSISTVPNAVTSLSKQDWTNSTIALRWTAPQGPGQSSYSYWVSWVREGMTDPRTQSTSGTDITLKELEAGSLYHLTVWAERNEVRGYNSTLTAATAPNEVTDLQNETQTKNSVMLWWKAPGDPHSQLYVYWVQWASKGHPRRGQDPQANWVNQTSRTNETWYKVEALEPGTLYNFTVWAERNDVASSTQSLCASTYPDTVTITSCVSTSAGYGVNLIWSCPQGGYEAFELEVGGQRGSQDRSSCGEAVSVLGLGPARSYPATITTIWDGMKVVSHSVVCHTESAGVIAGAFVGILLFLILVGLLIFFLKRRNKKKQQKPELRDLVFSSPGDIPAEDFADHVRKNERDSNCGFADEYQQLSLVGHSQSQMVASASENNAKNRYRNVLPYDWSRVPLKPIHEEPGSDYINASFMPGLWSPQEFIATQGPLPQTVGDFWRLVWEQQSHTLVMLTNCMEAGRVKCEHYWPLDSQPCTHGHLRVTLVGEEVMENWTVRELLLLQVEEQKTLSVRQFHYQAWPDHGVPSSPDTLLAFWRMLRQWLDQTMEGGPPIVHCSAGVGRTGTLIALDVLLRQLQSEGLLGPFSFVRKMRESRPLMVQTEAQYVFLHQCILRFLQQSAQAPAEKEVPYEDVENLIYENVAAIQAHKLEV

SEQ ID NO: 189 - protein, 1115 aa, Homo sapiens
MAGAGGGLGVWGNLVLLGLCSWTGARAPAPNPGRNLTVETQTTSSISLSWEVPDGLDSQNSNYWVQCTGDGGTTETRNTTATNVTVDGLGPGSLYTCSVWVEKDGVNSSVGTVTTATAPNPVRNLRVEAQTNSSIALTWEVPDGPDPQNSTYGVEYTGDGGRAGTRSTAHTNITVDGLEPGCLYAFSMWVGKNGINSSRETRNATTAHNPVRNLRVEAQTTSSISLSWEVPDGTDPQNSTYCVQCTGDGGRTETRNTTDTRVTVDGLGPGSLYTCSVWVEKDGVNSSVEIVTSATAPNPVRNLTVEAQTNSSIALTWEVPDGPDPQNSTYGVEYTGDGGRAGTRSTAHTNITVDRLEPGCLYVFSVWVGKNGINSSRETRNATTAPNPVRNLHMETQTNSSIALCWEVPDGPYPQDYTYWVEYTGDGGGTETRNTTNTSVTAERLEPGTLYTFSVWAEKNGARGSRQNVSISTVPNAVTSLSKQDWTNSTIALRWTAPQGPGQSSYSYWVSWVREGMTDPRTQSTSGTDITLKELEAGSLYHLTVWAERNEVRGYNSTLTAATAPNEVTDLQNETQTKNSVMLWWKAPGDPHSQLYVYWVQWASKGHPRRGQDPQANWVNQTSRTNETWYKVEALEPGTLYNFTVWAERNDVASSTQSLCASTYPDTVTITSCVSTSAGYGVNLIWSCPQGGYEAFELEVGGQRGSQDRSSCGEAVSVLGLGPARSYPATITTIWDGMKVVSHSVVCHTESAGVIAGAFVGILLFLILVGLLIFFLKRRNKKKQQKPELRDLVFSSPGDIPAEDFADHVRKNERDSNCGFADEYQQLSLVGHSQSQMVASASENNAKNRYRNVLPYDWSRVPLKPIHEEPGSDYINASFMPGLWSPQEFIATQGPLPQTVGDFWRLVWEQQSHTLVMLTNCMEAGRVKCEHYWPLDSQPCTHGHLRVTLVGEEVMENWTVRELLLLQVEEQKTLSVRQFHYQAWPDHGVPSSPDTLLAFWRMLRQWLDQTMEGGPPIVHCSAGVGRTGTLIALDVLLRQLQSEGLLGPFSFVRKMRESRPLMVQTEAQYVFLHQCILRFLQQSAQAPAEKEVPYEDVENLIYENVAAIQAHKLEV

SEQ ID NO: 190 - protein, 679 aa, Homo sapiens
MQVRELPRGEWQTYSSSISHEATACVVDRLRPFTSYKLRLKATNDIGDSDFSSETEAVTTLQDVPGEPPGSVSATPHTTSSVLIQWQPPRDESLNGLLQGYRIYYRELEYEAGSGTEAKTLKNPIALHAELTAQSSFKTVNSSSTSTMCELTHLKKYRRYEVIMTAYNIIGESPASAPVEVFVGEAAPAMAPQNVQVTPLTASQLEVTWDPPPPESQNGNIQGYKIYYWEADSQNETEKMKVLFLPEPVVRLKNLTSHTKYLVSISAFNAAGDGPKSDPQQGRTHQAAPGAPSFLAFSEITSTTLNVSWGEPAAANGILQGYRVVYEPLAPVQGVSKVVTVEVRGNWQRWLKVRDLTKGVTYFFRVQARTITYGPELQANITAGPAEGSPGSPRDVLVTKSASELTLQWTEGHSGDTPTTGYVIEARPSDEGLWDMFVKDIPRSATSYTLSLDKLRQGVTYEFRVVAVNEAGYGEPSNPSTAVSAQVEAPFYEEWWFLLVMALSSLIVILLVVFALVLHGQNKKYKNCSTGKGISTMEESVTLDNGGFAALELSSRHLNVKSTFSKKNGTRSPPRPSPGGLHYSDEDICNKYNGAVLTESVSLKEKSADASESEATDSDYEDALPKHSFVNHYMSDPTYYNSWKRRAQGRAPAPHSVAILLTSNPSAYLSVAPRGSASW

SEQ ID NO: 191 - protein, 2213 aa, Homo sapiens
MARGARPSAAGGGGGGAEPPERAGPGRPRGSPPGRARPSLAPRPGPEPSRPRAAPETSGGDTAGAGRCGGRRAAKLGPGRRGWWALLALQLHLLRALAQDDVAPYFKTEPGLPQIHLEGNRLVLTCLAEGSWPLEFKWMRDDSELTTYSSEYKYIIPSLQKLDAGFYRCVVRNRMGALLQRKSEVQVAYMGSFMDTDQRKTVSQGRAAILNLLPITSYPRPQVTWFREGHKIIPSNRIAITLENQLVILATTTSDAGAYYVQAVNEKNGENKTSPFIHLSIARDVGTPETMAPTIVVPPGNRSVVAGSSETTLECIASARPVEDLSVTWKRNGVRITSGLHSFGRRLTISNPTSADTGPYVCEAALPGSAFEPARATAFLFIIEPPYFTAEPESRISAEVEETVDIGCQAMGVPLPTLQWYKDAISISRLQNPRYKVLASGGLRIQKLRPEDSGIFQCFASNEGGEIQTHTYLDVTNIAPVFTQRPVDTTVTDGMTAILRCEVSGAPKPAITWKRENHILASGSVRIPRFMLLESGGLQIAPVFIQDAGNYTCYAANTEGSLNASATLTVWNRTSIVHPPEDHVVIKGTTATLHCGATHDPRVSLRYVWKKDNVALTPSSTSRIVVEKDGSLLISQTWSGDIGDYSCEIVSEGGNDSRMARLEVIELPHSPQNLLVSPNSSHSHAVVLSWVRPFDGNSPILYYIVELSENNSPWKVHLSNVGPEMTGVTVSGLTPARTYQFRVCAVNEVGRGQYSAETSRLMLPEEPPSAPPKNIVASGRTNQSIMVQWQPPPETEHNGVLRGYILRYRLAGLPGEYQQRNITSPEVNYCLVTDLIIWTQYEIQVAAYNGAGLGVFSRAVTEYTLQGVPTAPPQNVQTEAVNSTTIQFLWNPPPQQFINGINQGYKLLAWPADAPEAVTVVTIAPDFHGVHHGHITNLKKFTAYFTSVLCFTTPGDGPPSTPQLVWTQEDKPGAVGHLSFTEILDTSLKVSWQEPLEKNGIITGYQISWEVYGRNDSRLTHTLNSTTHEYKIQGLSSLTTYTIDVAAVTAVGTGLVTSSTISSGVPPDLPGAPSNLVISNISPRSATLQFRPGYDGKTSISRWIVEGQVGAIGDEEEWVTLYEEENEPDAQMLEIPNLTPYTHYRFRMKQVNIVGPSPYSPSSRVIQTLQAPPDVAPTSVTVRTASETSLRLRWVPLPDSQYNGNPESVGYRIKYWRSDLQSSAVAQVVSDRLEREFTIEELEEWMEYELQMQAFNAVGAGPWSEVVRGRTRESVPSAAPENVSAEAVSSTQILLTWTSVPEQDQNGLILGYKILFRAKDLDPEPRSHIVRGNHTQSALLAGLRKFVLYELQVLAFTRIGNGVPSTPLILERTKDDAPGPPVRLVFPEVRLTSVRIVWQPPEEPNGIILGYQIAYRLASSSPHTFTTVEVGATVRQFTATDLAPESAYIFRLSAKTRQGWGEPLEATVITTEKRERPAPPRELLVPQAEVTARSLRLQWVPGSDGASPIRYFTMQVRELPRGEWQTYSSSISHEATACVVDRLRPFTSYKLRLKATNDIGDSDFSSETEAVTTLQDVPGEPPGSVSATPHTTSSVLIQWQPPRDESLNGLLQGYRIYYRELEYEAGSGTEAKTLKNPIALHAELTAQSSFKTVNSSSTSTMCELTHLKKYRRYEVIMTAYNIIGESPASAPVEVFVGEAAPAMAPQNVQVTPLTASQLEVTWDPPPPESQNGNIQGYKIYYWEADSQNETEKMKVLFLPEPVVRLKNLTSHTKYLVSISAFNAAGDGPKSDPQQGRTHQAAPGAPSFLAFSEITSTTLNVSWGEPAAANGILQGYRVVYEPLAPVQGVSKVVTVEVRGNWQRWLKVRDLTKGVTYFFRVQARTITYGPELQANITAGPAEGSPGSPRDVLVTKSASELTLQWTEGHSGDTPTTGYVIEARPSDEGLWDMFVKDIPRSATSYTLSLDKLRQGVTYEFRVVAVNEAGYGEPSNPSTAVSAQVEAPFYEEWWFLLVMALSSLIVILLVVFALVLHGQNKKYKNCSTGKGISTMEESVTLDNGGFAALELSSRHLNVKSTFSKKNGTRSPPRPSPGGLHYSDEDICNKYNGAVLTESVSLKEKSADASESEATDSDYEDALPKHSFVNHYMSDPTYYNSWKRRAQGRAPAPHRYEAVAGSEAGAQLHPVITTQSAGGVYTPAGPGARTPLTGFSSFV

SEQ ID NO: 192 - protein, 317 aa, Homo sapiens
MAVQGSQRRLLGSLNSTPTAIPQLGLAANQTGARCLEVSISDGLFLSLGLVSLVENALVVATIAKNRNLHSPMYCFICCLALSDLLVSGSNVLETAVILLLEAGALVARAAVLQQLDNVIDVITCSSMLSSLCFLGAIAVDRYISIFYALRYHSIVTLPRARRAVAAIWVASVVFSTLFIAYYDHVAVLLCLVVFFLAMLVLMAVLYVHMLARACQHAQGIARLHKRQRPVHQGFGLKGAVTLTILLGIFFLCWGPFFLHLTLIVLCPEHPTCGCIFKNFNLFLALIICNAIIDPLIYAFHSQELRRTLKEVLTCSW

SEQ ID NO: 193 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 194 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 195 - DNA, 1541 bp, Mus musculus
atggcaccctgcatgctgctcctgctgttggcggccgccctggccccgactcagacccgcgcgggcccacattcgctgaggtatttccacaccgccgtgtcccggcccggcctcgggaagccccggttcatctctgtcggctacgtggacgacacgcagttcgtgcgcttcgacagcgacgcggagaatccgaggtatgagccgcgggtgcggtggatggagcaggtggagcccgagtattgggagcggaacacgcagatcgccaagggcaatgagcagattttccgagtgaacctgaggaccgcgctgcgctactacaaccagagcgcgggcggctctcacacgttccaacggatgtacggctgtgaggtggggtcggactggcgcctcctccgcgggtacgagcagtacgcatacgacggctgcgattacatcgccctgaacgaagacctgaaaacgtggacggcggccgacatggcggcgctgatcaccaaacacaagtgggagcaggctggtgatgcagagagagaccgggcctacctggagggcacgtgcgtggagtggctccgcagatacctgcagctcgggaacgcgacgctgccgcgcacagattccccaaaggcccatgtgacccgtcacagcagacctgaagataaagtcaccctgaggtgctgggccctgggcttctaccctgctgacatcaccctgacctggcagttgaatggggaggagctgacccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacacggtaatcattgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagcgtgaagacagctgcctggagtggacttggtgacagacaatgtcttctcatatctcctgtgacatccagagccctcagttctctttagtcaagtgtctgatgttacctgtgagcctatggactcaatgtgaagaactgtggagcccagtccacccctctacaccaggaccctgtccctgcactgctctgtcttcccttccacagccaaccttgctggttcagccaaacactgagggacatctgtagcctgtcagctccatgctaccctgacctgcaactcctcacttccacactgagaataataatttgaatgtaaccttgattgttatcatcttgacctagggctgatttcttgttaatttcatggattgagaatgcttagaggttttgtttgtttgtttgattgatttgtttttttgaagaaataaatgatagatgaataaacttccagaatttgggtgca

SEQ ID NO: 196 - DNA, 1540 bp, Mus musculus
atggcaccctgcacgctgctcctgctgttggcggccgccctggcccccactcagacccgcgcgggcccacattcgctgaggtatttcgtcaccgccgtgtcccggcccggcctcggggagccccggttcatcgctgtcggctacgtggacgacacgcagttcgtgcgcttcgacagcgacgcggataatccgagatttgagccgcgggcgccgtggatggagcaggaggggccggagtattgggaggagcagacacagagagccaagagcgatgagcagtggttccgagtgagcctgaggaccgcacagagatactacaaccagagcaagggcggctctcacacgttccagcggatgttcggctgtgacgtggggtcggactggcgcctcctccgcgggtaccagcagttcgcctacgacggccgcgattacatcgccctgaacgaagacctgaaaacgtggacggcggcggacacggcggcgctgatcaccagacgcaagtgggagcaggctggtgatgcagagtattacagggcctacctagagggcgagtgcgtggagtggctccgcagatacctggagctcgggaatgagacgctgctgcgcacagattccccaaaggcccatgtgacctatcaccccagatctcaagttgatgtcaccctgaggtgctgggccctgggcttctaccctgctgatatcaccctgacctggcagttgaatggggaggacctgacccaggacatggagcttgtagagaccaggcctgcaggggatggaaccttccagaagtgggcagctgtggtggtgcctcttgggaaggagcagaattacacatgccatgtgcaccataaggggctgcctgagcctctcaccctgagatggaagcttcctccatccactgtctccaacacggtaatcattgctgttctggttgtccttggagctgcaatagtcactggagctgtggtggcttttgtgatgaagatgagaaggaacacaggtggaaaaggagtgaactatgctctggctccaggctcccagacctctgatctgtctctcccagatggtaaagtgatggttcatgaccctcattctctagcgtgaagacagctgcctggagtggacttggtgacagacaatgtcttcacacatctcctgtgacatccagagccctcagttctctttagtcaagtgtctgatgttccctgtgagcctatggactcaaagtgaagaactgtggagcccagtccacccctccacaccagcaccctgtccctgcactgctctgtcttcccttccacagccaaccttgctggttcagccaaacactgggggacatctgcagcctgtcagctccatgctaccctgacctgcagctcctcacttccacactgagaatagtaatttgaatgtaaccttgattgttatcatcttgacctagggctgatttcttgttaatttcatgcttagaggttttgtttgtttgtttgattgttttttttttttgaagaaataaatgatagatgaataaaccgca

SEQ ID NO: 197 - DNA, 2001 bp, Mus musculus
attccagatggggcaatggcactgggaaggctgctcctgctgctggcagccgccctgaccctgaccaaaaccggagcgggctcacactcgctgcggtatttcgagacctcggtgtcccagcccggcttcgggaagccccggttcatctctgtcggctacgtggacgacacgcagtttgtgcgcttcgacagcgacgcgaagaatccgagatatgagctgcgggcgccgtggatggagcaggaggggccggagtattgggagcggaacacacggagagtcaagggcagtgagaagagattccaagagagcctgagcaccctgctcagctactacaaccagagcaagggcggcattcacaccttccagaagttgtctggctgtgatctggggtcagatgggcgccttcaaagcgggtacctgcagttcgcctatgatggccttgattacatcgccctgaatgaagacctggaaacctggacagcagcagatgtggcagctcaggaaacccgacacaagtgggagcaggctggtgctgctgagaaacacaggacctacctggagggcaagtgcctgatgtggctccacagatacctggagctcaggaaggagatgctgctgcgcacagatcccccaaaggcacatgtgactcatcaccccagatctcaaggtgatgtcaccctgaggtgctgggccctgggcttctaccctgctgacatcaccctgacctggcagttgaatggggaggagctgacccagcacatggagcttgtggagaccaggcctgcaggggatggaaccttccagaagtgggcatctgtggtggtgcctcttgggaaggagcagaattacacatgccatgtgtaccatgaggggctgcctgagcccctcaccctgagatgggagcctcctccatacactgtttccaacatggtaatcatagctgttctggttgtccttggagctgtgatagtcattggagctgtggtcatcattggagttatggtgtcttttgtgatgaagaggaggagaaacaaagcatgaagacagctgcctggagtagactgagtgatagccagtgtgttcaggtctctcctgtgacatcaagagtctttagacatcagctttctttagaaaatggtgtctcatgttctctgccacaccaaccccagtagcagccatgtgacaagttgcacagactgaggtatacttcaaaagaaggaggcctcctggaccagttgtggaactcttggccagagtattatattgagtcttagttctcatggcaattgatttattttgttttctcttattccttttcttatttacatgttcctcgtaacctaggctaagatctcatgtgaaagtaatgggttagaaaccatccctagaattggggtcactaacatgaccttatctggggaaaccaacgctttactttagaaattatttacaaaattatataatagtattacacagataaaaacttttccaacaataaaatttcatgtggctacatgtgtgttatcggaggtgggctttggcatgtggtaaagaaagtcttaaataaaaagaattaacatagtttaaatttaagcactgaaaaaaatcaggcattagatgttaaataataattgaccataaagatttattaacatgtcttgaacatatgccagtagccttggatgactgccccactgcttatattcttttagagttgttatacttcaatcatttctactcttcatgatgtcacctgttctgtttgtgattcactgaacacatctttacagaattgtaatgtttgcctgatttttgtgctttcctgtgccaaatgataatgatggtatttgtgattgcttcactggacacatcttcaaagtgttgtaatatttgctcttttaatgtttaaaaacccctgcttgagagctgcaaaatacactcggattcatactgaaaaaaaaaaaaaaaaaaaaaaaaaaaaaaaaaaaaaaaaaaaaaaaa

(19) United States
(12) Patent Application Publication
(10) Pub. No.: US 2020/0385441 A1
(43) Pub. Date: Dec. 10, 2020

(54) IMMUNOTHERAPEUTIC COMPOSITION FOR THE TREATMENT OF CANCER

(71) Applicant: Technion Research & Development Foundation Limited, Haifa (IL)

(72) Inventors: Yoram REITER, Haifa (IL); Netta HAIM, Kibbutz Degania (IL)

(73) Assignee: Technion Research & Development Foundation Limited, Haifa (IL)

(21) Appl. No.: 16/997,963

(22) Filed: Aug. 20, 2020

Related U.S. Application Data
(63) Continuation of application No. PCT/IL2019/050174, filed on Feb. 13, 2019.
(60) Provisional application No. 62/632,452, filed on Feb. 20, 2018.

(51) Int. Cl. C07K 14/74; C07K 16/28; C07K 16/30

(52) U.S. Cl. CPC C07K 14/70539; C07K 16/2833; A61K 38/00; C07K 2317/622; C07K 2319/33; C07K 16/3053

(57) ABSTRACT

The present invention provides compositions and methods for inducing allogenic tumor rejection and, more particularly, but not exclusively, compositions and methods employing fusion proteins comprising an MHC class I HLA amino acid sequence mismatched to the host.

### RELATED APPLICATIONS

This application is a US Continuation of PCT Patent Application No. PCT/IL2019/050174 having International filing date of Feb. 13, 2019, which claims the benefit of priority under 35 USC § 119(e) of U.S. Provisional Patent Application No. 62/632,452 filed on Feb. 20, 2018. The contents of the above applications are all incorporated by reference as if fully set forth herein in their entirety.

### SEQUENCE LISTING STATEMENT

The ASCII file, entitled 83719SequenceListing.txt, created on Aug. 20, 2020, comprising 576,822 bytes, submitted concurrently with the filing of this application is incorporated herein by reference.

### FIELD AND BACKGROUND OF THE INVENTION

The present invention, in some embodiments thereof, relates to a method for treating tumors and, more particularly, but not exclusively, to compositions and methods for eliciting an alloimmune response to tumor cells.

The transfusion of lymphocytes, referred to as adoptive T cell transfer or therapy, is being tested for the treatment of cancer and chronic infections. Adoptive T cell therapy has the potential to enhance antitumor immunity, augment vaccine efficacy, and limit graft-versus-host disease. This form of personalized medicine is now in various early- and late-stage clinical trials. 50-72% response rate has already been achieved in melanoma patients treated with ex vivo expanded autologous tumor infiltrating lymphocytes (TIL). As an alternative to expanding anti-tumor T cells ex vivo, and to broaden the scope of adoptive transfer, the introduction of genes for tumor antigen-specific TCR has been developed as a way of conferring specificity on a patient's own T cells and thus enabling them to attack tumor cells. Using this approach, responses have been observed in melanoma, metastatic colorectal cancer, and synovial cell carcinoma, albeit with some severe autoimmune side effects. Finally, the capacity of CTLs to destroy bulk tumors has been underlined in a most convincing manner by work of Carl June and colleagues using adoptive transfer of autologous T cells in CLL patients after transduction ex vivo with a CD19-specific chimeric antigen receptor (CAR). These are recombinant receptors consisting of a scFv fragment recognizing a tumor antigen, linked to a hinge spacer, a transmembrane domain, and various intracellular signaling domains to allow triggering of T-cell effector function. The CAR used in this study included a signaling element from the 4-1BB co-receptor, which is known to sustain T cells during immune activation. Once in the patients, the T cells underwent marked expansion and were able to delete tumors and deliver sustained complete responses.

While these clinical data underline the potency of CTL against tumor, tailor-made treatments with ex vivo manipulation of effector cells are likely to prove prohibitively expensive on a large scale. An alternative strategy is the idea of activating and re-directing endogenous T cells. One way to do this is to use bispecific antibodies (BsAb) comprising anti-CD3 and anti-tumor antigen moieties. Unfortunately, this is frequently associated with severe toxicity due to the release of a plethora of inflammatory cytokines. Nevertheless, interest in the field has been maintained with a new class of clinical reagent, single-chain bispecific T-cell engagers (BiTEs), which consist of fused scFv domains from an anti-tumor mAb and an anti-CD3 mAb, now in development. The first BiTE, blinatumomab, with specificity for CD19 and CD3 has been trialed as a single agent in non-Hodgkin's lymphoma and ALL with objective clinical responses and acceptable toxicity. Trials with BiTE specific for EpCAM, an antigen widely expressed on human adenocarcinoma and cancer stem cells have recently been initiated.

A refinement of this strategy is to retarget an existing population of CTL of a single specificity, such as for a particular viral antigen. This has been described in WO2003/070752 and WO2007/136778, which disclose the use of an antibody-MHC fusion molecule that carries a viral peptide epitope in order to retarget a predefined oligoclonal population of T cells with viral specificity. This has the great potential advantage in that it avoids the use of anti-CD3 which is non-discriminatory in terms of T-cell recruitment and can trigger cells which are not helpful as effectors but which contribute to the cytokine release syndrome which hamper this approach. Recent studies in mice using the MHC targeting approach applied to the murine system indeed indicated that the MHC targeting approach is less toxic and that mice bearing tumors did not exhibit the cytokine syndrome compared to the bi-specific CD3 construct (King et al. Cancer Immunol Immunother. 62:1093-105, 2013). The toxicity imposed by the CD3 bi-specific approach due to the cytokine burst induced by global T cell recruitment does not only force toxicity issues and administration problems (continuous infusion of very low doses is required to control toxicity) but also limits the maximal tolerated dosage (MTD) of the drug.

Additional background art includes:
WO2001/78768
WO2003/068201
Lev et al. (2004) Proc. Natl. Acad. Sci. USA 101(24):9051-9056
Novak et al. (2007) International Journal of Cancer; 120, 329-36.
Noy et al Molecular Cancer Therapeutics 14, 1327-35 (2015).

### SUMMARY OF THE INVENTION

According to an aspect of some embodiments of the present invention there is provided a method of killing a tumor cell presenting a tumor antigen, the method comprising administering to an individual a composition-of-matter comprising at least one fusion protein comprising a viral MHC-restricted peptide; a human beta-2-microglobulin; an alpha chain of a human MHC class I molecule and a binding domain of an antibody which specifically binds to the tumor antigen, wherein the alpha chain of a human MHC molecule is allogeneic to the individual, so as to elicit an alloimmune response to the tumor cell presenting the antigen, thereby killing the tumor cell.

According to an aspect of some embodiments of the present invention there is provided an article of manufacture comprising a plurality of fusion proteins each packaged in a different package, each fusion protein comprising a viral MHC-restricted peptide; a human beta-2-microglobulin; an alpha chain of a human MHC class I molecule and a binding domain of an antibody which specifically binds to a tumor antigen, wherein the plurality of fusion proteins comprises at least two non-identical fusion proteins having different allogeneic human MHC class I molecule alpha chains.

According to an aspect of some embodiments of the present invention there is provided a composition-of-matter comprising a plurality of fusion proteins, each fusion protein comprising a viral MHC-restricted peptide; a human beta-2-microglobulin; an alpha chain of a human MHC class I molecule and a binding domain of an antibody which specifically binds to a tumor antigen, wherein the plurality of fusion proteins comprises at least two non-identical fusion proteins having different viral MHC-restricted peptides.

According to an aspect of some embodiments of the present invention there is provided an article of manufacture comprising a plurality of fusion proteins each packaged in a different package, each fusion protein comprising a viral MHC-restricted peptide; a human beta-2-microglobulin; an alpha chain of a human MHC class I molecule and a binding domain of an antibody, which specifically binds to a tumor antigen, wherein the plurality of fusion proteins comprises at least two non-identical fusion proteins having different viral MHC-restricted peptides.

According to an aspect of some embodiments of the present invention there is provided a composition-of-matter comprising a plurality of fusion proteins, each fusion protein comprising a viral MHC-restricted peptide; a human beta-2-microglobulin; an alpha chain of a human MHC class I molecule and a binding domain of an antibody which specifically binds to a tumor antigen, wherein the plurality of fusion proteins comprises at least two non-identical fusion proteins having a different binding domain of an antibody which specifically binds to a tumor antigen.

According to an aspect of some embodiments of the present invention there is provided an article of manufacture comprising a plurality of fusion proteins each packaged in a different package, each fusion protein comprising a viral MHC-restricted peptide; a human beta-2-microglobulin; an alpha chain of a human MHC class I molecule and a binding domain of an antibody which specifically binds to a tumor antigen, wherein the plurality of fusion proteins comprises at least two non-identical fusion proteins having a different binding domain of an antibody which specifically binds to a tumor antigen.

According to an aspect of some embodiments of the present invention there is provided a composition-of-matter comprising a plurality of fusion proteins, each fusion protein comprising a viral MHC-restricted peptide; a human beta-2-microglobulin; an alpha chain of a human MHC class I molecule and a binding domain of an antibody which specifically binds to a tumor antigen, wherein the plurality of fusion proteins comprises at least two non-identical fusion proteins having different allogeneic human MHC class I molecule alpha chains.

According to some embodiments of the invention, the alpha chain of the non-identical human MHC class I molecules are selected from the group consisting of HLA-A23, HLA-A32, HLA-A74, HLA-A31, HLA-A80, HLA-A36, HLA-A25, HLA-A26, HLA-A43, HLA-A34, HLA-A66, HLA-A69, HLA-A68, HLA-A29, HLA-B14, HLA-B18, HLA-B27, HLA-B38, HLA-B39, HLA-B41, HLA-B42, HLA-B47, HLA-B48, HLA-B49, HLA-B50, HLA-B52, HLA-B53, HLA-B54, HLA-B55, HLA-B56, HLA-B57, HLA-B58, HLA-B59, HLA-B67, HLA-B73, HLA-B78, HLA-B82, HLA-B81.

According to some embodiments of the invention, the alpha chain of the non-identical human MHC class I molecule has an amino acid sequence at least 95% identical to an amino acid sequence selected from the group consisting of HLA-A23:01:01 (SEQ ID NO: 44), HLA-A32:01:01 (SEQ ID NO: 47), HLA-A74:01:01 (SEQ ID NO: 55), HLA-A31:01:02 (SEQ ID NO: 57), HLA-A80:01:01 (SEQ ID NO: 49), HLA-A36:01 (SEQ ID NO: 56), HLA-A25:01:01 (SEQ ID NO: 45), HLA-A26:01:01(SEQ ID NO: 52), HLA-A43:01(SEQ ID NO: 53), HLA-A34:01:01(SEQ ID NO: 48), HLA-A66:01:01(SEQ ID NO: 50), HLA-A69:01:01(SEQ ID NO: 51), HLA-A68:01:01(SEQ ID NO: 54), HLA-A29:01:01(SEQ ID NO: 46), HLA-B14:01:01(SEQ ID NO: 58), HLA-B18:01:01(SEQ ID NO: 59), HLA-B27:02:01(SEQ ID NO: 60), HLA-B38:01:01(SEQ ID NO: 61), HLA-B39:01:01(SEQ ID NO: 62), HLA-B41:01:01(SEQ ID NO: 63), HLA-B42:01:01(SEQ ID NO: 64), HLA-B47:01:01(SEQ ID NO: 65), HLA-B48:01:01(SEQ ID NO: 66), HLA-B49:01:01(SEQ ID NO: 67), HLA-B50:01:01(SEQ ID NO: 68), HLA-B52:01:01(SEQ ID NO: 69), HLA-B53:01:01(SEQ ID NO: 70), HLA-B54:01:01(SEQ ID NO: 71), HLA-B55:01:01(SEQ ID NO: 72), HLA-B56:01:01(SEQ ID NO: 73), HLA-B57:01:01(SEQ ID NO: 74), HLA-B58:01:01(SEQ ID NO: 75), HLA-B59:01:01(SEQ ID NO: 76), HLA-B67:01:01(SEQ ID NO: 77), HLA-B73:01(SEQ ID NO: 78), HLA-B78:01:01(SEQ ID NO: 79), HLA-B82:01(SEQ ID NO: 80), HLA-B81:01 (SEQ ID NO: 81).

According to some embodiments of the invention, the viral MHC-restricted peptide is 8 or 9 amino acids in length.

According to some embodiments of the invention, the binding domain of the antibody specifically binds to a tumor antigen selected from the group consisting of mesothelin, MCSP and CD25 receptor.

According to some embodiments of the invention, the binding domain of an antibody, which specifically binds to MCSP, has an amino acid sequence as set forth in SEQ ID NO: 27.

According to some embodiments of the invention, the alpha chain of the human MHC class I molecule is an extracellular portion of the alpha chain of the human MHC class I, comprising the human extracellular alpha1, alpha 2 and alpha 3 MHC class I domains.

According to some embodiments of the invention, the viral MHC-restricted peptide, the human beta-2-microglobulin; the alpha chain of the human MHC class I molecule and the binding domain of an antibody which specifically binds to the tumor antigen are N-terminally to C-terminally respectively sequentially translationally fused.

According to some embodiments of the invention, the viral MHC-restricted peptide and the human beta-2-microglobulin are connected by a first peptide linker having an amino acid sequence about 15 amino acids in length.

According to some embodiments of the invention, the amino acid sequence of the first peptide linker is GGGGSGGGGSGGGGS (SEQ ID NO: 16).

According to some embodiments of the invention, the human beta-2-microglobulin and the alpha chain of a human MHC class I molecule are connected via a second peptide linker having an amino acid sequence about 20 amino acids in length.

According to some embodiments of the invention, the amino acid sequence of the second peptide linker is GGGGSGGGGSGGGGSGGGGS (SEQ ID NO: 18).

According to some embodiments of the invention, the alpha chain of the human MHC class I molecule and the binding domain of the antibody which specifically binds to the tumor antigen are connected via a third peptide linker having the amino acid sequence ASGG.

According to some embodiments of the invention, the binding domain of the antibody, which specifically binds to the tumor antigen, is a ScFv fragment of the antibody.

According to some embodiments of the invention, the alpha chain is of a naturally occurring human MHC class I molecule.

According to some embodiments of the invention, the alpha chain is of a non-naturally occurring human MHC class I molecule.

According to some embodiments of the invention, the composition of matter comprises a plurality of the fusion proteins having different allogeneic human MHC molecule alpha chains.

According to some embodiments of the invention, the method of the present invention further comprises determining the MHC class I type of the individual prior to the administering.

According to some embodiments of the invention, selecting the human MHC molecule alpha chain of the fusion protein is based on the MHC class I type of the individual as determined prior to the administering.

According to some embodiments of the invention, the amino acid sequence of the alpha chain of the human MHC class I molecule is no more than 95% identical compared to the amino acid sequences of both of the HLA class I α1-α2 alleles of the individual.

According to some embodiments of the invention, the tumor cell presents mesothelin on its surface.

According to some embodiments of the invention, the binding domain of the antibody specifically binds to mesothelin.

According to some embodiments of the invention, the tumor cell presents MCSP on its surface.

According to some embodiments of the invention, the binding domain of the antibody specifically binds to MCSP.

According to some embodiments of the invention, the method of the invention comprises repeating the administering of the composition of matter.

According to some embodiments of the invention, the method of the invention comprises a plurality of successive cycles of administration, wherein each cycle of administration comprises administering a composition of matter comprising at least one fusion protein comprising a viral MHC-restricted peptide; a human beta-2-microglobulin; an alpha chain of a human MHC class I molecule and a binding domain of an antibody which specifically binds to the tumor antigen, wherein the alpha chain of a human MHC class I molecule is allogeneic to the individual and wherein the alpha chain of the human MHC class I molecule is non-identical to the alpha chain of the human MHC class I molecule of previous cycles of administration.

According to some embodiments of the invention, the cycles of administration are separated by intervals of at least 1 week.

According to some embodiments of the invention, the method further comprises assessing the alloimmune response to the tumor cell in the individual, and commencing a new cycle of administration upon detecting reduced alloimmune response to the alpha chain of the human MHC class I molecule.

According to an aspect of some embodiments of the present invention there is provided an assay for identifying allogeneic human MHC class I alpha chains effective for eliciting an alloimmune response in a subject, the assay comprising:

i) contacting PBMC-derived T cells from the subject with antigen presenting cells from a donor mismatched for MHC class I, thereby activating the T cells;
ii) isolating and culturing the T cells;
iii) contacting the T-cells with
a) a CD19+ B-cell target cell of the subject, and
b) a fusion protein comprising a viral MHC-restricted peptide; a human beta-2-microglobulin; an alpha chain of a human MHC class I molecule HLA-mismatched for the subject and a binding domain of an antibody which specifically binds CD19, and
iv) assaying an immune response of the B-cells,
v) repeating steps i)-iv) using an autologous fusion protein comprising the viral MHC-restricted peptide; the human beta-2-microglobulin and an alpha chain of a human MHC class I molecule HLA-matched for the subject, and
vi) determining effectiveness of the allogeneic human MHC class I alpha chain for eliciting an alloimmune response in the subject by comparing the immune response of the B-cells of the allogeneic with that of the autologous fusion protein, wherein the immune response of the B cells is selected from the group consisting of direct killing of the B-cells, cytokine secretion and T cell activation markers.

According to some embodiments of the invention the alpha chain of the human MHC class I molecule is an extracellular portion of the alpha chain of the human MHC class I, comprising the human extracellular alpha1, alpha 2 and alpha 3 MHC class I domains.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an illustration of antibody-mediated tumor targeting by allogeneic T-cell. Tumor targeting scFv antibody (e.g. anti-MCSP) genetically fused to a mismatched allogeneic foreign (i.e. non-matching) class I single chain MHC molecule carrying a viral peptide (i.e. cellular peptide) recruits allogenic T cells (CTL B) to kill tumor cells presenting the tumor antigen (e.g. MCSP);

FIG. 2 is a schematic representation of protein complexes and peptide/MHC-anti-MCSP fusion protein designs designated: CG (lacking the anti-MCSP scFv binding domain), M15 and BA (left to right is N-terminus to C terminus). β2M-β2 microglobulin; H-2Kb/H-2Kd-MHC alpha chain; His-Histidine tag; BirA-biotinylation substrate tag;

FIG. 3 shows a Western blot of CG or BA fusion molecules expressed in mammalian Expi293 cells, isolated using a His-tag specific antibody. CG fusion molecules (˜50 KDa) lack the anti-MCSP scFv binding domain, BA fusion molecules (˜70 KDa) include the anti-MCSP scFv binding domain. The protein was secreted to the media, His binding by TALON beads was confirmed by incubating 1 ml filtered media with 50 ul beads, washing by centrifugation and eluting with protein sample-buffer (similar data exists for the M15 design);

FIGS. 4A-4D are graphs representing an assay of the MHC folding of CG fusion molecules having different length β2M-MHC linkers [(G4S)₃ or (G4S)₄], using anti-His tag or MHC-fold specific antibodies. MHC folding of CG-biotinylated complexes with 15 or 20 amino acid long β2M-MHC G4S linker was assessed by sandwich ELISA, plates coated with BSA biotin, streptavidin and different concentrations of CG-biotin complex. Peptide-H-2Kd or H-2Kb CGs with 15 amino acid (G4S)₃ (FIGS. 4A and 4B) or 20 amino acid (G4S)₄ (FIGS. 4C and 4D) linkers were incubated with 10 μg/ml mouse anti-His antibody or fold-sensitive (TIB139) antibodies, respectively. Signal of HRP conjugated anti-Mouse was measured by absorbance of colorimetric TMB substrate. Fold-sensitive binding indicates better folding of the fusion proteins with the 20 amino acid (G4S)₄ linkers. Similar results were obtained with BA-biotin fusion molecules;

FIG. 5 shows FACS plots of binding of BA-biotin fusion proteins with 15- or 20-amino acid long β2M-MHC linkers to MCSP-positive B16F10 murine melanoma cells. MCSP-positive (B16F10-MCSP, “C25”) or wild-type MCSP-negative (B16F10) murine melanoma cells were incubated with BA-biotin fusion molecules (BA5 and BA3) having 15 or 20 amino acid length linkers, stained with fold sensitive anti-MHC antibody (TIB139 for H-2Kd or HB79 for H-2Kb) or PE conjugated streptavidin. Note the greater fold-sensitive staining intensity with the 20 amino acid length β2M-MHC linker fusion molecules;

FIGS. 6A and 6B show effective binding of cytotoxic T lymphocytes (CTL) by allogeneic single chain peptide-MHC fusion molecule tetramers. Naïve CD8+ splenocytes from C57BL/6 (H-2Kb) or BalbC (H-2Kd) mice were double stained with H-2Kb (GC1, GC2, GC3) or H-2Kd (GC5, GC7) fusion molecule streptavidin-APC tetramers and PE-conjugated anti-mouse CD8 antibody. FIG. 6A shows the dot plots for two representative mice, showing stronger staining of allogeneic than syngeneic cells. FIG. 6B is a histogram showing percentages of tetramer staining of CD8+ splenocytes, using fusion molecules with 15 or 20 amino acid length β2M-MHC linkers, further confirming greater accuracy of folding of the fusion molecules with 20 amino acid length β2M-MHC linkers;

FIG. 7 contains dot plots of FACS data showing development of subcutaneous MCSP-positive tumors 17 days (two weeks after palpable tumor appearance) following subcutaneous injection of adult C57BL/6 mice with MCSP-positive (“C25”) or MCSP negative (“Wild Type”) B16F10 murine melanoma cells. Data is from two representative tumors and two tissue culture samples maintained for 3 weeks after resection of the tumor;

FIGS. 8A and 8B are graphic representations of T cell population frequencies in the MCSP-positive B16E10 tumors induced in the mice. Comparison of CD44 vs CD62L-gated and CD8 vs CD4 gated FACS dot plots (FIG. 8A) and the frequencies of individual T-cell types (FIG. 8B) did not reveal any significant differences in T-cell profile between the T-cell populations of the MCSP-positive and Wild-type tumors;

FIGS. 9A-9C are graphs showing inhibition of in-vivo tumor growth by allogeneic single chain peptide-MHC fusion molecules. MCSP-positive B16F10 (“C25”) tumors were induced in adult mice by subcutaneous injection of melanoma cells (day 0), and tumor volume (½×W²×L) assessed approx. every three days. Mice were then treated on days 7-11 by i.v. injection of allogeneic MCSP-targeted single chain peptide MHC fusion molecules (M15-12) (FIG. 9C), allogeneic peptide-MHC fusion molecules lacking the single chain scFv anti-MCSP domain (CG-11) (FIG. 9A) or PBS (FIG. 9B). Each plot (e.g. a1, a2, a3 . . . ) represents an individual mouse. Note the significant inhibition of tumor growth, and even tumor rejection in the group treated with allogeneic MCSP-targeted single chain peptide MHC fusion molecules;

FIGS. 10A and 10B are graphs summarizing the results of all treatment groups from the mice treated as in FIGS. 9A-9C. While inclusion of all mice treated with allogeneic MCSP-targeted single chain peptide MHC fusion molecules (M15-12, filled circles) reveals significant inhibition of MCSP-positive tumor growth (FIG. 10A), elimination of the results of a single MS15-12-treated subject (c1) revealed even more significant inhibition of tumor growth by the allogeneic MCSP-targeted single chain peptide MHC (M15-12) fusion molecules;

FIG. 11 is a histogram showing the serum antibody response of mice harboring MCSP-positive melanoma tumors, treated with allogeneic MCSP-targeted single chain peptide MHC fusion molecules. Serum harvested from mice on day 16 after tumor induction (see FIGS. 9A-9C and 10A-10B) was assayed for antibodies to syngeneic MHC-anti-MCSP fusion molecules (BA-5) or allogeneic MHC-anti-MCSP fusion molecules (BA-1) molecules by ELISA. Serum antibodies were detected primarily with the allogeneic (BA-1) rather than syngeneic (BA-5) antigen, indicating immune reaction against the peptide-MHC domains;

FIG. 12 is a histogram showing the effect of added peptide-MHC-fusion molecules (CG-1 complex) to the ELISA reaction detailed in FIG. 11. When the mouse serum was incubated with high concentrations of CG1 complex (peptide-MHC fusion molecule lacking the scFv anti-MCSP domain) during the ELISA assay, significant signal reduction was detected for both the syngeneic (BA-5) and allogeneic (BA-1) assays, indicating that antibodies detected against the syngeneic fusion molecule (BA-5) are directed against the shared domains (His tag, connectors, linkers, etc) of the syngeneic and allogeneic fusion molecules;

FIG. 13 is a schematic depiction of the ex-vivo system for testing human targeted allogeneic rejection alleles. Donor PBMCs are collected from two class I HLA mismatched donors, donor 1 and donor 2. Effector cells (T cells) from donor 1 are activated by culture with allogeneic dendritic cells (cultured from CD14+ donor 2 cells). Activated CD8+ T cells (from donor 1) are then expanded and contacted with freshly isolated syngeneic CD19+ B cells (from donor 1) in the presence of an allogeneic fusion protein comprising anti-CD19 targeting single chain antibody fragment connected to peptide-mismatched (matching donor 2's genotype) HLA molecule, thereby triggering cytotoxic response of the T-cell;

FIGS. 14A-14D are a clustering analysis of class I HLA alleles by protein sequence identity of uncommon versus common class I HLA α1-α2 domains alleles. Two clusters with relative low sequence identity and higher clinical potential can be discerned. Protein sequences of HLA-I α1-2 were aligned by ClastlW2 multiple sequence alignment tool, resulting in a clustering map of relative sequence similarity and sequence identity percentages for every pair of alleles. The resulting percentages are plotted (FIGS. 14A-14D). (FIGS. 14A-14C): All HLA-A and B alleles in rows opposite the uncommon alleles of (FIG. 14A) HLA-A, (FIG. 14B) HLA-B cluster 1 and (FIG. 14C) HLA-B cluster 2, in columns. (FIG. 14D) Protein sequence identity of all HLA-C alleles, rows, against uncommon HLA-A (Top plot), HLA-B cluster 1(middle plot) and HLA-B cluster 2 (bottom plot);

FIGS. 15A-15C demonstrate the high degree of coverage for uncommon HLA-A and B Allo-molecule varieties with less than 95% sequence identity to a patient's genotype.

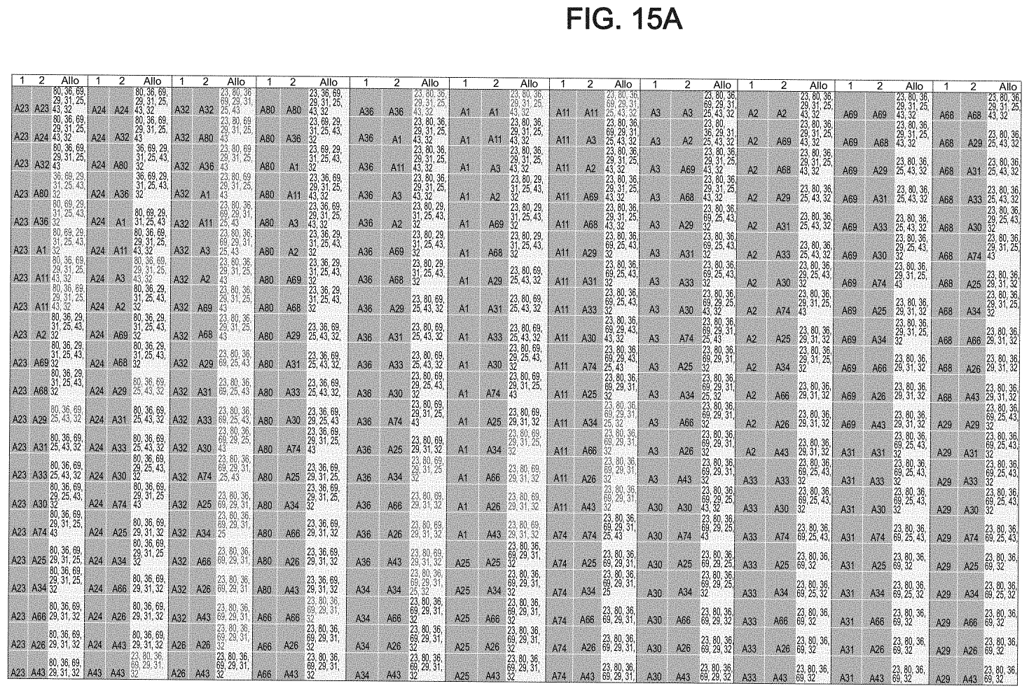

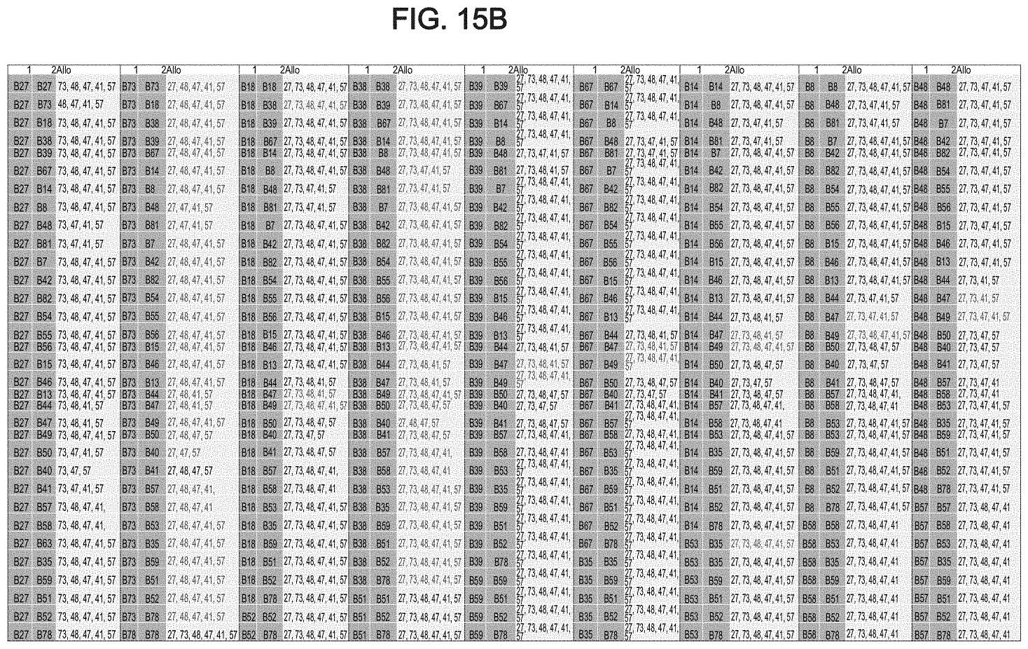

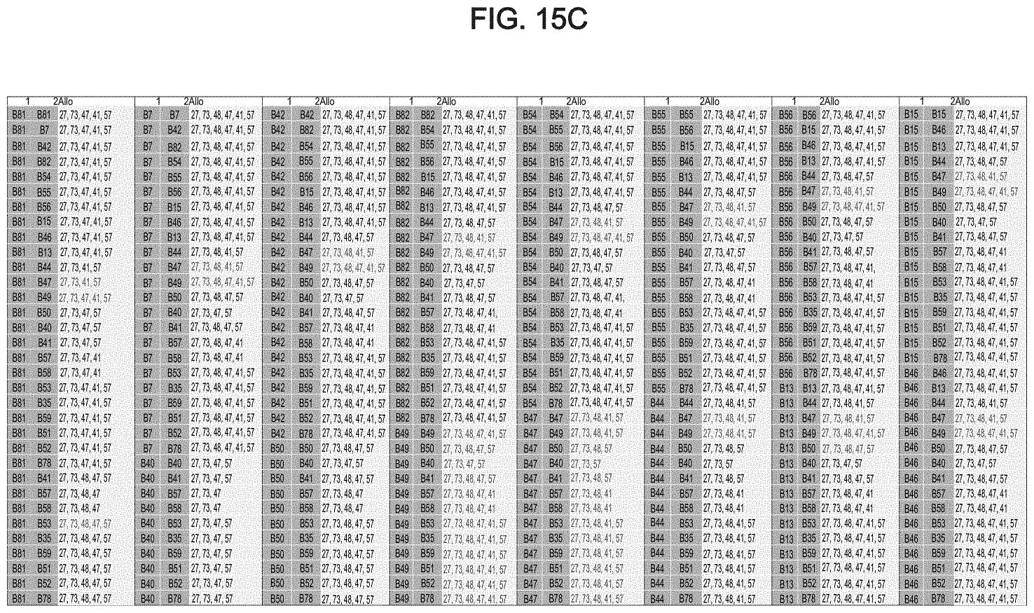

All possible (A) HLA-A or (B-C) HLA-B genotypes of diploid cells with columns and rows representing the two chromosomal sets. Listed for each genotype (columns “1” and “2”) are the uncommon alleles (“Allo”) that can be used for treatment with 91-95% (Red), 86-91% (Black) or less <86% (Blue) α1-2 protein sequence identity between the therapeutic allo-allele and the autologous alleles. FIG. 15A: A sample of 9 uncommon alleles of HLA-A(HLA A*80, 36, 69, 29, 31, 25, 43, 32, 23). FIGS. 15B-15C: A sample of 6 uncommon HLA-B alleles (HLA B*73, 48, 47, 41, 57 from HLA-B cluster 2 and 27 from HLA-B cluster 1.

### DESCRIPTION OF SPECIFIC EMBODIMENTS OF THE INVENTION

The present invention, in some embodiments thereof, relates to compositions and methods for inducing allogenic tumor rejection and, more particularly, but not exclusively, to compositions and methods employing fusion proteins comprising an MHC class I HLA amino acid sequence mismatched to the host.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

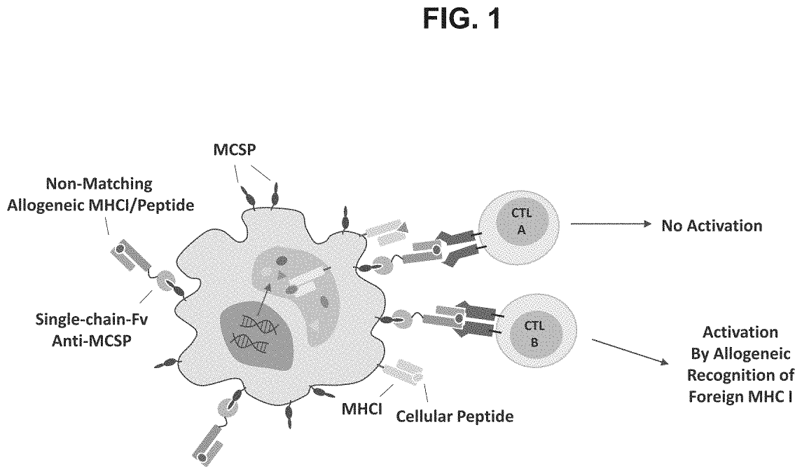

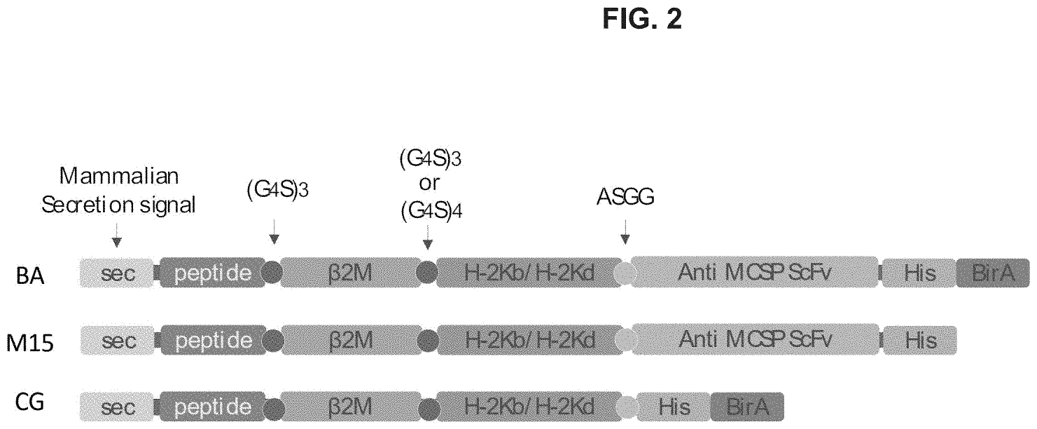

The potency of immunotherapies targeting endogenous tumor antigens is hindered by immune tolerance. To overcome immunological tolerance the inventors have previously shown that a fusion protein comprising a tumor targeting antibody fused to a class I human HLA molecule that carries a potent immunogenic peptide (e.g. a viral-derived epitope, see FIGS. 1 and 2) can recruit potent effector CD8+ T cells to the tumor site: a single chain antibody fused to a human MHC (HLA 2A) complex with viral peptides recruits CD8+ T cells and inhibits the growth human cancer xenografts in nude mice receiving specific CD8 T cell lines by adoptive cell transfer (Lev et al. (2004) Proc. Natl. Acad. Sci. USA 101(24):9051-9056; Novak et al. (2007) International Journal of Cancer 120, 329-36 Noy et al (2015) Molecular Cancer Therapeutics 14, 1327-35).

While conceiving embodiments of the present invention and reducing it to practice, the instant inventors have now developed compositions and methods for treatment of tumors based on allogeneic rejection. In allogeneic rejection of transplants, the immune system reacts to foreign cells following organ transplantations between genetically mismatched individuals. Unlike syngeneic (e.g. autologous) transplantation, where the donor and the recipient share the same gene variants (alleles) for the Major Histocompatibility Complexes (MHC), allogeneic transplantation requires an allelic mismatch between donor and recipient MHC genes.

The MHC class I complexes are found on the outer membranes of every nucleated cell in the body; one of their functions is to bind peptides (processed protein fragments representing the proteome of the cell) and present them on the outside to CD8 cytotoxic T cells. When a cell is infected or transformed, abnormal proteins are produced by the cell and as a result MHC I complexes present viral or mutated peptides, consequently activating cytotoxic CD8 T cells bearing T Cell Receptors (TCRs) that can specifically recognize these peptides in an MHC context and kill the cell. In allogeneic transplantation, the CD8 T cells of the host can promiscuously recognize the foreign MHCs as an infected or transformed cell, regardless of the origin of the peptide presented by the MHC, killing it and rejecting the transplanted organ. These promiscuous memory CD8 T cells are initially activated by a pathogenic peptide-syngeneic MHC complex but can also recognize peptide-allogeneic MHC complexes with a single T cell receptor.

The instant inventors have now shown that a therapeutic agent comprising a tumor-homing module fused to a functional domain of an allogeneic (recipient mismatched) MHC I molecule can selectively render tumor cells sensitive to allogeneic rejection (see Example 8). The allogenic fusion protein comprises a tumor-homing module having a binding domain (e.g. Fab, single-chain variable fragment (scFv), linear antibody, Fv or any other protein sequence that can fold so that the binding domain of the monoclonal antibody is formed) specifically binding a tumor antigen, genetically fused to a functional T cell recruitment or engagement domain comprising the alpha1, alpha2 and alpha 3 domains of an engineered single alpha chain MHC class I HLA molecule of an allele mismatched to the acceptor/recipient MHC class I HLA and a self or influenza-derived peptide to elicit site-specific allogeneic T cell recruitment and response localized at the tumor site, thus inducing a site- and tumor-specific tumor rejection reaction and thereby, circumventing immune tolerance.

Another allogeneic rejection mechanism involves the activation of allo-reactive B cells. The instant inventors have uncovered that fusion proteins MHC class I HLA molecule of an allele mismatched to the acceptor/recipient MHC class I HLA also induce a potent humeral and cellular immune response when transplanted (see Example 11).

Thus, according to one aspect of the invention there is provided a method of killing a tumor cell presenting a tumor antigen, the method comprising administering to an individual a composition of matter comprising at least one fusion protein comprising a viral MHC-restricted peptide; a human beta-2-microglobulin; an alpha chain of a human MHC class I molecule and a binding domain of an antibody which specifically binds to the tumor antigen, wherein the alpha chain of a human MHC molecule is allogeneic to the individual, so as to elicit an alloimmune response to the tumor cell presenting the antigen, thereby killing the tumor cell.

In the cell, the MHC alpha chain comprises a functional, extracellular portion, a transmembrane component and a cytoplasmic “tail”. In specific embodiments, the alpha chain of the human MHC class I molecule is an extracellular portion of the human MHC alpha chain, comprising the human alpha1, alpha2 and alpha3 MHC class I domains.

In specific embodiments the viral MHC-restricted peptide, the human beta-2-microglobulin; the alpha chain of said human MHC class I molecule and the binding domain of an antibody which specifically binds to the tumor antigen of the composition of matter of the invention are N-terminally to C-terminally respectively sequentially translationally fused. In other specific embodiments, the viral MHC-restricted peptide and the human beta-2-microglobulin are connected by a first peptide linker having an amino acid sequence about 15 amino acids in length.

In yet other specific embodiments, the human beta-2-microglobulin and the alpha chain of a human MHC class I molecule are connected via a second peptide linker having an amino acid sequence about 20 amino acids in length. In still other specific embodiments, the alpha chain of the human MHC class I molecule and the binding domain of said antibody, which specifically binds to the tumor antigen, are connected via a third peptide linker having the amino acid sequence ASGG.

In one embodiment, the first peptide linker has the amino acid sequence GGGGSGGGGSGGGGSGGGGS (SEQ ID NO: 18). In another embodiment, the second peptide linker has the amino acid sequence GGGGSGGGGSGGGGS (SEQ ID NO: 16). In another embodiment, the third peptide linker has the amino acid sequence ASGG. As used herein, “first peptide linker”, “second peptide linker” refer to peptides composed of a monomeric peptide whose amino acid sequence is GXGGS or a multimer thereof, wherein X may be any amino acid. These peptide linkers may be a multimer of 2-10 of such monomeric peptide. In any such multimer, each monomeric peptide may be the same as or different from other monomeric peptide in the multimer depending on the identity of amino acid X. In one embodiment, X in the monomeric peptide is the amino acid valine (V). In another embodiment, X in the monomeric peptide is the amino acid glycine (G). In specific embodiments, the peptide linker comprises a multimer of three or four monomeric peptides, particularly a multimer of three monomeric peptides in which the most N-terminal X is the amino acid V, and the second and third X are the amino acid G.

In specific embodiments, the composition of matter of the invention comprises at least one fusion protein. As used herein, the term “fusion protein” refers to a polypeptide including at least two segments linked together by peptide bonds (e.g. translationally fused), each of which shows a high degree of amino acid identity to a peptide moiety that (1) occurs in nature, and/or (2) represents a functional domain of a polypeptide. Typically, a polypeptide containing at least two such segments is considered to be a fusion protein if the two segments are moieties that (1) are not included in nature in the same peptide, and/or (2) have not previously been linked to one another in a single polypeptide, and/or (3) have been linked to one another through action of the hand of man.

In other embodiments, the component sequences of the fusion protein are translationally fused. As used herein, the phrases “translationally fused” and “in frame” are interchangeably used to refer to polypeptides encoded by polynucleotides, which are covalently linked to form a single continuous open reading frame spanning the length of the coding sequences of the linked polynucleotides. Such polynucleotides can be covalently linked directly or preferably indirectly through a spacer or linker region encoding a linker peptide. “Sequentially translationally fused” relates to the spatial order of the component polypeptide sequences (segments) comprising a fusion protein. As used herein, the phrase “N-terminally to C-terminally respectively translationally fused” is used herein to refer to the respective spatial order of the component sequences (segments) of the fusion protein, beginning at the amino (“N-”) terminus of the fusion protein and proceeding to the carboxy (“C-”) terminus, with the C-terminus of each of the component sequences (segments) fused to the N-terminus of the adjacent sequence (segment), for example, as illustrated in FIG. 2 (“N-terminus” is on the left and “C-terminus” is on the right of each of the represented fusion proteins).

As used herein, the term “MHC-restricted peptide” or “MHC-restricted antigen” refers to a cell surface peptide or cell surface antigen displayed by an MHC molecules or potentially displayed by an MHC molecule. T lymphocyte receptors, unlike antibodies, do not recognize native antigens but rather recognize cell-surface displayed complexes comprising an intracellularly processed fragment of a protein or lipid antigen in association with a specialized antigen-presenting molecule (APM): major histocompatibility complex (MHC) for presentation of peptide antigens; and CD1 for presentation of lipid antigens, and to a lesser extent, peptide antigens. Peptide antigens displayed by MHC molecules and lipid antigens displayed by CD1 molecules have characteristic chemical structures are referred to as MHC-restricted peptides and CD1 restricted lipids, respectively.

As used herein, the term “MHC” refers to Major Histocompatibility Complex, and “MHC molecule” refers to Major Histocompatibility Complex molecule. Major histocompatibility complex molecules are highly polymorphic, comprising more than 40 common alleles for each individual gene. “Classical” MHC molecules are divided into two main types, class I and class II, having distinct functions in immunity.

The class I MHC molecule is a heterodimer composed of a 46-kDa heavy chain, which is non-covalently associated with the 12-kDa light chain β-2 microglobulin. Major histocompatibility complex class I (MHC class I) molecules are expressed on the surface of virtually all cells in the body and are dimeric molecules composed of a transmembrane alpha chain, comprising the peptide antigen binding cleft, and a smaller extracellular chain termed beta-2-microglobulin. MHC class I molecules present 9- to 11-amino acid residue peptides (“MHC-restricted peptide” or “MHC-presented peptide”) derived from the degradation of cytosolic proteins by the proteasome, a multi-unit structure in the cytoplasm. Cleaved peptides are transported into the lumen of the endoplasmic reticulum (ER) by TAP where they are bound to the groove of the assembled class I molecule, and the resultant MHC/antigen complex is transported to the cell membrane to enable antigen presentation to T lymphocytes.

Major histocompatibility complex class II molecules are expressed on a restricted subset of specialized antigen-presenting cells (APCs) involved in T lymphocyte maturation and priming. Such APCs in particular include dendritic cells and macrophages, cell types which internalize, process and display antigens sampled from the extracellular environment. Unlike MHC class I molecules, MHC class II molecules are composed of an alpha-beta transmembrane dimer whose antigen binding cleft can accommodate peptides of about 10 to 30, or more, amino acid residues.

The three-dimensional structure of MHC class I and II molecules are very similar but important differences exist. MHC class I alpha chain is encoded in the gene complex termed the major histocompatibility complex (MHC), and its extracellular portion comprises three domains, alpha1, alpha2 and alpha3. Thus, as used herein, the phrase “alpha chain of a human MHC class I molecule” refers to an MHC molecule comprising human class I alpha chain domains, alpha1, alpha2 and alpha3.

The beta2microglobulin chain is not encoded in the MHC gene and consists of a single domain, which together with the alpha3 domain of the alpha chain make up a folded structure that closely resembles that of the immunoglobulin. The a1 and a2 domains pair to form the peptide binding cleft, consisting of two segmented alpha helices lying on a sheet of eight beta-strands.

According to specific embodiments of the present invention, the alpha chain of the human MHC molecule is allogeneic to the individual, eliciting an alloimmune response. As used herein, the term “allogeneic” refers to a mismatch between the amino acid sequence of a host's (e.g. the individual's) MHC complex molecule and that of the alpha chain of the human MHC molecule comprised within the fusion protein. In specific embodiments, the mismatch between the individual's MHC molecule and that of the alpha chain of the human MHC molecule comprised within the fusion protein is sufficient to elicit an alloimmune response.

Optimal mismatching between the host MHC class I alleles and those of the allogeneic fusion protein MHC class I molecule can be a degree of difference sufficient to produce an allogeneic T cell response that is not so strong as to cause a cytokine storm, but not too weak that the response fails to cause rejection of the tumor. According to some embodiments, selection of the alpha chain of the allogeneic MHC class I molecule of the fusion protein of the invention is based on recognition of uncommon human Class I HLA alleles.

### Employing Human Uncommon Allogeneic Class I HLA Alleles for Targeted Allogeneic Cancer Rejection Strategy

The human genome contains three MHC class I α chain genes; A, B and C, each with its own degree of polymorphism. The HLA B gene has the greatest number of different alleles, which give rise to different amino acid sequences, the HLA A gene has intermediate number and HLA C gene has the smallest number of alleles. Furthermore, distribution of alleles in various populations is diverse, each human population having its common and uncommon alleles, certain alleles can be very common in an isolated population but virtually absent in another. (HLA amino acid sequences can be found at the Kabat data base, at htexttransferprotocol://immuno.bme.nwu.edu. Further information concerning MHC haplotypes can be found in Paul, B. Fundamental Immunology Lippincott-Rven Press.)

However, several common alleles are highly represented in many populations:

TABLE 1
Common and Uncommon HLA Class I Alleles

| UNCOMMON ALLELES | COMMON ALLELES |
|---|---|
| A*23; A*32; A874; A*31; A*80; A*36; A*25; A*26; | A*24; A*03; A*01; A*11; A*33; A*30; |
| A*43; A*34; A*66; A*68; A*69; A*29; B*14; B*18; | A*02; B*07; B*08; B*13; B*15; B*35; |
| B*27; B*38; B*39; B*41; B*42; B*47; B*48; B*49; | B*40; B*44; B*46; |
| B*50; B*52; B*53; B*54; B*55; B*56; B*57; B*58; | B*51; C*01; C*03; C*04; C*06; C*07; C*12 |
| B*59; B*67; B*73; B*78; B*82; B*81; C*02; C*05; | |
| C*08; C*14; C*15; C*16; C*17; C*18 | |

(Based on aggregate data visualization on the “allelefrequencies” website, current to December 2017)

Most of the amino-acid polymorphism found in HLA class I genes is located in the α1 and α2 domains, the α3 sequence being more highly conserved. Due to the importance of the α1 and α2 domains in the interaction with the TCR complex, both by affecting the peptide binding capacity and via direct interaction, differences in these two domains between individuals are essential for the elicitation of allogeneic CTL activity. Of the CTL population in naïve animals, 1-10% were reported to recognize allogeneic alleles independent of the identity of the presented peptide, varying with the specific allo-allele and method of measurement.

In the HLA class I system, each allele has many sub-alleles that differ from each other in the DNA coding sequence, differences that may or may not result in a small change in the amino acid sequence. In most cases, these small differences between sub-alleles have little or no effect on the peptide binding capacity and are less likely to produce significant allogeneic CTL activity. Thus, in some embodiments, the degree of allogenicity is analyzed for a representative sub-allele of each allele. Some HLA class I sub-alleles suitable for determining degree of allogenicity are listed in Table 2:

TABLE 2
Exemplary HLA Class I Allele Subtypes

A*01.01.01; A*02.01.01; A*03.01.01; A*11.01.01; A*23.01.01; A*24.02.01; A*25.01.01;
A*26.01.01; A*29.01.01; A*30.01.01; A*31.01.02; A*32.01.01; A*33.01.01; A*33.01.01;
A*34.01.01; A*36.01; A*43.01; A*66.01.01; A*68.01.01; A*69.01.01; A*74.01.01;
A*80.01.01; B*07.02.01; B*08.01.01; B*13.01.01; B*14.01.01; B*15.01.01; B*18.01.01;
B*27.02.01; B*35.01.01; B*38.01.01; B*39.01.01; B*40.01.01; B*41.01.01; B*42.01.01;
B*44.02.01; B*46.01.01; B*47.01.01; B*48.01.01; B*49.01.01; B*50.01.01; B*51.01.01;
B*52.01.01; B*53.01.01; B*54.01.01; B*55.01.01; B*56.01.01; B*57.01.01; B*58.01.01;
B*59.01.01; B*67.01.01; B*73.01; B*78.01.01; B*81.01; B*82.01; C*01.02.01; C*02.02.01;
C*03.02.01; C*04.01.01; C*05.01.01; C*06.02.01; C*07.01.01; C*08.01.01; C*12.02.01;
C*14.02.01; C*15.02.01; C*16.01.01; C*17.01.01 C*18.01

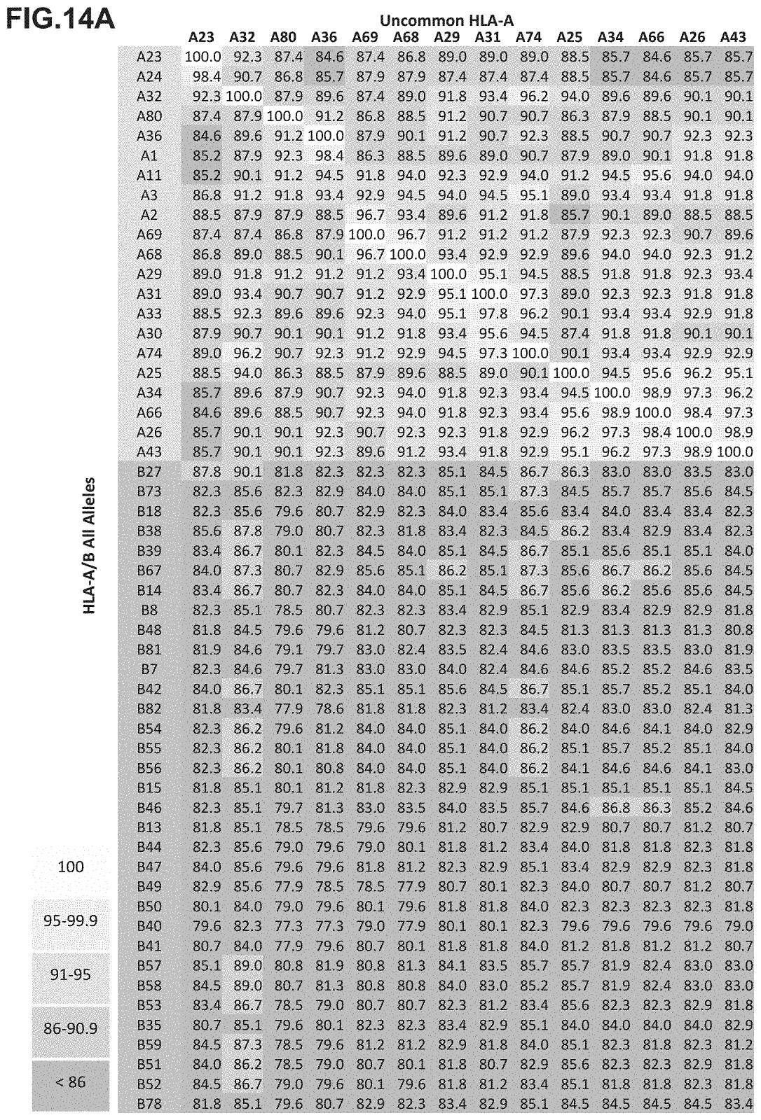

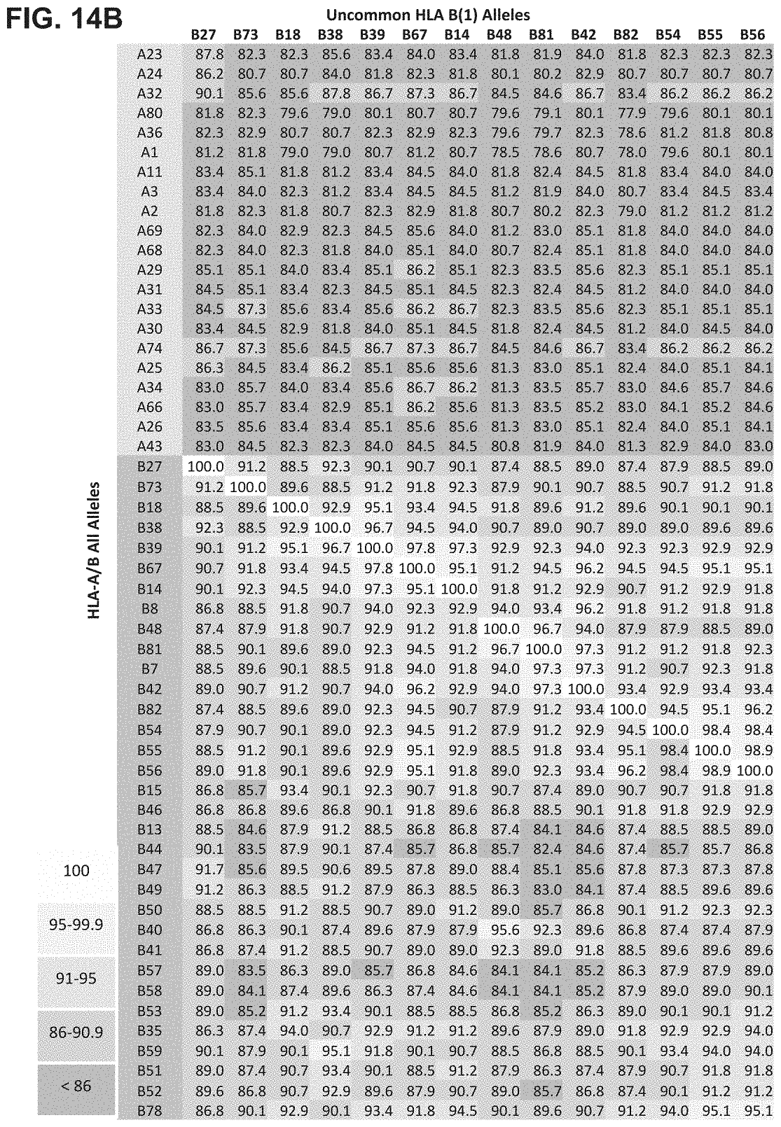

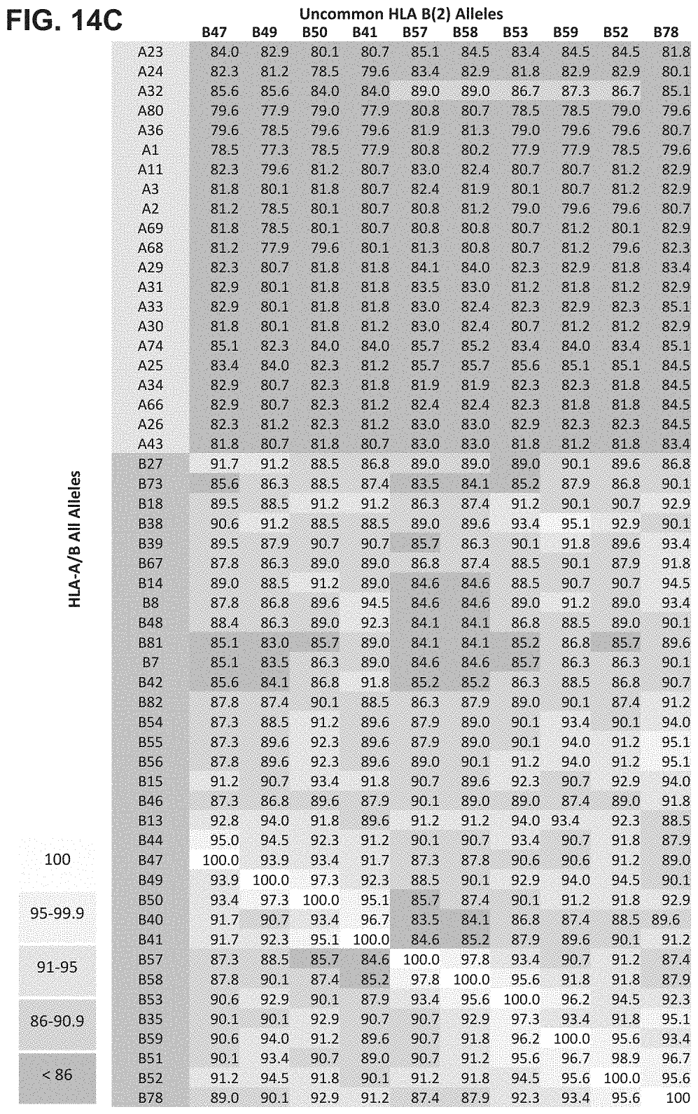

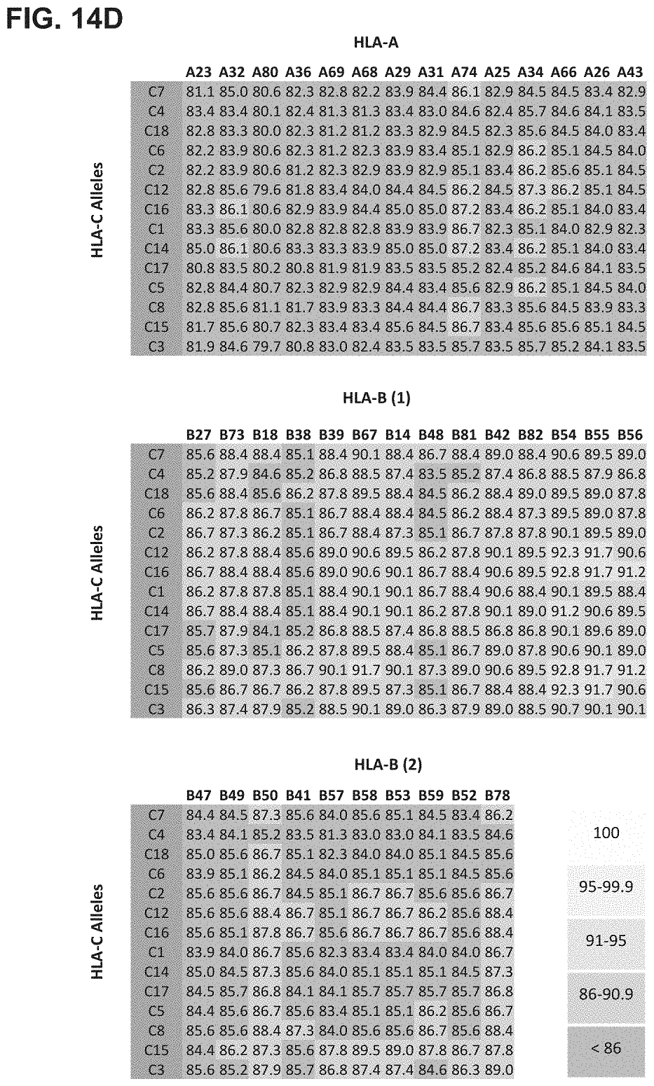

In some embodiments, selection of suitable mismatched HLA class I alleles is based on first determining the sequence diversity of HLA class I alleles by aligning the α1 (AA₍₂₅₋₉₀₎) and a2 (AA₍₉₁₋₁₈₂₎) sequences for each allele using a multiple sequence alignment tool (e.g. Clustal Omega) and building a phylogenic tree and a table of sequence identity percentages between the different alleles. These data, combined with lists of uncommon alleles, such as Table 1 hereinabove, can then be used to determine the sequence clustering of inter-allele identity in α1-α2 protein sequence of uncommon alleles vs. all alleles (see, for example, FIGS. 14A-14D).

It will be appreciated that, in some embodiments, uncommon HLA class I alleles with sequence diversity that will cover a large proportion of the population expressing the common HLA alleles are desirable for designing the allogeneic treatment. By clustering of the alleles into four regions according the sequence similarity tree, the instant inventors have revealed that each of the HLA A (FIG. 14A) and HLA-C (FIG. 14D) could be clustered into its own branch; however, HLA B is divided in to two separate branches, indicated as HLA B (1) (FIG. 14B) and HLA B (2)(FIG. 14C).

Comparison of sequence identity revealed that HLA A uncommon alleles, for the most part, are less than 86% identical to the HLA B and C alleles. Thus, in some embodiments, the allogeneic human MHC alpha chain is selected mismatched to the HLA A genotype of a patient, and not according to the HLA B or HLA C genotype f the individual (e.g. patient). Further, in some embodiments, wherein the individual's (e.g. patient's) HLA A genotype includes HLA A*24, the allogeneic human MHC alpha chain is selected from the uncommon HLA A*23 and 32 alleles.

Comparing the HLA B (2) uncommon alleles with alleles of both HLA A and C revealed that they are mostly less than 86% different from both HLA A and C, thus, in some embodiments, the allogeneic human MHC alpha chain is selected mismatched to the HLA B genotype of a patient, and not according to the HLA A or HLA C genotype of the individual (e.g. patient). Importantly, because the HLA B (2) cluster is composed of two smaller clusters, there is a higher degree of internal sequence difference in HLA B (2) in comparison to HLA A, so that fewer HLA B (2) alleles will be required to cover all genotypes.

Further comparison revealed that the HLA C gene has relative low polymorphism and high degree of sequence identity, thus, in specific embodiments, the allogeneic human MHC alpha chain is selected from the uncommon HLA A and HLA B (2) alleles.

In some embodiments, the human MHC class I molecule alpha chain of the fusion protein of the present invention is selected based upon the MHC class I type of the individual (e.g. patient) as determined, prior to administration of the composition of matter of the present invention.

It will further be appreciated that the degree of mismatch between the MHC class I molecule of the fusion protein and those of the individual (e.g. patient) needs to be significant enough to elicit an allogeneic response powerful enough to seriously damage or kill the targeted tumor cells. In some embodiments, allogeneic fusion protein molecules with HLA class I α1-α2 protein sequence identity of less than (<) 95% compared to both of the patient alleles are considered different enough for eliciting allogeneic response for treatment. In specific embodiments, the allogeneic fusion protein molecules selected have HLA class I α1-α2 protein sequence identity of less than 95%, less than 94%, less than 93%, less than 92%, less than 91%, less than 90%, less than 89%, less than 88%, less than 87%, less than 86%, less than 85%, less than 84%, less than 83%, less than 82% or less than 80%, compared to both of the patient alleles. In yet other embodiments, the selected allogeneic fusion protein molecules have HLA class I α1-α2 protein sequence identity in the range of 91% to less than 95%, 89% to less than 93%, 88% to less than 92%, 86% to less than 91%, and less than 86% compared to both of the patient alleles. Exemplary combinations of HLA A allo-alleles treatments using a sample of 9 uncommon alleles (HLA A*80, 36, 69, 29, 31, 25, 43, 32, 23) and for HLA B a sample of 6 alleles (HLA B*73, 48, 47, 41, 57 from HLA B (2) and 27 from HLA B (1)) are shown in FIG. 15A (HLA-A) and 15B (HLA-B), respectively.

Thus, in some embodiments, the alpha chain of the non-identical (mismatched) human MHC class I molecule is selected from the group consisting of HLA-A23, HLA-A32, HLA-A74, HLA-A31, HLA-A80, HLA-A36, HLA-A25, HLA-A26, HLA-A43, HLA-A34, HLA-A66, HLA-A69, HLA-A68, HLA-A29, HLA-B14, HLA-B18, HLA-B27, HLA-B38, HLA-B39, HLA-B41, HLA-B42, HLA-B47, HLA-B48, HLA-B49, HLA-B50, HLA-B52, HLA-B53, HLA-B54, HLA-B55, HLA-B56, HLA-B57, HLA-B58, HLA-B59, HLA-B67, HLA-B73, HLA-B78, HLA-B82 and HLA-B81. In specific embodiments, the alpha chain of the non-identical (mismatched) human MHC class I molecule has an amino acid sequence at least 95% identical to, at least 96% identical to, at least 97% identical to, at least 98% identical to, at least 99% identical to or 100% identical to an amino acid sequence selected from the group consisting of HLA-A23:01:01 (SEQ ID NO: 44), HLA-A32:01:01 (SEQ ID NO: 47), HLA-A74:01:01 (SEQ ID NO: 55), HLA-A31:01:02 (SEQ ID NO: 57), HLA-A80:01:01 (SEQ ID NO: 49), HLA-A36:01 (SEQ ID NO: 56), HLA-A25:01:01 (SEQ ID NO: 45), HLA-A26:01:01(SEQ ID NO: 52), HLA-A43:01(SEQ ID NO: 53), HLA-A34:01:01(SEQ ID NO: 48), HLA-A66:01:01(SEQ ID NO: 50), HLA-A69:01:01(SEQ ID NO: 51), HLA-A68:01:01(SEQ ID NO: 54), HLA-A29:01:01(SEQ ID NO: 46), HLA-B14:01:01(SEQ ID NO: 58), HLA-B18:01:01(SEQ ID NO: 59), HLA-B27:02:01(SEQ ID NO: 60), HLA-B38:01:01(SEQ ID NO: 61), HLA-B39:01:01(SEQ ID NO: 62), HLA-B41:01:01(SEQ ID NO: 63), HLA-B42:01:01(SEQ ID NO: 64), HLA-B47:01:01(SEQ ID NO: 65), HLA-B48:01:01(SEQ ID NO: 66), HLA-B49:01:01(SEQ ID NO: 67), HLA-B50:01:01(SEQ ID NO: 68), HLA-B52:01:01(SEQ ID NO: 69), HLA-B53:01:01(SEQ ID NO: 70), HLA-B54:01:01(SEQ ID NO: 71), HLA-B55:01:01(SEQ ID NO: 72), HLA-B56:01:01(SEQ ID NO: 73), HLA-B57:01:01(SEQ ID NO: 74), HLA-B58:01:01(SEQ ID NO: 75), HLA-B59:01:01(SEQ ID NO: 76), HLA-B67:01:01(SEQ ID NO: 77), HLA-B73:01(SEQ ID NO: 78), HLA-B78:01:01(SEQ ID NO: 79), HLA-B82:01(SEQ ID NO: 80), HLA-B81:01 (SEQ ID NO: 81).

In some embodiments, the human MHC alpha chain of fusion proteins having human MHC class I alpha chains mismatched to those of the individual (e.g. patient) is a naturally occurring human MHC class I molecule, i.e. having an alpha-chain amino acid sequence found in nature or highly homologous (at least 95%, 96%, 97%, 98%, or 100% identical) thereto. Also contemplated are human MHC alpha chain of fusion proteins having human MHC class I alpha chains mismatched to those of the individual (e.g. patient) which are non-naturally occurring human MHC class I molecules, i.e. having an alpha-chain amino acid sequence not found in nature or having fewer than 95% amino acid identity to the human MHC alpha chain (alpha a1, alpha a2 and alpha a3 domains). Non-naturally occurring, or synthetic MHC molecules, and methods for their production are described, inter alia, in Tuchscherer et al, Protein Science 1992, 1:1377-86 and US Patent Application 20030068363 to Clark et al.

An alloimmune response occurs when CD8 T cells of the host “promiscuously” identify other unsimilar (e.g. foreign) MHCs as belonging to an infected or transformed cell, and mount a T-cell response against the cell or cells bearing the allogeneic MHCs, regardless of the origin of the peptide presented by the MHC. The T cell response can include, but is not limited to, T-cell proliferation, T-cell activation, T-cell differentiation, and the like.

Another allogeneic rejection mechanism involves the activation of allo-reactive B-cells. Thus, an alloimmune response can also be or include a B-cell response. B-cells responding to unsimilar MHCs, or to fusion proteins comprising mismatched MHC molecules, via binding of antigens at the B-cell receptor, can react by proliferating, and initiating activation, resulting in differentiation to short-lived plasmablasts, memory B-cells, long-lived plasma cells, and the like, responsible for production of antibodies against the (foreign and perceived foreign) antigens. B-cells can be activated via T-cell dependent or T-cell independent activation.

The fusion protein of the invention includes a tumor-targeting component, comprising the binding domain of an antibody specifically binding to a tumor antigen. The term “antibody” as used in this invention includes intact molecules as well as functional fragments thereof, such as Fab, F(ab′)2, Fv, scFv, dsFv, or single domain molecules such as VH and VL that are capable of binding to an epitope of an antigen in an MHC restricted manner. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; (6) CDR peptide is a peptide coding for a single complementarity-determining region (CDR); and (7) Single domain antibodies (also called nanobodies), a genetically engineered single monomeric variable antibody domain which selectively binds to a specific antigen. Nanobodies have a molecular weight of only 12-15 kDa, which is much smaller than a common antibody (150-160 kDa).

As a more general statement the term “antibody” aims to encompass any affinity binding entity which binds a cell surface presented molecule with an MHC restricted specificity.

Suitable binding domains of antibody fragments for practicing some embodiments of the invention include a complementarity-determining region (CDR) of an immunoglobulin light chain (referred to herein as “light chain”), a complementarity-determining region of an immunoglobulin alpha (heavy) chain, a variable region of a light chain, a variable region of an alpha chain, a light chain, a heavy chain, an Fd fragment, and antibody fragments comprising essentially whole variable regions of both light and heavy chains such as an Fv, a single chain Fv (scFv), a disulfide-stabilized Fv (dsFv), an Fab, an Fab′, and an F(ab′)2. In specific embodiments, the binding domain of an antibody which specifically binds to said tumor antigen is a single chain Fv (ScFv) or ScFv fragment of the antibody. ScFv fragment is typically a genetically engineered single chain molecule including the variable region of the light chain and the variable region of the heavy chain, linked by a suitable polypeptide linker as a genetically fused single chain molecule.

As used herein, the terms “complementarity-determining region” or “CDR” are used interchangeably to refer to the antigen binding regions found within the variable region of the heavy (e.g. alpha) and light chain polypeptides.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues, which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source, which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

In an embodiment in which the antibody is a full length antibody, the heavy and light chains of an antibody of the invention may be full-length (e.g., an antibody can include at least one, and preferably two, complete heavy chains, and at least one, or two, complete light chains) or may include an antigen-binding portion (a Fab, F(ab′)2, Fv or a single chain Fv fragment (“scFv”)). In other embodiments, the antibody heavy chain constant region is chosen from, e.g., IgG1, IgG2, IgG3, IgG4, IgM, IgA1, IgA2, IgD, and IgE. In some embodiments, the immunoglobulin isotype is selected from IgG1, IgG2, IgG3, and IgG4, more particularly, IgG1 (e.g., human IgG1) or IgG4 (e.g., human IgG4). The choice of antibody type will depend on the immune effector function that the antibody is designed to elicit.

As used herein the term “peptide” refers to native peptides (either proteolysis products or synthetically synthesized peptides) and further to peptidomimetics, such as peptoids and semipeptoids which are peptide analogs, which may have, for example, modifications rendering the peptides more stable while in a body, or more immunogenic. Such modifications include, but are not limited to, cyclization, N terminus modification, C terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modification and residue modification. Methods for preparing peptidomimetic compounds are well known in the art and are specified in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time. According to some embodiments of the invention, but not in all cases necessary, these modifications should exclude anchor amino acids.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the invention may also include one or more modified amino acids or one or more non-amino acid monomers (e.g. fatty acids, complex carbohydrates etc).

As used herein in the specification and in the claims section below the term “amino acid” is understood to include the 20 naturally occurring amino acids; those amino acids often modified post-translationally in vivo, including for example hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids. Further elaboration of the possible amino acids usable according to the present invention and examples of non-natural amino acids useful in the viral-MHC-restricted peptide are given herein under.

The peptides of the invention are preferably utilized in a linear form, although it will be appreciated that in cases where cyclicization does not severely interfere with peptide characteristics, cyclic forms of the peptide can also be utilized.

The viral-MHC-restricted peptides of the invention may include one or more non-natural or natural polar amino acids, including but not limited to serine and threonine, which are capable of increasing peptide solubility due to their hydroxyl-containing side chain.

The peptides of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965. Large scale peptide synthesis is described by Andersson Biopolymers 2000; 55(3):227-50.

Based on accumulated experimental data, it is nowadays possible to predict which of the peptides of a protein will bind to MHC, class I. The HLA-A2 MHC class I has been so far characterized better than other HLA haplotypes, yet predictive and/or sporadic data is available for all other haplotypes.

With respect to HLA-A2 binding peptides, assume the following positions (P1-P9) in a 9-mer peptide:

P1-P2-P3-P4-P5-P6-P7-P8-P9

The P2 and P9 positions include the anchor residues which are the main residues participating in binding to MHC molecules. Amino acid resides engaging positions P2 and P9 are hydrophilic aliphatic non-charged natural amino (examples being Ala, Val, Leu, Ile, Gln, Thr, Ser, Cys, preferably Val and Leu) or of a non-natural hydrophilic aliphatic non-charged amino acid (examples being norleucine (Nle), norvaline (Nva), α-aminobutyric acid). Positions P1 and P3 are also known to include amino acid residues, which participate or assist in binding to MHC molecules, however, these positions can include any amino acids, natural or non-natural. The other positions are engaged by amino acid residues, which typically do not participate in binding, rather these amino acids are presented to the immune cells. Further details relating to the binding of peptides to MHC molecules can be found in Parker, K. C., Bednarek, M. A., Coligan, J. E., Scheme for ranking potential HLA-A2 binding peptides based on independent binding of individual peptide side-chains. J Immunol. 152, 163-175, 1994., see Table V, in particular. Hence, scoring of HLA-A2.1 binding peptides can be performed using the HLA Peptide Binding Predictions software approachable through a worldwide web interface at hypertexttransferprotocol://worldwideweb(dot)bimas(dot)dcrt(dot)nih(dot)gov/molbio/hla_bind/index. This software is based on accumulated data and scores every possible peptide in an analyzed protein for possible binding to MHC HLA-A2.1 according to the contribution of every amino acid in the peptide. Theoretical binding scores represent calculated half-life of the HLA-A2.1-peptide complex.

Hydrophilic aliphatic natural amino acids at P2 and P9 can be substituted by synthetic amino acids, preferably Nleu, Nval and/or α-aminobutyric acid. P9 can be also substituted by aliphatic amino acids of the general formula —HN(CH₂)ₙCOOH, wherein n=3-5, as well as by branched derivatives thereof, such as, but not limited to,

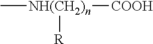

wherein R is, for example, methyl, ethyl or propyl, located at any one or more of the n carbons.

The amino terminal residue (position P1) can be substituted by positively charged aliphatic carboxylic acids, such as, but not limited to, H₂N(CH₂)ₙCOOH, wherein n=2-4 and H₂N—C(NH)—NH(CH₂)ₙCOOH, wherein n=2-3, as well as by hydroxy Lysine, N-methyl Lysine or ornithine (Orn). Additionally, the amino terminal residue can be substituted by enlarged aromatic residues, such as, but not limited to, H₂N—(C₆H₆)—CH₂—COOH, p-aminophenyl alanine, H₂N—F(NH)—NH—(C₆H₆)—CH₂—COOH, p-guanidinophenyl alanine or pyridinoalanine (Pal). These latter residues may form hydrogen bonding with the OH⁻ moieties of the Tyrosine residues at the MHC-1 N-terminal binding pocket, as well as to create, at the same time aromatic-aromatic interactions.

Derivatization of amino acid residues at positions P4-P8, should these residues have a side-chain, such as, OH, SH or NH₂, like Ser, Tyr, Lys, Cys or Orn, can be by alkyl, aryl, alkanoyl or aroyl. In addition, OH groups at these positions may also be derivatized by phosphorylation and/or glycosylation. These derivatizations have been shown in some cases to enhance the binding to the T cell receptor.

Longer derivatives in which the second anchor amino acid is at position P10 may include at P9 most L amino acids. In some cases shorter derivatives are also applicable, in which the C terminal acid serves as the second anchor residue.

Cyclic amino acid derivatives can engage position P4-P8, preferably positions P6 and P7. Cyclization can be obtained through amide bond formation, e.g., by incorporating Glu, Asp, Lys, Orn, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH₂)ₙ—COOH)—C(R)H—COOH or H—N((CH₂)ₙ—COOH)—C(R)H—NH₂, wherein n=1-4, and further wherein R is any natural or non-natural side chain of an amino acid.

Cyclization via formation of S—S bonds through incorporation of two Cys residues is also possible. Additional side-chain to side chain cyclization can be obtained via formation of an interaction bond of the formula —(—CH₂—)ₙ—S—CH₂—C—, wherein n=1 or 2, which is possible, for example, through incorporation of Cys or homoCys and reaction of its free SH group with, e.g., bromoacetylated Lys, Orn, Dab or Dap.

Peptide bonds (—CO—NH—) within the peptide may be substituted by N-methylated bonds (—N(CH₃)—CO—), ester bonds (—C(R)H—C—O—O—C(R)—N—), ketomethylen bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time. Preferably, but not in all cases necessary, these modifications should exclude anchor amino acids.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

As used herein the phrase “tumor antigen” refers to an antigen that is common to specific hyperproliferative disorders such as cancer. Tumor antigens are proteins that are produced by tumor cells that elicit an immune response, particularly T-cell mediated immune responses. The selection of the antigen binding moiety of the invention will depend on the particular type of cancer to be treated.

The type of tumor antigen referred to in the invention includes a tumor-specific antigen (TSA) or a tumor-associated antigen (TAA). A “TSA” is unique to tumor cells and does not occur on other cells in the body. A “TAA” is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

The antigens discussed herein are merely included by way of example. The list is not intended to be exclusive and further examples will be readily apparent to those of skill in the art.

Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

Non-limiting examples of TSA or TAA antigens include the following: Differentiation antigens such as MART-1/MelanA (MART-1), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.291\BCAA, CA 195, CA 242, CA-50, CAM43, CD68\P1, CO—029, FGF-5, G250, Ga733\EpCAM, HTgp-175, M344, MA-50, MG7-Ag, MOV18, NB/70K, NY-CO—1, RCAS1, SDCCAG16, TA-90\Mac-2 binding protein\cyclophilin C-associated protein, TAAL6, TAG72, TLP, and TPS.

In a preferred embodiment, the antigen binding moiety portion of the fusion protein targets an antigen that includes but is not limited to CD19, CD20, CD22, ROR1, Mesothelin, CD33/IL3Ra, c-Met, PSMA, Glycolipid F77, EGFRvIII, GD-2, MY-ESO-1 TCR, MAGE A3 TCR, and the like.

Following are non-limiting sequences of HLA class I-restricted tumor antigens which can bind to the antigen binding domain of the fusion protein of the invention. Tumor antigens suitable for targeting with the fusion protein of the present invention include, but are not limited to the following, non-limiting sequences of HLA class I-restricted tumor antigens which can bind to the antigen binding domain of the antibody which specifically binds to tumor antigens of the invention.

TABLE 3

| | | GenBank Accession No. | SEQ ID NO: of |
|---|---|---|---|
| Cancer | TAA/Marker | of the tumor antigens | the tumor antigens |
| Transitional | Uroplakin II | NP_006751.1 | 113 |
| cell | (UPKII) | | |
| carcinoma | | | |
| Transitional | Uroplakin Ia | NP_001268372.1; | 114; 115 |
| cell | (UPK1A) | NP_0008931.1 | |
| carcinoma | | | |
| Carcinoma | prostate | AAO16090.1 | 116 |
| of the | specific | | |
| prostate | antigen | | |
| | (NPSA) | | |
| Carcinoma | prostate | NP_005663.2 | 117 |
| of the | specific | | |
| prostate | membrane | | |
| | antigen | | |
| | (PSCA) | | |
| Carcinoma | prostate acid | NP_001090.2; | 118; 119; 120 |
| of the | phosphatase | NP_001127666.1; | |
| prostate | (ACPP) | NP_001278966.1 | |
| Breast | BA-46 | NP_001108086.1; | 121; 122 |
| cancer | MFGE8 | NP_005919.2; | |
| | milk fat | | |
| | globule-EGF | | |
| | factor 8 protein | | |
| | [lactadherin] | | |
| Breast | Mucin 1 (MUC1) | NP_001018016.1; | 123; 124; 125; 126; |
| cancer | | NP_001018017.1; | 127; 128; 129; 130; |
| | | NP_001037855.1; | 131; 132; 133; 134; |
| | | NP_001037856.1; | 135; 136; 137; 138; |
| | | NP_001037857.1; | 139; 140; 141; 142 |
| | | NP_001037858.1; | |
| | | NP_001191214.1; | |
| | | NP_001191215.1; | |
| | | NP_001191216.1; | |
| | | NP_001191217.1; | |
| | | NP_001191218.1; | |
| | | NP_001191219.1; | |
| | | NP_001191220.1; | |
| | | NP_001191221.1; | |
| | | NP_001191222.1; | |
| | | NP_001191223.1; | |
| | | NP_001191224.1; | |
| | | NP_001191225.1; | |
| | | NP_001191226.1; | |
| | | NP_002447.4 | |
| Melanoma | premelanosome | NP_001186982.1; | 143; 144; 145 |
| | protein (PMEL; | NP_001186983.1; | |
| | also known as | NP_008859.1 | |
| | Gp100) | | |
| Melanoma | melan-A (MLANA; | NP_005502.1; | 146 |
| | also known as | | |
| | MART1) | | |
| Melanoma, | Melanocortin 1 | | 192 |
| Pancreatic | receptor | | |
| Cancer | (MCR1) | | |
| All | telomerase reverse | NP_001180305.1; | 147; 148 |
| tumors | transcriptase | NP_937983.2 | |
| | (TERT) | | |
| Leukemia | TAX | NP_057864.1; | 149; 150 |
| and | tax p40 | YP_002455788.1 | |
| Burkitts | [Human | | |
| and | T-lymphotropic | | |
| Lymphoma | virus 1] and Tax | | |
| | [Human | | |
| | T-lymphotropic | | |
| | virus 4]; | | |
| Carcinomas | NY-ESO | NP_001318.1 | 151 |
| | cancer/testis | | |
| | antigen | | |
| | 1B | | |
| | (CTAG1B) | | |
| Melanoma | Melanoma | NP_004979.3 | 152 |
| | antigen | | |
| | family A1 | | |
| | (MAGE A1) | | |
| Melanoma | Melanoma antigen | NP_005353.1 | 153 |
| | family A3 | | |
| | (MAGE A3, | | |
| | MAGE-A3) | | |
| Carcinomas | HER2; erb-b2 | NP_001005862.1; | 154; 155; 156; |
| | receptor tyrosine | NP_001276865.1; | 157; 191 |
| | kinase 2 (ERBB2) | NP_001276866.1; | |
| | | NP_001276867.1; | |
| | | NP_004439.2; | |
| Melanoma | Beta-catenine; | NP_001091679.1; | 158; 159; 160 |
| | catenin (cadherin- | NP_001091680.1; | |
| | associated protein), | NP_001895.1; | |
| | beta 1, 88 kDa | | |
| | (CTNNB1) | | |
| Melanoma | Tyrosinase (TYR) | NP_000363.1 | 161 |
| Melanoma | Melanoma-associated | CAA65529.1 | 162 |
| | chondroitin sulfate | | |
| | proteoglycan | | |
| | (MCSP, NGP2, | | |
| | HMWMAA) | | |
| Mesothelioma | Mesothelin | Q13421 | 193 |
| Leukemia | Bcr-abl | AAA35594.1 | 163 |
| Head | caspase8, | NP_001073593.1; | 164; 165; 166; |
| and neck | apoptosis-related | NP_001073594.1; | 167; 168; 169 |
| | cysteine peptidase | NP_001219.2; | |
| | (CASP8) | NP_203519.1; | |
| | | NP_203520.1; | |
| | | NP_203522.1 | |
| Colorectal | Activin A | NP_001097.2 | 170 |
| Cancer | Receptor | | |
| (CRC) | Type 2B | | |
| | (ACVR2B) | | |
| Colorectal | Cadherin EGFLAG | NP_001398.2 | 171 |
| Cancer, | seven-pass G-type | | |
| Stomach | Receptor (CELSR3) | | |
| Cancer | | | |
| Lymphoma | Anaplastic Lymphoma | NP_001340694.1 | 172; 173 |
| (Pediatric) | Kinase (ALK) | NP_004295.2 | |
| Lymphoma | GDNF family | NP_001158510.1 | 174; 175; 176 |
| | Receptor Alpha 2 | NP_001158511.1 | |
| | (GFRA2) | NP_001486.4 | |
| Endometrial, | Delta-like | NP_001304101.1 | 177; 178 |
| Breast | non-canonical | NP_003827.3 | |
| Cancer | Notch ligand 1 | | |
| | (DLK-1) | | |
| Breast, | GDNF | NP_001487.2 | 179 |
| Colorectal, | family | | |
| Endometrial | Receptor | | |
| Cancer | Alpha 3 | | |
| | (GFRA3) | | |
| Endometrial, | G-protein-coupled | NP_061842.1 | 180 |
| Pancreatic | receptor 173 | | |
| Cancer | (GPR173) | | |
| Head and | Insulin receptor- | NP_055030.1 | 181 |
| Neck, | related receptor | | |
| Stomach, | (INSRR) | | |
| Ovarian | | | |
| Cancer | | | |
| Prostate, | Neurotrophic | NP_001007793.1 | 182; 183; 184 |
| Stomach and | tyrosine | NP_001012331.1 | |
| Pancreatic | kinase | NP_002520.2 | |
| Cancer | (NTRK1) | | |
| Colorectal, | Protocadherin- | NP_001290074.1 | 185; 186 |
| Breast, | beta 6 (PCDHB6) | NP_061762.2 | |
| Endometrial | | | |
| Cancer | | | |
| Liver, | Protein | NP_001154912.2 | 187; 188 |
| Stomach, | tyrosine | NP_002833.4 | |
| Colorectal | phosphatase | | |
| and | receptor type H | | |
| Pancreatic | (PTPRH, SAP1) | | |
| Cancer | | | |
| Stomach, | Sidekick | NP_001073121.1 | 189; 190 |
| Thyroid, | Cell Adhesion | NP_689957.3 | |
| Carcinoid | molecule 1 | | |
| Cancer | (SDK1) | | |

The following is a non-limiting list of additional tumor antigens that are expressed on the surface of tumor cells and can be targeted with antibodies (also indicated), suitable for targeting with the antigen-binding domain of the fusion protein of the present invention:

TABLE 4

| | Target | |
|---|---|---|
| mAb | antigen | Indication(s) |
| Alemtuzumab | CD52 | Chronic lymphocytic leukemia |
| Bevacizumab | VEGF | Glioblastoma multiforme, |
| | | colorectal, renal and lung cancer |
| Brentuximab | CD30 | Hodgkin's and anaplastic large cell |
| vedotin | | lymphoma (coupled to MMAE) |
| Catumaxomab | CD3 | Malignant ascites in patients |
| | EPCAM | with EPCAM⁺ cancer |
| Cetuximab | EGFR | HNC and colorectal carcinoma |
| Denosumab | RANKL | Breast cancer, prostate carcinoma |
| | | and giant cell tumors of |
| | | the bone |
| Gemtuzumab | CD33 | Acute myeloid leukemia |
| ozogamicin | | (coupled with calicheamicin) |
| Panitumumab | EGFR | Colorectal carcinoma |
| Pertuzumab | HER2 | Breast carcinoma |
| Ibritumomab | CD20 | Non-Hodgkin lymphoma |
| tiuxetan | | (coupled with ⁹⁰Y or ¹¹¹In) |
| Ofatumumab | CD20 | Chronic lymphocytic leukemia |
| Rituximab | CD20 | Chronic lymphocytic leukemia |
| | | and non-Hodgkin lymphoma |
| Tositumomab | CD20 | Non-Hodgkin lymphoma |
| | | (naked or coupled with ¹³¹I) |

Additional suitable tumor antigens, which are the subject of current clinical trials, include, but are not limited to the following:

TABLE 5

| | Target | | |
|---|---|---|---|
| mAb | antigen | | Indication(s) |
| 1D09C3 | HLA-DR | | CLL |
| | | | Lymphoma |
| AGS-1C4D4 | PSCA | | Pancreatic cancer |
| AVE1642 | IGF1R | | Solid tumors |
| Blinatumomab | CD3 | | Acute lymphoblastic |
| (MEDI-538) | CD19 | | leukemia |
| Carlumab | CCL2 | | Prostate cancer |
| (CNTO 888) | | | Solid tumors |
| Cixutumumab | IGF1R | | Bone or soft- |
| (IMC-A12) | | | tissue sarcomas |
| | | | Renal cell carcinoma |
| Clivatuzumab | MUC1 | | Pancreatic cancer |
| tetraxetan | | | |
| Conatumumab | TRAILR2 | | Colorectal carcinoma |
| (AMG 655) | | | Lung cancer |
| | | | Pancreatic cancer |
| Drozitumab | TRAILR2 | | Colorectal carcinoma |
| (PRO95780) | | | |
| Farletuzumab | FOLR1 | | Ovarian carcinoma |
| (MORAb-003) | | | |
| GC33 | GPC3 | | Hepatocellular carcinoma |
| (RO5137382) | | | |
| Ganitumab | IGF1R | | Breast carcinoma |
| (AMG 479) | | | Pancreatic cancer |
| Inotuzumab | CD22 | | Non-Hodgkin's lymphoma |
| ozogamicin | | | |
| (CMC-544) | | | |
| Intetumumab | ITGA5 | | Prostate cancer |
| (CNTO 95) | | | |
| KRN330 | GPA33 | | Colorectal cancer |
| L19 | FN1 | | Solid tumors |
| Lexatumumab | TRAILR2 | | Solid tumors |
| (HGS-ETR2) | | | |
| Lintuzumab | CD33 | | Acute myeloid leukemia |
| (SGN-33) | | | |
| MIK-β1 (MA1-35896) | IL2RB | | T-LGL leukemia |
| Nimotuzumab | EGFR | | NSCLC |
| (h-R3) | | | |
| Obinutuzumab | CD20 | | Non-Hodgkin's lymphoma |
| (GA101) | | | |
| Rilotumumab | HGF | | Prostrate cancer |
| (AMG 102) | | | |
| Ramucirumab | VEGFR2 | | Hepatocellular carcinoma |
| (IMC-1121B) | | | Gastresophageal |
| | | | adenocarcinoma |
| | | | Lung cancer |
| Trebananib | ANGPT1 | | Solid tumors |
| (AMG 386) | ANGPT2 | | |
| Volociximab | ITGA5 | | NSCLC |
| (M200) | ITGB1 | | |
| | | | Clinical |
| | Target | | Trial |
| mAb | Antigens | Indication(s) | Ref. |
| Alemtuzumab | CD52 | Hematological malignancies | NCT01875237 |
| | | Peripheral T-cell lymphoma | NCT01806337 |
| BC8 | CD45 | Hematological malignancies | NCT01921387 |
| Bevacizumab | VEGF | Brain tumors | NCT01767792 |
| | | Breast carcinoma | NC101894451 |
| | | | NCT01898117 |
| | | | NCT01941407 |
| | | | NCT01959490 |
| | | | NCT01722968 |
| | | Glioma | NCT01891747 |
| | | | NCT01743950 |
| | | Lymphoma | NCT01921790 |
| | | Melanoma | NCT01879306 |
| | | | NCT01950390 |
| | | MM | NCT01859234 |
| | | Ovarian cancer | NCT01735071 |
| | | | NCT01739218 |
| | | | NCT01770301 |
| | | | NCT01838538 |
| | | | NC101847677 |
| | | | NCT01837251 |
| | | | NCT01802749 |
| | | Reproductive | NCT01770171 |
| | | tract cancers | NCT01936974 |
| | | | NCT01821859 |
| | | Rhabdomyosarcoma | NCT01871766 |
| | | Sarcoma | NCT01746238 |
| | | Sarcoma and | NCT01946529 |
| | | neuroectodermal tumors | |
| | | Advanced or metastatic | NCT01831089 |
| | | solid tumors | NCT01749384 |
| | | | NCT01847118 |
| | | | NCT01898130 |
| | | | NC101951482 |
| Blinatumomab | CD3 | DLBCL | NCT01741792 |
| | CD19 | | |
| Brentuximab vedotin | CD30 | AML | NCT01830777 |
| | | DLBCL | NC101925612 |
| | | Germ cell tumors | NCT01851200 |
| | | Lymphoma | NCT01805037 |
| | | | NCT01777152 |
| | | Mast cell leukemia | NCT01807598 |
| | | Peripheral T-cell lymphoma | NCT01841021 |
| Catumaxomab | CD3 | Gastric peritoneal | NCT01784900 |
| | EPCAM | carcinomatosis | |
| | | Ovarian cancer | NCT01815528 |
| Cetuximab | EGFR | Brain tumors | NCT01884740 |
| | | Esophageal cancer | NCT01787006 |
| | | Gastric cancer | NCT01904435 |
| | | Advanced solid tumors | NCT01727869 |
| | | | NC101787500 |
| Ch14.18 | GD2 | Neuroblastoma | NCT01767194 |
| Conatumumab | TRAILR2 | Reproductive | NCT01940172 |
| | | tract cancers | |
| Denosumab | RANKL | NSCLC | NCT01951586 |
| Lintuzumab | CD33 | Leukemia | NCT01756677 |
| Necitumumab | EGFR | NSCLC | NCT01763788 |
| | | | NCT01769391 |
| | | | NCT01788566 |
| Nimotuzumab | EGFR | Breast carcinoma | NCT01939054 |
| | | Cervical cancer | NCT01938105 |
| | | Gastric cancer | NCT01813253 |
| | | NSCLC | NCT01861223 |
| | | Rectal cancer | NCT01899118 |
| Ofatumumab | CD20 | Leukemia | NCT01762202 |
| | | NHL | NCT01768338 |
| Panitumumab | EGFR | Anal cancer | NCT01843452 |
| | | Bladder cancer | NCT01916109 |
| Pertuzumab | HER2 | Gastric cancer | NCT01774786 |
| | | Gastresophageal cancer | |
| Rituximab | CD20 | B-cell malignancies | NCT01905813 |
| | | Hodgkin's lymphoma | NCT01900496 |
| | | Neuroblastoma | NCT01868269 |
| | | Prostate cancer | NCT01804712 |
| SAR650984 | CD38 | MM | NCT01749969 |
| TF2 | CEA | Breast cancer | NCT01730612 |
| | | Medullary thyroid carcinoma | NCT01730638 |
| Trastuzumab | HER2 | Bladder cancer | NCT01828736 |
| | | Recurrent or metastatic | NCT01771458 |
| | | tumors | |

PCT WO 2007 136778 to the instant inventors disclosed a therapeutic engineered antibody-HLA fusion using anti-mesothelin targeting antibody scFv molecule and HLA-A2 loaded with an antigenic epitope, which was able to bind to the surface of mesothelin-expressing tumor cells and render the tumors susceptible to antigen-specific cytotoxic CD8(+) T lymphocytes (CTL)-mediated killing in vitro and in vivo. Thus, according to some embodiments of the invention, the tumor antigen comprises mesothelin.

Mesothelin is a 40 kDa protein that is expressed in the mesothelial cells lining the pleura, peritoneum and pericardium. Although it has been proposed that mesothelin may be involved in cell adhesion, its biological function remains unclear. Mesothelin is immunogenic.

Mesothelin is over expressed in several human tumors, including mesothelioma and ovarian and pancreatic adenocarcinoma. The interaction between mesothelin and MUC16 (also known as CA125) may facilitate the implantation and peritoneal spread of tumors by cell adhesion. The region (296-359) consisting of 64 amino acids at the N-terminal of cell surface mesothelin is the functional binding domain for MUC1. In some specific embodiments, the MCSP tumor antigen has an amino acid sequence comprised in SEQ ID NO: 193.

In some embodiments, the tumor antigen comprises the melanoma-associated chondroitin sulfate proteoglycan (CSPG4, MCSP) or neuron-glial 2 (NG2) antigen. MCSP, also known as high-molecular weight melanoma-associated antigen (HMW MAA). MCSP is expressed on the majority (>90%) of human melanoma tissues and melanoma cell lines but not on carcinoma, fibroblastoid cells, or cells of hematological origin. MCSP is also highly expressed on the surface of dysplastic nevi. In specific embodiments, the MCSP tumor antigen is human MCSP (Accession nos. CAA65529, AAQ62842.1 or NP 001888). In some specific embodiments, the MCSP tumor antigen has an amino acid sequence comprised in SEQ ID NO: 162.

An additional model for hematological malignancies is the CD25 receptor. In some embodiments, the CD25 tumor antigen has an amino acid sequence comprised in SEQ ID NO: 194.

It will be appreciated that the fusion protein of the present invention or portions thereof can be prepared in several ways, including solid phase protein synthesis. However, in specific embodiments of the invention, at least major portions of the molecules, e.g., the alpha chain of a human MHC class I molecule, the viral MHC-restricted peptide, the beta-2-microglobulin, linkers, the binding domain of an antibody which binds to a tumor antigen, etc. are generated by translation of a respective nucleic acid construct or constructs encoding the molecule. Exemplary methods for preparation of fusion proteins suitable for preparation of the fusion proteins of the present invention are detailed in PCT Application WO2007/011953 to the present inventors.

According to an aspect of some embodiments of the invention there is provided an isolated polynucleotide comprising a nucleic acid sequence encoding the fusion protein, or component polypeptide sequences thereof of some embodiments of the invention.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

The term “isolated” refers to at least partially separated from the natural environment e.g., from a cell, or from a tissue, e.g., from a human body.

The isolated polynucleotide can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

According to an aspect of some embodiments of the invention there is provided a nucleic acid construct comprising an isolated polynucleotide comprising a nucleic acid sequence encoding the molecule of some embodiments of the invention and a cis-acting regulatory element for directing transcription of the isolated polynucleotide in a host cell.

Thus, the expression of natural or synthetic nucleic acids encoding the fusion protein of the invention is typically achieved by operably linking a nucleic acid encoding the fusion protein or portions thereof to a cis-acting regulatory element (e.g., a promoter sequence), and incorporating the construct into an expression vector.

The nucleic acid construct of the invention may also include an enhancer, a transcription and translation initiation sequence, transcription and translation terminator and a polyadenylation signal, a 5′ LTR, a tRNA binding site, a packaging signal, an origin of second-strand DNA synthesis, and a 3′ LTR or a portion thereof; additional polynucleotide sequences that allow, for example, the translation of several proteins from a single mRNA such as an internal ribosome entry site (IRES) and sequences for genomic integration of the promoter-chimeric polypeptide; sequences engineered to enhance stability, production, purification, yield or toxicity of the expressed peptide.

Enhancers regulate the frequency of transcriptional initiation. Typically, promoter elements are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1.alpha. (EF-1.alpha.). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence, which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The isolated polynucleotide of the invention can be cloned into a number of types of vectors. For example, the isolated polynucleotide can be cloned into a vector including, but not limited to a plasmid, a phagemid, a phage derivative, an animal virus, and a cosmid. Vectors of particular interest include expression vectors, replication vectors, probe generation vectors, and sequencing vectors.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Currently preferred in vivo or in vitro nucleic acid transfer techniques include transfection with viral or non-viral constructs, such as adenovirus, lentivirus, Herpes simplex I virus, or adeno-associated virus (AAV). Recombinant viral vectors offer advantages such as lateral infection and targeting specificity. Introduction of nucleic acids by viral infection offers several advantages over other methods such as lipofection and electroporation, since higher transfection efficiency can be obtained due to the infectious nature of viruses.

Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

According to some embodiments of the invention, the nucleic acid construct of the invention is a viral vector.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

The nucleic acid construct of some embodiments of the invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

In order to assess the expression of a fusion protein or portions thereof, the nucleic acid construct to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Various methods can be used to introduce the nucleic acid construct of the invention into a host cell, e.g., mammalian, bacterial, yeast, or insect cell. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, physical, chemical, or biological means (e.g., stable or transient transfection, lipofection, electroporation and infection with recombinant viral vectors). In addition, see U.S. Pat. Nos. 5,464,764 and 5,487,992 for positive-negative selection methods.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

In the case where a non-viral delivery system is utilized, an exemplary delivery vehicle is a liposome.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances, which may be naturally occurring or synthetic lipids. For example, lipids include the fatty droplets that naturally occur in the cytoplasm as well as the class of compounds, which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”); and other lipids may be obtained from Avanti Polar Lipids, Inc, (Birmingham, Ala.). Additionally or alternatively, the DOTMA, DOPE, and DC-Chol [Tonkinson et al., Cancer Investigation, 14(1): 54-65 (1996)] lipids can be used. Stock solutions of lipids in chloroform or chloroform/methanol can be stored at about −20 degrees C. Chloroform is used as the only solvent since it is more readily evaporated than methanol.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Thus, according to an aspect of some embodiments of the invention there is provided an isolated cell comprising the polynucleotide of some embodiments of the invention or the nucleic acid construct of some embodiments of the invention.

It will be appreciated that a fusion protein of the invention whose amino acid sequence includes the N-terminal amino acid methionine, likely represents the fusion protein as expressed in a bacterial cell. Depending on the specific bacterial cell employed to express the fusion protein, the N-terminal methionine may be cleaved and removed. Accordingly, it is contemplated that fusion proteins in accordance with this invention encompass both those with, and those without, an N-terminal methionine. In general, when a fusion protein in accordance with the invention is expressed in a eukaryotic cell, it would lack the N-terminal methionine. Therefore, it is to be appreciated that the amino acid sequence of expressed fusion proteins according to the invention may include or not include such N-terminal methionine depending on the type of cells in which the proteins are expressed.

Whenever and wherever used, the linker peptide(s) is selected of an amino acid sequence which is inherently flexible, such that the polypeptides connected thereby independently and natively fold following expression thereof, thus facilitating the formation of a functional fusion protein comprising active viral-MHC restricted peptide, active human beta-2-microglobulin-alpha chain of human MHC class I molecule, active antibody binding domain of an anti-tumor antigen antibody complex.

Whenever co-expression of independent polypeptides in a single cell is of choice, the construct or constructs employed must be configured such that the levels of expression of the independent polypeptides are optimized, so as to obtain highest proportions of the final product.

Yeast cells can also be utilized as host cells by the present invention. Numerous examples of yeast expression vectors suitable for expression of the nucleic acid sequences of the present invention in yeast are known in the art and are commercially available. Such vectors are usually introduced in a yeast host cell via chemical or electroporation transformation methods well known in the art. Commercially available systems include, for example, the pYES™ (Invitrogen™ Corporation, Carlsbad Calif., USA) or the YEX™ (Clontech® Laboratories, Mountain View, Calif. USA) expression systems.

It will be appreciated that when expressed in eukaryotic expression systems such as those described above, the nucleic acid construct preferably includes a signal peptide encoding sequence such that the polypeptides produced from the nucleic acid sequences are directed via the attached signal peptide into secretion pathways. For example, in mammalian, insect and yeast host cells, the expressed polypeptides can be secreted to the growth medium, while in plant expression systems the polypeptides can be secreted into the apoplast, or directed into a subcellular organelle.

The present inventors have shown that targeting of tumor cells with fusion proteins comprising a tumor antigen binding domain and MHC class I molecules allogeneic (e.g. mismatched) to the recipient can effectively inhibit, and even reverse tumor development, eliciting site-specific allogeneic T-cell recruitment through an MHC-restricted peptide. Thus, the fusion protein, and compositions of matter comprising the fusion protein can be used for treatment of tumors in individuals in need thereof.

Thus, according to an aspect of some embodiments of the invention there is provided a pharmaceutical composition comprising the fusion protein or composition of matter of some embodiments of the invention and a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the fusion protein or composition of matter of some embodiments of the invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the invention thus may be formulated in conventional manner using one or more physiologically acceptable carriers comprising excipients and auxiliaries, which facilitate processing of the active ingredients into preparations, which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

The administration of the pharmaceutical composition may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the pharmaceutical composition of the present invention is administered to a patient by intradermal or subcutaneous injection. In another embodiment, the pharmaceutical composition of the present invention is preferably administered by i.v. injection. The pharmaceutical composition may be injected directly into a tumor, lymph node, or site of infection.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the active ingredients to allow for the preparation of highly concentrated solutions.

Alternatively, the active ingredient may be in powder form for constitution with a suitable vehicle, e.g., sterile, pyrogen-free water based solution, before use. The pharmaceutical composition of the invention may also be formulated in rectal compositions such as suppositories or retention enemas, using, e.g., conventional suppository bases such as cocoa butter or other glycerides.

Pharmaceutical compositions suitable for use in context of the invention include compositions wherein the active ingredients are contained in an amount effective to achieve the intended purpose. More specifically, a therapeutically effective amount means an amount of active ingredients effective to kill tumor cells, prevent, alleviate or ameliorate symptoms of a tumor-related pathology or prolong the survival of the subject being treated.

Determination of a therapeutically effective amount is well within the capability of those skilled in the art, especially in light of the detailed disclosure provided herein.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). Fusion protein or composition of matter may also be administered multiple times at these dosages. The optimal dosage and treatment regime for a particular patient can readily be determined by one skilled in the art of medicine by monitoring the patient for signs of disease and adjusting the treatment accordingly.

For example, the effect of the active ingredients (e.g., the fusion protein or composition of matter of some embodiments of the invention on the tumor-related pathology can be evaluated by monitoring the level of markers, e.g., cytokines, hormones, glucose, peptides, carbohydrates, etc. in a biological sample of the treated subject using well known methods.

Data obtained from in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to some embodiments of the invention, the therapeutic agent of the invention can be provided to the subject in conjunction with other drug(s) designed for treating the pathology [combination therapy, (e.g., before, simultaneously or following)].

In certain embodiments of the present invention, the compositions of matter or allogenic fusion proteins described herein are administered to a patient in conjunction with any number of relevant treatment modalities, including but not limited to chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In a further embodiment, the compositions of matter or allogenic fusion proteins of the present invention are administered to a patient in conjunction with (e.g., before, simultaneously or following) bone marrow transplantation or before or following surgery, for example, tumor resection.

The combination therapy may increase the therapeutic effect of the agent of the invention in the treated subject, and may increase the therapeutic effect of the other treatment modalities.

Compositions of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

In some embodiments, there is provided a method of killing a tumor cell presenting a tumor antigen, comprising administering to an individual a composition-of-matter comprising at least one fusion protein of the invention, wherein the alpha chain of a human MHC molecule is allogeneic to the individual, so as to elicit an alloimmune response to the tumor cell presenting the antigen, thereby killing the tumor cell.

As used herein, the terms “subject”, “patient” or “individual” includes mammals, preferably human beings at any age which suffer from the tumor.

The tumor can be, but is not limited to a cancerous tumor.

The term “cancer” as used herein is defined as disease characterized by the rapid and uncontrolled growth of aberrant cells. Cancer cells can spread locally or through the bloodstream and lymphatic system to other parts of the body.

The cancer may be a hematological malignancy, a solid tumor, a primary or a metatastizing tumor. Examples of various cancerous tumors include but are not limited to, breast cancer tumors, prostate cancer tumors, ovarian cancer tumors, cervical cancer tumors, skin cancer tumors, pancreatic cancer tumors, colorectal cancer tumors, renal cancer tumors, liver cancer tumors, brain cancer tumors, lymphoma, Chronic Lymphocytic Leukemia (CLL), leukemia, lung cancer tumors and the like. Additional non-limiting examples of cancerous tumors, which can be treated by the method of some embodiments of the invention, are provided in Tables 3, 4 and 5 above.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the fusion proteins or composition of matter of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

According to some embodiments of the invention, the tumor is a solid tumor.

According to some embodiments of the invention, administration of the fusion protein or composition of matter of some of the embodiments of the invention has an anti-tumor effect, killing tumor cells. The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the tumor. An “anti-tumor effect” can also be manifested by the ability of the fusion protein or composition of matter of the invention to prevent the occurrence of tumor in the first place.

Allogenicity (e.g. MHC mismatch) is determined relative to the MHC class-1 type of the individual (e.g. recipient, patient). Thus, in some embodiments, the MHC class I type of the individual (e.g. patient) is determined prior to administering of the composition of matter of the present invention. Methods for determining the MHC type of individuals (Human Leukocyte Antigen Oligotyping; Sequence-based Typing, Histocompatibility Testing) are well known in the art, and include typing from a blood or other tissue sample (e.g. buccal swab) of the individual, and HLA screen of the individual's sample. The HLA screen can include an HLA antibody screen using lymphocytotoxicity testing, which tests the function of the individual's (e.g. patient's) lymphocytes when presented with a panel of HLA-specific antibodies and complement, as well as molecular techniques (e.g. PCR) for determining the sequence of the individuals' HLA genes (and, subsequently, the amino acid sequence of the individual's (e.g. patient's) HLA polypeptide.

The present invention also envisions multiple, repeated administration of the composition of matter comprising the fusion protein to the same individual, in a plurality of successive cycles of administration (further detailed below), in general, in order to overcome diminished allogeneic rejection response and/or production of host anti-fusion protein antibodies. According to some embodiments, where successive cycles of administration comprise administering fusion proteins having human MHC class I alpha chains mismatched to those of the individual (e.g. patient) and non-identical to those of the previously administered compositions of matter, a minimal number of three (or more) different allo-molecule treatment cycles for each patient. In specific embodiments, the combinations of human MHC alpha chain allotypes are selected based on the clustering of the HLA-A, HLA-B and HLA-C alleles in order to generate as few as seven versions based on HLA A (see, for example, FIG. 15A) and six versions based on HLA B (see, for example, FIGS. 15B and 15C). In further embodiments, following selection of specific target populations, fewer than seven versions of the HLA-A alleles and fewer than six versions of the HLA-B alleles can suffice for successive cycles of administration comprise administering fusion proteins having human MHC class I alpha chains mismatched to those of the individual (e.g. patient) and non-identical to those of the previously administered compositions of matter. In still further embodiments, and since certain genotype combinations are less represented in the population, as few as four versions of the human MHC class I alpha chains can suffice for successive cycles of administration comprise administering fusion proteins having human MHC class I alpha chains mismatched to those of the individual (e.g. patient) and non-identical to those of the previously administered compositions of matter. In specific embodiments, the alloimmune response of the individual's (e.g. patient's) tumor cells to the administration of the composition of matter or fusion protein of the invention is assessed (at least one week) following administration, and a new cycle of administration of the composition of matter or fusion protein of the invention is commenced upon detection of reduced alloimmune response to the alpha heavy chain of the human MHC class I allogeneic molecule.

Pre-determined combinations of non-identical, allogeneic fusion proteins can be useful for treatment with the compositions of matter, fusion proteins and methods of the present invention, particularly when a plurality of cycles of administration is envisaged. Thus, in some embodiments, there is provided a composition-of-matter comprising a plurality of fusion proteins, each fusion protein comprising a viral MHC-restricted peptide; a human beta-2-microglobulin; an alpha chain of a human MHC class I molecule and a binding domain of an antibody which specifically binds to a tumor antigen, wherein the plurality of fusion proteins comprises at least two non-identical fusion proteins having different allogeneic human MHC class I molecule alpha chains. In some embodiments, the plurality of fusion proteins comprises at least 2, 3, 4, 5, 6, 7, 8, 9, 10 or more non-identical fusion proteins having different allogeneic human MHC class I molecule alpha chains. In other embodiments the different allogeneic human MHC class I molecule alpha chains are selected from the human MHC class I molecule alpha chains described in detail herein.

The present invention also envisages an article of manufacture comprising a plurality of fusion proteins each packaged in a different package, each fusion protein comprising a viral MHC-restricted peptide; a human beta-2-microglobulin; an alpha chain of a human MHC class I molecule and a binding domain of an antibody which specifically binds to a tumor antigen, wherein said plurality of fusion proteins comprises at least two non-identical fusion proteins having different allogeneic human MHC class I molecule alpha chains.

In specific embodiments, the composition of matter or article of manufacture of the present invention comprises an alpha chain of the non-identical human MHC class I molecules selected from the group consisting of HLA-A23, HLA-A32, HLA-A74, HLA-A31, HLA-A80, HLA-A36, HLA-A25, HLA-A26, HLA-A43, HLA-A34, HLA-A66, HLA-A69, HLA-A68, HLA-A29, HLA-B14, HLA-B18, HLA-B27, HLA-B38, HLA-B39, HLA-B41, HLA-B42, HLA-B47, HLA-B48, HLA-B49, HLA-B50, HLA-B52, HLA-B53, HLA-B54, HLA-B55, HLA-B56, HLA-B57, HLA-B58, HLA-B59, HLA-B67, HLA-B73, HLA-B78, HLA-B82, HLA-B81. In other specific embodiments, the alpha chain of the non-identical human MHC class I molecule has an amino acid sequence at least 95% identical to an amino acid sequence selected from the group consisting of HLA-A23:01:01 (SEQ ID NO: 44), HLA-A32:01:01 (SEQ ID NO: 47), HLA-A74:01:01 (SEQ ID NO: 55), HLA-A31:01:02 (SEQ ID NO: 57), HLA-A80:01:01 (SEQ ID NO: 49), HLA-A36:01 (SEQ ID NO: 56), HLA-A25:01:01 (SEQ ID NO: 45), HLA-A26:01:01(SEQ ID NO: 52), HLA-A43:01(SEQ ID NO: 53), HLA-A34:01:01(SEQ ID NO: 48), HLA-A66:01:01(SEQ ID NO: 50), HLA-A69:01:01(SEQ ID NO: 51), HLA-A68:01:01(SEQ ID NO: 54), HLA-A29:01:01(SEQ ID NO: 46), HLA-B14:01:01(SEQ ID NO: 58), HLA-B18:01:01(SEQ ID NO: 59), HLA-B27:02:01(SEQ ID NO: 60), HLA-B38:01:01(SEQ ID NO: 61), HLA-B39:01:01(SEQ ID NO: 62), HLA-B41:01:01(SEQ ID NO: 63), HLA-B42:01:01(SEQ ID NO: 64), HLA-B47:01:01(SEQ ID NO: 65), HLA-B48:01:01(SEQ ID NO: 66), HLA-B49:01:01(SEQ ID NO: 67), HLA-B50:01:01(SEQ ID NO: 68), HLA-B52:01:01(SEQ ID NO: 69), HLA-B53:01:01(SEQ ID NO: 70), HLA-B54:01:01(SEQ ID NO: 71), HLA-B55:01:01(SEQ ID NO: 72), HLA-B56:01:01(SEQ ID NO: 73), HLA-B57:01:01(SEQ ID NO: 74), HLA-B58:01:01(SEQ ID NO: 75), HLA-B59:01:01(SEQ ID NO: 76), HLA-B67:01:01(SEQ ID NO: 77), HLA-B73:01(SEQ ID NO: 78), HLA-B78:01:01(SEQ ID NO: 79), HLA-B82:01(SEQ ID NO: 80), HLA-B81:01 (SEQ ID NO: 81).

In some embodiments, predetermined combinations of fusion proteins with different viral MHC-restricted peptides can be useful. Thus, in some embodiments there is provided a composition-of-matter comprising a plurality of fusion proteins, each fusion protein comprising a viral MHC-restricted peptide; a human beta-2-microglobulin; an alpha chain of a human MHC class I molecule and a binding domain of an antibody which specifically binds to a tumor antigen, wherein the plurality of fusion proteins comprises at least two non-identical fusion proteins having different viral MHC-restricted peptides. In some embodiments, the plurality of fusion proteins comprises at least 2, 3, 4, 5, 6, 7, 8, 9, 10 or more non-identical fusion proteins having different viral MHC-restricted peptides. Exemplary MHC-restricted peptides suited for use with the fusion proteins and composition of matter of the present invention include, but are not limited to the following list of viral MHC-restricted peptides:

TABLE 6

| SEQ | | | |
|---|---|---|---|
| ID | | HLA | Amino |
| NO | Organism, Protein | Restriction | Acids |
| 84 | Zaire ebolavirus, | A*23:01, | AYQGD |
| | Nucleoprotein, NP(82-90) | A*24:02 | YKLF |
| | Used in HLA-A23 | | |
| | Dextamers | | |
| 85 | Dengue virus, Genome | A*23:01, | RFLEFE |
| | polyprotein GP(2973-2982) | A*24:02 | ALGF |
| 86 | Vaccinia virus, Primase | A*23:01, | VWINNS |
| | D5(349-357) | A*24:02, | WKF |
| | | A*29:02, | |
| | | A*30:02 | |
| 87 | Vaccinia virus, | A*23:01 | TYNDHI |
| | Thymidylate Kinase(58-66) | | VNL |
| 88 | Human Herpesvirus 5 | A*23:01, | AYAQKI |
| | (hCMV5), Immediate- | A*24:02 | FKIL |
| | early Protein 1(248-257) | | |
| | Used in HLA-A24 | | |
| | Tetramers | | |
| 89 | Human Herpesvirus 4 | A*23:01, | PYLFWL |
| | (Epstein Ban virus, | A*24:02, | AAI |
| | EBV), Latent membrane | A*24:03, | |
| | protein 2(131-139) | A*30:02, | |
| | Used in HLA-A23 | A*02:01 | |
| | Tetramers | | |
| 90 | Yellow fever virus 1D7, | A*23:01, | IYGIFQS |
| | Genome polyprotein | A*24:02 | TF |
| | (1508-1516) Used in | | |
| | HLA-A24 Tetramers | | |
| 91 | H. sapiens, Insulin | A*24:02 | LWMRL |
| | Protein PPI(3-11) Used | | LPLL |
| | in HLA-A24 Tetramers | | |
| 92 | Influenza A, | A*24:02, | FYIQMC |
| | Nucleoprotein NP(39-47) | A*23:01, | TEL |
| | | A*29:02 | |
| 93 | P. falciparum 37D | A*24:02, | SFLFVE |
| | (Malaria), | A*23:01, | ALF |
| | circumsporazoite protein | A*29:02 | |
| | CSP(12-20) | | |
| 94 | P. falciparum 37D | A*23:01 | VFNVVN |
| | (Malaria), | | SSI |
| | circumsporazoite protein | | |
| | CSP(377-385) | | |
| 95 | H. sapiens, Elongation | A*66:01, | YFDPAN |
| | factor 2(265-273) | A*24:02, | GKF |
| | | A*23:01, | |
| | | A*30:01, | |
| | | A*01:01, | |
| | | B*35:01, | |
| | | B*15:16 | |
| 96 | Influenza A, Polymerase | A*24:02, | YYLEKA |
| | acidity protein(130-138) | A*23:01, | NKI |
| | | A*24:03 | |
| 97 | Influenza A, Polymerase | A*24:02, | FYRYGF |
| | subunit (496-505). HLA- | A*23:01, | VANF |
| | A*24:02 structure is | A*29:02 | |
| | available | | |
| | (E. coli→Refolding→X- | | |
| | ray) | | |
| 98 | H. sapiens, Cyclin- | A*24:02, | KYFDEH |
| | dependent Kinase | A*30:04, | YEY |
| | Regulatory | A*23:01, | |
| | Subunit 2(11-19) | A*29:02, | |
| | *Mostly cellular MS data | B*35:01 | |
| 99 | Dengue virus 2, Genome | A*23:01, | IQKETL |
| | polyprotein GP(512-520) | A*24:02, | VTF |
| | | B*15:01 | |
| 100 | Dengue virus 2, Genome | A*23:01, | IQMSSG |
| | polyprotein GP(550-559) | | NLLF |
| 101 | Dengue virus 2, Genome | A*23:01, | SYSMCT |
| | polyprotein GP(578-586) | | GKF |
| 102 | H. sapiens, ( ) | A*23:01, | AYVPGF |
| | | | AHI |
| 103 | H. sapiens, ( ) | A*23:01, | KYLSVQ |
| | | | GQF |
| 104 | H. sapiens, ( ) | A*23:01, | KYQEVT |
| | | | NNL |
| 105 | H. sapiens, ( ) | A*23:01, | LYDPVIS |
| | | | KL |
| 106 | H. sapiens, ( ) | A*23:01, | RYIANT |
| | | | VEL |
| 107 | H. sapiens, ( ) | A*23:01, | RYLEQL |
| | | | HQL |

The present invention also envisages an article of manufacture comprising a plurality of fusion proteins each packaged in a different package, each fusion protein comprising a viral MHC-restricted peptide; a human beta-2-microglobulin; an alpha chain of a human MHC class I molecule and a binding domain of an antibody which specifically binds to a tumor antigen, the plurality of fusion proteins comprising at least two non-identical fusion proteins having different viral MHC-restricted peptides.

In some specific embodiments, the viral MHC-restricted peptides are 8 or 9 amino acids in length.

The present invention also envisages pluralities of fusion proteins targeted to different, non-identical tumor antigens. Such combinations of non-identical tumor antigens can be useful, for example, for repeated cycles of administration as well as targeting multiple sites on tumor cell, or tumors comprising cells expressing diverse but characteristic tumor antigens. Thus, in some embodiments, there is provided a composition-of-matter comprising a plurality of fusion proteins of the invention wherein the plurality of fusion proteins comprises at least two non-identical fusion proteins having a different binding domain of an antibody, which specifically binds to a tumor antigen. In specific embodiments, the plurality of fusion proteins comprises at least 2, 3, 4, 5, 6, 7, 8, 9, 10 or more non-identical fusion proteins having different binding domain of an antibody, which specifically binds to a tumor antigen. In some embodiments, the different binding domains can be of antibodies that target the same tumor antigen, while in other embodiments the different binding domains can be of antibodies that target and specifically bind to distinct and separate tumor antigens. In some embodiments, the tumor antigens can be different antigens of the same tumor peptide/polypeptide.

The present invention also envisages an article of manufacture comprising a plurality of fusion proteins each packaged in a different package, each fusion protein comprising a viral MHC-restricted peptide; a human beta-2-microglobulin; an alpha chain of a human MHC class I molecule and a binding domain of an antibody which specifically binds to a tumor antigen, wherein the plurality of fusion proteins comprises at least two non-identical fusion proteins having a different binding domain of an antibody which specifically binds to a tumor antigen.

In specific embodiments, the binding domain of an antibody, which specifically binds to a tumor antigen, selected from the non-limiting list of tumor antigens described in Tables 3, 4 and 5. In some embodiments, the tumor antigen is mesothelin. In further embodiments, the tumor antigen is MCSP. In still further embodiments, the tumor antigen is the CD25 receptor.

The present invention also envisages a “bank” of polynucleotides for production of any of the articles of manufacture, compositions or fusion proteins of the invention, in order to provide rapid and even automated access to sequences encoding effective combinations of fusion proteins of the invention. Thus, in some embodiments, there is provided an expression system comprising a plurality of nucleic acid vectors each encoding a different human MHC class I alpha chain, wherein the plurality of nucleic acid vectors comprises vectors encoding at least two non-identical human MHC class I alpha chains. In some embodiments, the plurality of nucleic acid vectors comprises at least 2, 3, 4, 5, 6, 7, 8, 9, 10 or more vectors encoding non-identical human MHC class I alpha chains having different human MHC class I molecule alpha chains. In other embodiments, the vectors encode different allogeneic human MHC class I molecule alpha chains selected from the human MHC class I molecule alpha chains described in detail herein.

The present invention also envisages similar expression systems comprising pluralities of nucleic acid vectors, each encoding a different viral MHC restricted peptide, or each encoding a different binding domain of an antibody, which specifically binds to a tumor antigen. Combinations between the nucleic acid vectors of the expression systems described herein, as well as nucleic acid sequences or vectors encoding the linkers and beta2-microglobulin of the invention could provide nucleic acid vectors, or pluralities of nucleic acid vectors encoding the fusion proteins, or component sequences of the fusion proteins, articles of manufacture or compositions of the present invention

Employment of a specific MHC-restricted peptide is advantageous since it avoids use of anti-CD3, which causes global T cell recruitment and cytokine syndrome. In some embodiments, the MHC-restricted peptide is a viral-derived (e.g. influenza-derived) peptide.

Using a fusion protein comprising a viral MHC restricted-peptide provides the opportunity to vaccinate the recipient (individual, patient) against influenza (or the specific flu peptide) prior to the treatment with the fusion protein. This combined approach can increase the number of precursor memory effector T cells that are recruited to the tumor site via the antibody-MHC fusion molecules. Thus, in some embodiments, the individual (e.g. patient) is vaccinated against the virus of the viral MHC restricted peptide prior to the treatment with the composition of matter or fusion protein as described here.

The optimal degree of sequence difference between a given patient's genotype and the allo-HLA of the treatment molecule is an important consideration for the development of the targeted allogeneic approach, in order to establish the correlations between the genotype the blood donor and the sequences of allo-molecules, so that a decision-tree for identifying the most effective fusion proteins and mismatched alpha MHC class I molecule(s) for each patient can be proposed. An ex-vivo experimental system that allows the testing of the ability of different allo-HLA molecules to initiate CTL dependent allo-rejection of autologous target cells is thus an important aspect of treatment in the targeted allogeneic approach.

Thus, in some embodiments of the present invention there is provided an assay for identifying allogeneic human MHC class I alpha chains effective for eliciting an alloimmune response in a subject, the assay comprising:

i) contacting peripheral blood mononuclear cells (PBMC)-derived T cells from the subject with antigen presenting cells from a donor mismatched for MHC class I, thereby activating the T cells;
ii) isolating and culturing the T cells;
iii) contacting the T-cells with
a) a CD19+ B-cell target cell of the subject, and
b) a fusion protein comprising a viral MHC-restricted peptide; a human beta-2-microglobulin; an alpha chain of a human MHC class I molecule HLA-mismatched for the subject and a binding domain of an antibody which specifically binds CD19, and
iv) assaying an immune response of the B-cells,
v) repeating steps i)-iv) using an autologous fusion protein comprising the viral MHC-restricted peptide; the human beta-2-microglobulin and an alpha chain of a human MHC class I molecule HLA-matched for the subject, and
vi) determining effectiveness of the allogeneic human MHC class I alpha chain for eliciting an alloimmune response in the subject by comparing the immune response of said B-cells of the allogeneic with that of the autologous fusion protein.

In some embodiments, the immune response of the B cells is selected from the group consisting of direct killing of the B-cells, cytokine secretion and T cell activation markers. B-cell cytokines suitable for measurement in the assay include, but are not limited to IL-2, IL-4, TNFα, IL-6 (Be-2 cells), IFNγ, IL-12 and TNFα. Direct killing of the cells can be assessed by any currently available assays, for example, vital staining, cellular impedance (e.g. xCELLigence, ACEA Biosciences), ⁵¹Cr release. LDH-release, etc. T-cell activation assays are well known in the art, for example, proliferation assays, cytokine assays, and the like. “Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

In some embodiments, the “target cell” of the assay can be another cell of the subject, which displays a specific antigen—in such a case, the binding domain of the fusion protein will be a binding domain of an antibody which specifically binds that antigen, and the measure of target cell killing can be designed to suit the specific character of the target cell.

Determining the effectiveness of the allogeneic human MHC class I alpha chain for eliciting an alloimmune response in the subject can be effected, in some embodiments, by measuring the relative intensities of the target cell (e.g. B-cell) immune response using mismatched and autologous fusion proteins. For example, in some embodiments, an alloimmune response 10%, 15%, 20%, 25%, 30%, 40%, 50%, 60%, 70%, 80%, 100%, 150%, 200%, 300%, 400% or more greater than the response elicited with an autologous fusion protein is considered effective. In other embodiments, the character of the target cells response (e.g. direct cell killing, cytokine secretion, T cell markers) can be used as an indication of the effectiveness of the elicited response—for example, elicitation of direct cell killing and cytokine secretion of the target cell with an allogeneic fusion protein compared with only cytokine secretion using an autologous fusion protein can indicate elicitation of an effective response with the allogeneic fusion protein. In some embodiments, effectiveness is determined by evaluation of both the intensity and the character of the elicited response.

Performing these experiments on PBMCs from donors with different degrees of sequence identity compared to the therapeutic allo-molecule can enable elucidation of optimal correlations between the sequence diversity and the optimal allogeneic T cell functional parameters measured.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

The following section provides specific examples for each of the various aspects of the invention described herein. These examples should not be regarded as limiting in any way, as the invention can be practiced in similar, yet somewhat different ways. These examples, however, teach one of ordinary skills in the art how to practice various alternatives and embodiments of the invention.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### EXPERIMENTAL PROCEDURES

Plasmids, DNA and Protein Sequences

Mammalian expression plasmid, pCDNA3.1, and all DNA cassettes for expression in the Expi293 system were ordered from GeneArt (Invitrogen). Human MCSP Expression: pEF6-CSPG4-Myc-His (Addgene Plasmid #69037). Mouse B2-microglobulin (NCBI Gene ID: b2m: 12010), H-2Kb, H-2Kd, H-2Kk and H-2Kq (MGI, alleles of H-2K, ID: 95894) and anti-MCSP clone 225.28S light and heavy variable chains (Kabat: Light ID: 029888, Heavy ID: 029889) protein sequences were taken from the listed online data bases.

Hybridoma B Cell Lines and Antibody Production

Murine hybridoma cell lines were cultured in complete RPMI (CRPMI) according to ATCC recommendations, for antibody secretion the HB-79 cells were transferred to serum free CRPMI supplemented with Biogro-2 SFM and TIB-139 were cultured in antibody depleted CRPMI in CELLine Classic 350 ml flasks. The antibodies were purified from the growth media using columns loaded with protein A or G sepharose beads (Millipore, A for mouse anti H-2Kd (34-1-2S) IgG2a and G for mouse anti H-2Kb (B8-24-3) IgG1) washed with 5 column volumes (CV) of sodium hydro-phosphate (Na₂HPO₄0.02M pH 8) binding buffer. Fractions were eluted with citrate buffer (pH3) and immediately adjusted to pH 7 by 1M Trizma base (pH 8). The antibody containing fractions were unified and transferred to PBS by over-night dialysis.

Expi293 Transients PEI Transfection and Protein Preparation

Expi293 cells were cultured in PETG filter capped flasks (Nalgene) with serum-free Expi293 media (Gibco) at a 37° C., 8% CO₂ 120-125 rpm shaker humidifier incubator and passaged according to manufacture recommendations. One day prior to transfection, 98-100% viable cells were seeded at 2 million cells per milliliter of cell culture media; 30 or 300 milliliters depending on the flask size, 125 ml or 1000 ml flask respectively, and the required amount of protein to be produced, in the range of 0.5-1 mg or 3-6 mg, respectively. On the next day, filter—sterilized pCDNA plasmid coding for the desired protein molecule (1 μg of DNA per milliliter of the final cell culture media volume, 30 μg or 300 μg) was mixed in to 1/10 volume of desired culture media volume (3 ml or 30 ml for desired final culture media volume of 30 ml and 300 ml respectively), then filter-sterilized polyethylenimine (PEI) (25K PEI, 2 μg/ml, pH7, Polysciences) was added at 3:1 mass ratio (PEI:DNA), vortexed and the transfection mix incubated for 15-20 minutes at RT. Expi293 cells were re-seeded at ˜1.1 million cells/ml in 9/10 of the desired culture media volume (27 ml or 270 ml for desired final culture media volumes of 30 ml and 300 ml respectively). The transfection mix was then added to the cells to make a final volume of 30 or 300 milliliters, the cells transferred to an incubator and grown for 6-7 days, then the medium was separated from cells and debris by centrifugation (3000 g, 20 min, 4° C.) and passed through a 0.22 micron filter. For purification via His Tag, 1 ml packed volume of TALON metal affinity resin (Takara-Clontech) for every ˜20 ml of harvested expression media was washed thrice with 0.1% PBST wash buffer (PBS, 0.1% Tween, pH 8). The washed resin, mixed with the expression media, was incubated at RT with slow rotation for ˜45 minutes, and then loaded onto a disposable column (Bio-Rad). The Resin was washed with 10 times the volume of the resin, [“Column Volume” (CV)], of wash buffer, 2.5 times CV of 1 mM and 2.5 times CV of 5 mM Imidazole (Sigma) in wash buffer. Protein fractions of ˜200 μl were eluted by adding 100 mM Imidazole. Protein concentration was estimated using Coomassie Plus Bradford Assay Kit (Pierce) and Fractions containing the TALON bound protein were combined. Salts and Tween were removed by overnight dialysis against PBS at 4° C. Coomassie staining of gels following SDS-PAGE electrophoresis was performed following each purification, to verify that the correctly sized protein was produced and that the enrichment procedure via His Tag affinity column was satisfactory.

Small Scale Expression and Western Blot

Small scale transfection of Expi293 cells (2 ml in a 6 well plate) was performed for each plasmid to check protein expression and binding to TALON resin. The expression media was prepared as described above, 1 ml was incubated with washed 50 μl TALON at room temperature (RT) with slow rotation for ˜45 minutes. The beads were separated from the media by centrifugation (350 g, 5 minutes) and washed thrice with 1 ml of 0.1% PBST. 50 μl of protein sample buffer ×2 (Bio-Rad) was added and samples were heated to 95° C. for 10 minutes. The TALON-precipitated samples and the input (harvested media) samples were loaded onto home-made SDS 12% poly-acrylamide gels along with Precision Plus protein size marker (Bio-Rad). After running, protein transfer to a nitrocellulose membrane (Whatman) was performed by wet-transfer (200 mAmp, 1-2 hours, 4° C.). The membranes were blocked with 5% non-fat milk in PBS (5% MPBS) for 30 minutes and then the ladders were separated from the membrane to prevent binding of the primary antibody to the His-Tagged standard proteins. The membrane was incubated with 10 ml of mouse anti His-Tag IgG1 (clone AD1.1.10, Bio-Rad) diluted 1/1000 in 5% MPBS, over night at 4° C. with rotation. The next day, the membrane was washed four times with 0.1% Tween, 2 mM Tris and 15 mM NaCl pH 7.4 (0.1% TBST). Secondary HRP conjugated goat anti mouse antibody (Jackson Immuno-Research) was diluted 1/1000 in 5% MPBS, incubated with the membrane for 30 minutes at RT with shaking and washed thrice with 0.1% TBST. WesternBright ECL reagent (Advansta) was used to assay HRP activity and the luminescence signal imaged using the ImageQuant LAS 4000 instrument (GE Healthcare Life Sciences).

BirA Biotinylation

To biotinylate proteins for sandwich ELISA, making tetramers or staining cells for flow cytometry, the BirA Biotin-protein Ligase Bulk Reaction Kit (Avidity) was used. 0.5 ml of 0.3-0.5 mg/ml protein with the BirA tag (GLNDIFEAQKIEWH, SEQ ID NO: 31) in the carboxy [C] terminus of the protein sequence was transferred to Tris buffer (10 mM Tris Hydrochloride pH 8.1) by overnight dialysis at 4° C. The protein was mixed with 62 μl of Biomix A, 620 of Biomix B, 100 biotin and 1.25 μl BirA enzyme, the biotinylating reaction was incubated at 30° C. for 3 hours or overnight at 25° C. Biotin removal and buffer change was done by overnight dialysis to PBS at 4° C.

H-2Kb/d Sandwich ELISA

Wells of a 96 well Nunc MaxiSorp plates (Thermo Scientific) were coated with 100 μl of 1 μg/ml Biotinylated BSA (Sigma) in PBS, overnight at 4° C. Next, the wells were washed (thrice with 2000 PBS) and coated with 100 μl of 10 μg/ml Streptavidin (Promega) in PBS for 30 minutes at RT. The wells were washed and coated with 30-50 μl of the indicated concentration (0-10 μg/ml) of biotinylated complex or peptide-MHC-scFv molecule in PBS for 1 hour at RT. The plates were washed and blocked with 100 μl 2% Milk in PBS (2% MPBS) for 30 minutes. After washing with PBS, the wells were incubated with 50-100 μl mouse antibody diluted in 2% MPBS (mouse serum diluted 1/1000, 10 μg/ml anti-His Tag clone AD1.1.10, Bio-Rad, 10 μg/ml anti-H-2Kb or H-2Kd purified from B cell hybridoma supernatant) for 1 hour at RT. Wells were washed with PBS and incubated for 1 hour with 100 μl of 1/1000 anti-mouse HRP (Jackson Immuno-Research) in 2% MPBS. Wells were washed and incubated with one volume (60-100 μl) of TMB reagent (SouthernBiotech) at RT in the dark for 0.5-2 minutes. The reaction was stopped by adding ½ volume of stop solution (2N H2504) and the absorbance at 450 nm and 420 nm was measured using Epoch Instrument (BioTek). The signal was calculated by: (450-420 nm)_{Sample}−(450-420 nm)_{Background}, for every well, average and standard error was calculated for each sample from triplicate wells and analyzed by ANOVA test.

B16F10 Culture, Transfection and Isolation of Stable Cell Lines

Adherent B16F10 murine melanoma cells were cultured in 10 cm plates with 10% FCS, 10 mg/ml HEPES, Glutamine and Pen-Strep supplemented DMEM and maintained at up to 80% confluency. The cells were typically passaged every two days by washing with PBS and incubating with 1 ml of EDTA (Invitrogen) Trypsin (Difco) in PBS at 37° C. for 1 minute and then 9 ml of fresh pre-warmed media was added and the cells passaged 1/20 and seeded in new 10 cm plates. One day before transfection, the cells were seeded at 25-45% confluence, the next day the confluence was 50-80% and transfection was performed using x-fect reagent (Clonetech). Plasmid DNA (pEF-6 Blast) coding for human MCSP (AddGene) and reagent complex was prepared in un-supplemented DMEM, as recommended by the manufacturer. The cells were transfected in a drop-wise manner and after 24-48 hours Blasticidin-S (InvivoGen) was added at a concentration of 4 μg/ml to select for transfected cells. The cells were passaged every two days 1/20 for two weeks. To isolate stably transfected clones, the cells were seeded at a highly diluted concentration of ˜5-6 cells/ml and plated at 150 ul/well in 96 well plates without selection and grown for five days, in order to isolate clones originating from single cells. The isolated clones were collected and re-plated in 24 well plates with selection and surviving clones were tested for MCSP expression by staining and flow cytometry. The positive clones were expanded and aliquots stored in liquid nitrogen. At the same time one plate of each MCSP-expressing clone was re-seeded in a selection-free medium and passaged for 3 weeks to test the stability of MCSP expression without selection. Two clones that had consistent MCSP expression levels, C8 and C25, were expanded and tested in-vivo.

Tetramer Preparation

To make fluorophore conjugated peptide-MHC tetramers, a 50 μl aliquot of ˜0.3 mg/ml biotinylated peptide-MHC complex was thawed on ice. An appropriate amount (˜1:1 molar ratio) of APC conjugated Streptavidin (Jackson Immuno-Research) was sequentially added, 1/10 of the final amount each time, at 10 minutes intervals on ice and in the dark.

Splenocyte Isolation

Spleens were harvested from euthanized mice (C57BL6 or BalbC) and put into a wash buffer (PBS 2% FCS). A single cell suspension was prepared by gently disrupting the spleen against a 100 micron nylon mesh with the back-end of a syringe plunger. The mesh was washed with PBS 2% FCS and the cells pelleted by centrifugation at 360 g for 10 minutes at 4° C. The pelleted cells were resuspended in 1-3 ml of Red Blood Cell lysis buffer (Sigma) and incubated at RT for 3-5 minutes. 30 ml of PBS with 2% FCS and 1 mM EDTA (MACS buffer) were added and the cells centrifuged again at 360×g for 10 minutes. The pelleted splenocytes were resuspended with 3 ml of MACS and live cells counted using a hemocytometer and Trypan blue (Sigma) staining.

Tumor Single Cell Suspension Preparation

B16F10 Tumors were excised from euthanized tumor-bearing C57BL6 mice, cut into small 5 mm diameter pieces and transferred to PBS 2% FCS at 4° C. The pieces were pelleted by gravity for 3 minutes and the supernatant replaced with 3 ml of RPMI supplemented with 2% FCS, 0.5 mg/ml Collagenase D (Roche) and 100 μg/ml DNase I (Sigma). The digestion mix was incubated at 37° C. for 35-45 minutes with sequential pipetting to break the tumor into increasingly smaller pieces. Then, 2 ml of RPMI supplemented with 2% FCS, 1 mg/ml Collagenase/Dispase (Roche) and 100 μg/ml DNase I (Sigma) was added and incubated for additional 10 to 15 minutes, until a satisfactory single-cell suspension was achieved. 0.5 M EDTA pH 8 (Invitrogen) was added, stopping Dispase activity, to a final concentration of 2 mM. The cells were passed through a 40 micron nylon mesh, pelleted and washed once with MACS buffer by centrifugation at 700 g for 3 minutes at 4° C.

Staining for Flow Cytometry

The splenocyte or tumor single cell suspension was diluted, ˜10⁷ cells/ml or 5M cells/ml respectively, with MACS and incubated on ice for 30 minutes with 1 μl/well Fc blocker (Biolegend). For CD8-PE and Tetramer-APC staining, 1 million cells (100 μl) were mixed with 5 μl APC conjugated tetramer (1.25 μg biotinylated peptide-MHC complex per 1 million cells) in U shaped 96 well plates and incubated on ice and in the dark for 1 hour. Then 10/well of PE conjugated anti-mouse CD8 (Biolegend) was added, mixed and incubated for another 30 minutes. For T cell phenotype analysis, 1 million cells (100 μl) were pelleted by centrifugation, 700 g for 3 minutes at 4° C., and stained for 1 hour by resuspension in 100 μl MACS with FITC conjugated anti-CD8, APC-Cy7 conjugated anti-CD4, PE conjugated anti-CD44 and APC conjugated anti-CD62L (Biolegend) at 1:100 dilution. Before analyzing by LSR-2 (BD), the cells were washed thrice by centrifugation, 300 g for 3 minutes at 4° C., and resuspension in fresh 1500 MACS buffer.

Subcutaneous Melanoma and Treatment

Low passaged B16F10 (WT cells) and MCSP expressing B16 melanoma (Clone C25 cells) were passaged 1/20 three days before the injection to mice. Two to three days before injection, 7-8 week old C57BL6 female mice were shaved on the right-lower back. On the day of injection, B16F10 cells were collected by Trypsinization as described and washed four times with PBS by centrifugation, 700 g for 3 minutes at 25° C. The cells were suspended as 1M or 10M cells/ml with PBS, for the WT and C25 cells respectively. Using a 1 ml syringe with a 25G needle, 100 μl of mixed cell suspension was subcutaneously injected to the lower back of the mice. For the following days the mice were followed, every 2-3 days the mice were weighed and tumor length (L) and width (W) were measured by caliper. On day 6-7, the tumor volume (calculated: ½*W²*L) was 25-50 mm³ and the mice received a daily tail vein injection of 200 μl PBS or 0.5 mg/ml protein (100 μg) in PBS as indicated, for 5 consecutive days. The mice were sacrificed on day 15-17, at which point some of the experimental groups had tumors of 1.5 cm diameter or more.

Mouse Serum Collection

Upon euthanizing treated and mock treated mice, blood was collected by heart puncture using a 1 ml syringe with a 21G needle and transferred to Eppendorf tubes with 25 μl Heparin (5K Units/ml, LEO). The serum was separated from the RBCs and PBMCs by centrifugation, 1000 g at RT for 20 minutes, and slowly pipetting the clear top fraction. The serum was passed through 0.22 micron filter and kept frozen at −20° C.

Mouse Serum ELISA and Competition ELISA

Most steps were performed as described for sandwich ELISA, but instead of incubating with anti-His or anti-H-2Kb/d fold sensitive antibodies, the wells were incubated with mouse serum diluted in 2% MPBS 1:1000. For competition ELISA, final concentration of 100 ug/ml un-biotinylated complex (CG-11) in PBS was added to the 1:1000 diluted serum and incubated for 30 minutes before it was added to the wells as indicated in the relevant figure.

### Example 1

### Design of Soluble Murine Single Chain Peptide-MHC Complexes and Peptide-MHC Anti-MCSP scFV Fusion Protein

As a model system for antibody-mediated targeting of allogeneic MHC, the human Melanoma-associated Chondroitin Sulfate Proteoglycan (MCSP) antigen was selected. MCSP is expressed on the cell surface in 80% of Human Melanomas. MCSP has 84% sequence identity to the mouse homologue. A published sequence of an MCSP specific scFv is available (see Neri et al, 1996)(SEQ ID NO: 27) and was shown to be very specific and with high affinity (225.28S, Ka=4.8×10⁸ M⁻¹). MCSP can be transfected into a mouse melanoma cell line—B16-F10, which can be used to produce several types of cancer models in C57BL/6 mice.

To design the soluble single chain MHC that precedes the ScFv, the 2QRI structure in the Protein Data Base (PDB) was used. As shown in the middle panel (M15) of FIG. 2 the peptide is followed by a 15 amino acid linker of (G₄S)₃, β-2-microglobulin (SEQ ID NO: 20), a linker (G4S)₃ (SEQ ID NO: 16) or (G4S)₄ (SEQ ID NO: 18), the H-2a subunit (H2Kb, SEQ ID NO: 22), a short connector (linker) of 4 amino acids (ASGG), the ScFv of 225.28S (SEQ ID NO: 27) and finally a His tag (SEQ ID NO: 29) for purification. A molecule with a BirA Tag (BirA Tag-SEQ ID NO: 31) (FIG. 2 top panel “BA”) and a soluble complex with a BirA Tag, but without the ScFv, that could be used for MHC tetramer staining of T cells (FIG. 2 bottom panel, “CG”) were also designed. Since the object was to induce allogeneic rejection of B16-F10 tumors in C57BL/6 mice that express the H-2Kb allele, the sequence of this allele was used as a negative control molecule that would not cause allogeneic rejection. The MHC mRNA sequences for the allogeneic rejection alleles were derived from the GenBank database (H-2Kk-U47330.1(SEQ ID NO: 195), H-2Kd-U47329.1(SEQ ID NO: 196), H-2Kq-BC080812.1(SEQ ID NO: 197)), compared to the H2Kb (SEQ ID NO: 22) 2QRI protein structure sequence to identify the corresponding part of the sequence to be used in the allogeneic H-2Kd molecule (SEQ ID NO: 24). Table 7 below lists the similarity and identity of the three alleles that were considered, the H-2Kk, H-2Kd and H-2Kq with low, middle and high degree of differences respectively.

TABLE 7

| MHC I | Mouse | Score | Similarity | % Similarity | Identity | % identity | Gaps | Difference |
|---|---|---|---|---|---|---|---|---|
| H-2Kk | C3H/He | 521 | 260/280 | 92 | 251/280 | 90 | 0 | Low |
| H-2Kd | Bulb/C | 481 | 252/280 | 90 | 231/280 | 83 | 0 | Middle |
| H-2Kq | FVB/N | 451 | 238/280 | 85 | 214/280 | 76 | 0 | High |

The H-2 domain differences between H-2Kb and other alleles based on protein sequences.

Due to the overall high similarity between all H-2 alleles, it was suggested that using alleles with a greater difference would cause a response that is less peptide-specific. Thus, the frequency of T cell clones that recognize the H-2 molecule, regardless of the peptide bound to it will be high. Therefore, there may be an optimal degree of difference, in which the allogeneic T cell response is not too strong (causing a cytokine storm), but not too weak that it fails to reject the tumor. As a first stage, the present inventors focused on the H-2Kd complex bound to three different influenza derived peptides (SEQ ID NOs. 9, 10 and 11). As mentioned, for the negative control the H-2Kb was used with a murine peptide; YAMI peptide (SEQ ID NO: 12) of the Mdm2 protein that is frequently over expressed in tumors, RTYT peptide (SEQ ID NO: 13) of the Catenin β-1 protein and SGYD (SEQ ID NO: 14) of the sterol regulatory element-binding protein. The peptides and their SYFPEITHI binding scores are listed in the following table:

| | | | Peptide-MHC | | |
|---|---|---|---|---|---|
| | | | affinity | | |
| | Peptide/SEQ | MHC | SYFPEITHI | | |
| # | ID NO. | Class I | score | Organism | Protein |
| 1 | LYQNVGTYV/ | H-2Kd | 29 | Influenza A | Hemagglutinin |
| | SEQ ID NO 9 | | | | |
| 2 | IYSTVASSL/ | H-2Kd | 30 | Influenza A | Hemagglutinin |
| | SEQ ID NO: 10 | | | | |
| 3 | TYQRTRALV/ | H-2Kd | 24 | Influenza A | Nucleoprotein |
| | SEQ ID NO: 11 | | | | |
| 4 | YAMIYRNL/ | H-2Kb | 23 | Mus | Mdm2 |
| | SEQ ID NO: 12 | | | Musculus | |
| 5 | RTYTYEKL/ | H-2Kb | 29 | Mus | Catenin β-1 |
| | SEQ ID NO: 13 | | | Musculus | |
| 7 | SGYDFSRL/ | H-2Kb | 30 | Mus | Sterol regulatory |
| | SEQ ID NO: 14 | | | Musculus | element-binding |
| | | | | | protein |

A mammalian expression system, Expi293F HEK cells, was used which is compatible and safe for producing proteins for in-vivo use. As mentioned, the peptide was covalently linked to the MHC, allowing for it to be folded together with the MHC inside the cells and then be secreted into the growth media. As the expression vector, the pcDNA3.1 plasmid was used which has a strong CMV promoter. A mammalian secretion signal sequence was added for secretion (SEQ ID NO: 2, encoded by SEQ ID NO: 1). To assist in cloning, a cassette system that allows the generation of all the DNA sequence combinations needed was used (FIG. 2) in a simple cut-paste-transform process using the Golden Gate enzyme—AarI. Each of the 7 cassettes, [4 peptides (SEQ ID NOs.: 3, 4, 5 and 6), 1 beta2m (SEQ ID NO: 19), 2 MHC I (H-2Kb, SEQ ID NO: 21 and H-2Kd, SEQ ID NO: 23) and 1ScFv (SEQ ID NO: 26)], was optimized for expression in Expi293 cells and purchased from Gene-Art. Individual combinations were designated according to the generic backbone (“CG”, “BA” or “M15”), a number indicating the peptide used (1, 2, 3, 4, 5, 6 or 7, according to Table 8) and additional numbers indicating the individual clones, for example: “CG1 . . . ” fusion proteins are CG backbone with LYQNVGTYV peptide, while “CG3 . . . ” fusion proteins are CG backbone with TYQRTRALV peptide, “M151 . . . ” fusion proteins are M15 backbone with LYQNVGTYV peptide, etc.

### Example 2

### Soluble Murine Single Chain Peptide-MHC Complexes and Peptide-MHC Anti-MCSP scFV Fusion Protein is Successfully Expressed in the Expi293 System

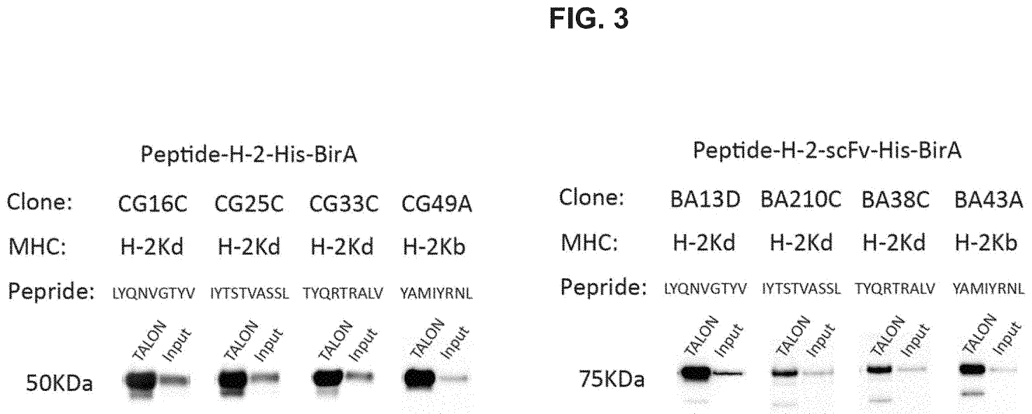

Verification of the expression of the Ab-MHC fusions in Expi293 was performed in small scale: 2 ml culture was transfected with PEI reagent, the supernatant collected after one week, precipitated using TALON beads, washed, run on a gel and western blotted using an anti-His antibody. The expected sizes were about 75 KDa for the full sized molecule (peptide-MHC-I-ScFv-His/His-BirA tagged, “BA”) and about 50KDa for the peptide-MHC-I-His-BirA tagged (“CG”). All variants were successfully expressed and resulted in correctly sized bands (FIG. 3).

Representative fusion proteins expressed in the Expi293 system included CG soluble fusion protein: H2Kb molecule with YAMIYRNL peptide with Tags, without the scFv (SEQ ID NO: 33), encoded by SEQ ID NO: 32; BA soluble fusion protein: H2Kb molecule with YAMIYRNL peptide anti MCSP scFv of 225.28S clone and tags (SEQ ID NO: 35), encoded by SEQ ID NO: 34; M15 soluble fusion protein: H2Kb molecule with YAMIYRNL peptide anti MCSP scFv of 225.28S clone and tags (SEQ ID NO: 37), encoded by SEQ ID NO: 36; CG Soluble fusion protein: H2Kd molecule with TYQRTRALV peptide with Tags, without the scFv (SEQ ID NO: 39), encoded by SEQ ID NO: 38; BA Soluble fusion protein: H2Kd molecule with TYQRTRALV peptide anti MCSP scFv of 225.28S clone and tags (SEQ ID NO: 41), encoded by SEQ ID NO: 40; and M15 Soluble fusion protein: H2Kd molecule with TYQRTRALV peptide anti MCSP scFv of 225.28S clone and tags (SEQ ID NO: 43), encoded by SEQ ID NO: 42.

### Example 3

### Optimizing the G4S Linker Connecting the β2-Microglobulin (β2M) to the H-2K α Chain Improved Complex Yield and Stability

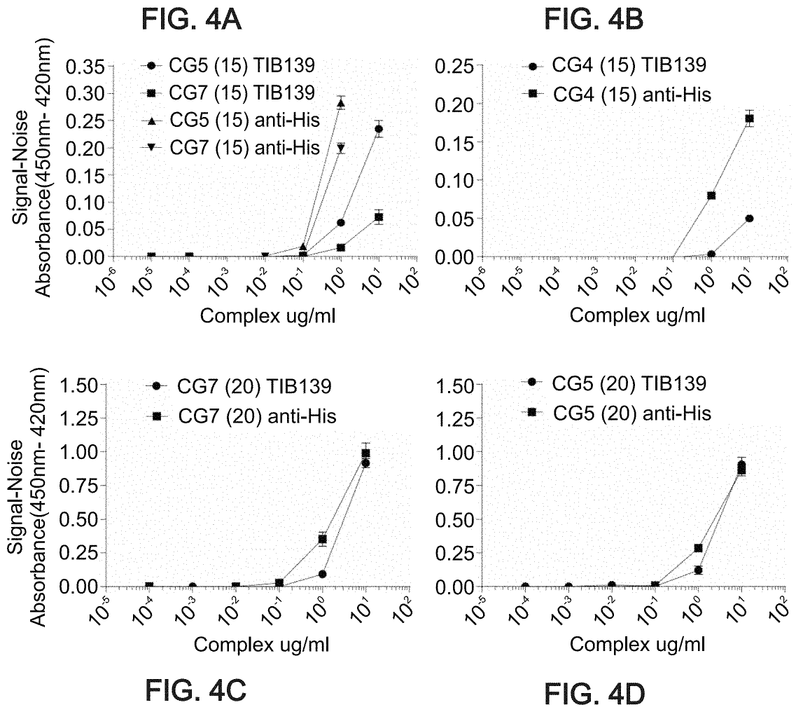

Next, the effector arm of the fusions, i.e. the MHC-peptide moiety, was tested. The molecules were expressed in a medium-scale, 30 ml Expi293 culture, crudely purifying it by binding to TALON beads, washing the column with up to 5 mM Imidazole and eluting fractions with 100 mM. After dialysis, the BirA tagged molecules were biotinylated with a BirA enzyme. The murine B cell Hybridoma cell lines; HB79 and TIB139 that produce antibody clones 34-1-2S (IgG2a) and B8-24-3 (IgG1), recognizing the folded forms of H-2Kd and H-2Kb, respectively were used. These antibodies were employed in ELISA assays: each complex was biotinylated and used to coat wells via PBS-biotin and Streptavidin. By this method the signal of 34-1-2S or B8-24-3 fold specific antibodies can be compared to the anti-His tag (clone AD1.1.10 from Bio-Rad), non fold specific antibody, thus allowing comparison between complexes with different peptides and linker lengths. As seen in FIGS. 4A and 4B, the RTYT and SGYD complexes are more stable than the YAMI peptide linked H-2Kb complex with 15 amino acid long β-2-microglobulin—MHC linker. In addition, comparison of the plots revealed that the 20 amino acid long β-2-microglobulin—MHC linker (FIGS. 4C and 4D) is superior in folding compared with the 15 amino acid long linker (FIGS. 4A and 4B). The linker-length effect observed in this assay was present in all the H-2Kb molecules. The H-2Kd molecules showed stable folding with both linker lengths (data not shown).

Importantly, the protein yield of soluble complex or fusion molecule with a 20 amino acid long β-2-microglobulin—MHC linker was almost double in efficiency compared to the 15 amino acid long. This effect was observed for both the H-2Kb and the H-2Kd molecules.

### Example 4

### Peptide-MHC Fusion Molecules Binds MCSP Via the Specific scFv Derived from the 225.28s Antibody

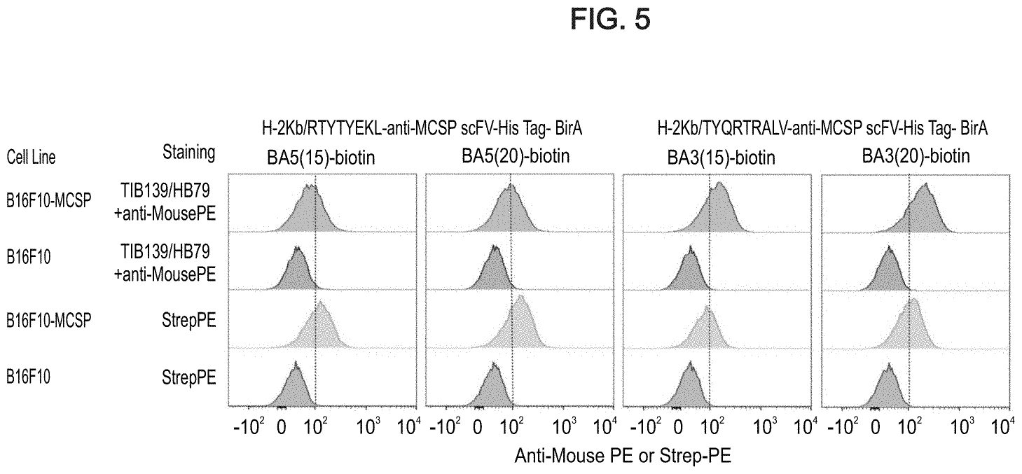

The next objective was to test the correct folding of the anti-MCSP scFv portion of the molecules. B16F10 cells of clone C25 (expressing MCSP) and WT control B16F10 cells were stained with the biotinylated ScFv-MHC molecules, washed and incubated with PE conjugated streptavidin (Strep-PE) or a fold-specific anti-H-2K mouse antibody (34-1-2S or B8-24-3) and then subjected to another step of anti-mouse PE staining. As shown in FIG. 5, analysis by flow cytometry showed that the molecules could bind the MCSP expressing C25 (B16F10-MCSP), but not the B16F10 WT cells (B16F10), indicating that the ScFv was folded correctly and functional. Here also, the 20 amino acid long β-2-microglobulin—MHC linker was superior in staining intensity compared with the 15 amino acid long linker (representative flow cytometry in FIG. 5 shows one representative H-2Kb allele peptide and one representative H-2Kd allele peptide).

### Example 5

### Naïve CD8 Positive Splenocytes Bind Allogeneic Single Chain Peptide-MHC Tetramers

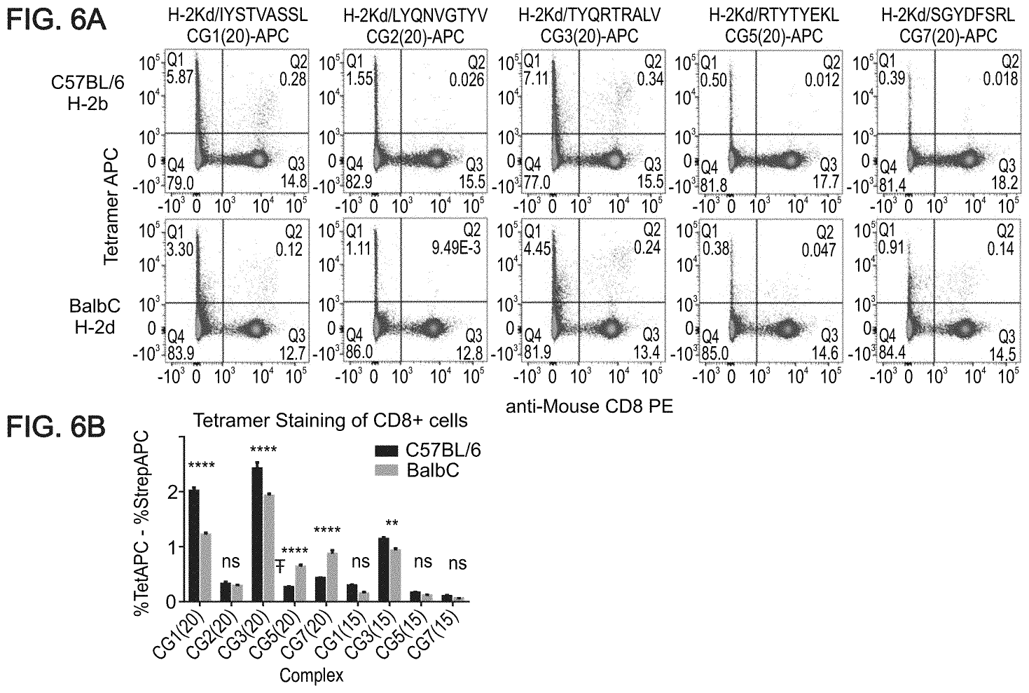

Due to the high dissociation rate of MHC monomers, detection of T-cell-MHC binding is performed using MHC tetramers, which can bind multiple MHCs to a T-cells, increasing binding avidity. To test whether the soluble single chain peptide-MHC molecules are capable of being recognized by CTLs, tetramers were prepared by gradually adding APC conjugated streptavidin to different biotinylated complexes. Splenocytes purified from naïve C57BL/6 (H-2b) and BalbC (H-2d) mice, contacted with tetramers for 1.5 hours and then phycoerythrin (PE)-conjugated anti-CD8 antibody added for the last 30 min of incubation. Dot plots in FIG. 6A show representative staining data from two mice. For several tetramers the percent of tetramer positive CD8 expressing allogeneic cells is higher than the syngeneic cells (H-2Kd tetramers staining of C57BL/6 is higher than H-2Kb, and the opposite for BalbC cells). The histogram in FIG. 6B summarizes the percentages of tetramer positive CD8+ cells of the different tetramers with 15 amino acid or 20 amino acid long β-2-microglobulin—MHC linker, from a single staining experiment. A significantly higher percentage of allogeneic vs syngeneic tetramer binding CD8 cells was observed with the 20 amino acid, but not the 15 amino acid long linker. Staining the influenza H-2Kd CG1 and CG3 tetramers generally resulted in higher percentages of tetramer positive CD8 cells than the self-peptide H-2Kb CG5 and CG7 complexes.

### Example 6

### Human MCSP Expressing B16F10 Murine Melanoma Cells Form Subcutaneous Tumors when Injected to C57BL/6 Naïve Mice

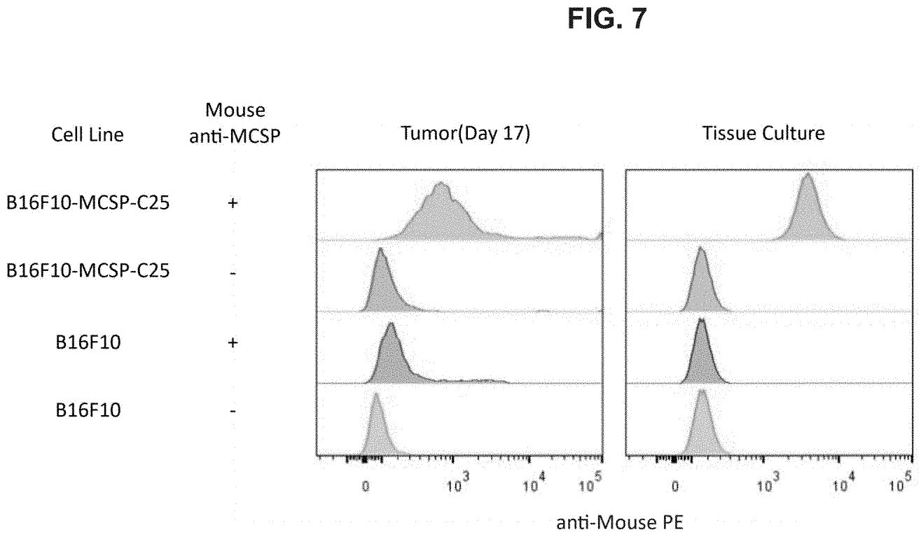

To generate an MCSP-expressing B16F10 melanoma cell line, the MCSP coding DNA in a mammalian expression vector (pEF) from the Add Gene depository was used and transfected into B16F10 cells. After two weeks of Blasticidin selection, the surviving cells were diluted and single cells seeded in 96 well plates. Screening for MCSP-expressing clones was performed by flow cytometry, using an anti-MCSP monoclonal mouse antibody and phycoerythrin (PE) conjugated anti-mouse antibody. MCSP-expressing clones were expanded in selection media and frozen. Cell plates were maintained without selection for 3 weeks and MCSP expression was analyzed again by flow cytometry, and two clones (C8 and C25) expressed MCSP at high levels. Both clones had the same growth rate in tissue culture as the original B16F10, but the C25 clone had a slightly higher expression level than the C8 clone (data not shown). To test if these clones were capable of producing tumors in mice, C57BL/6 mice were subcutaneously injected with 100 μl of different concentration of C8, C25 or B16F10 WT cells and the diameters of the tumors measured every 3 days for two weeks. Some of the C8 tumors did not grow, while all the C25 and B16F10 WT produced tumors. The growth rate of the C25 tumors was slower than the original B16F10 cell line, injecting one million cells of C25 produced tumors that were similar in size to 1/10 of a million B16F10 WT cells (data no shown). To confirm that the C25 clone does not lose MCSP expression in vivo, single-cell suspensions were prepared from excised tumors, MCSP stained and analysed by flow cytometry. Single cell suspensions was prepared by digesting for about 40 minutes with a mixture of Collagenase, Dispase and DNase I. After staining of the cells for MCSP the results, shown in FIG. 7, indicated that all the C25 tumor cells express MCSP in-vivo. However, the MCSP staining intensity of C25 tumors cells was lower than that of the C25 cell line cells that were collected from tissue culture plates, where one-minute incubation with Trypsin and EDTA was used to make a single cell suspension. Without wishing to be bound by a particular theory, the MCSP staining intensity difference may the result of proteolytic activity of the protease Dispase used in the tumor single cell purification protocol. However, when Collagenase was used without Dispase, the melanoma cells were not properly detached from each other and the result was not satisfactory, making it difficult to assess the effect of Dispase. The ex-vivo staining assay was repeated on more than 3 separate occasions, with more than 5 C25 tumors and at least 2 B16F10 WT tumors each time, with similar results, thus confirming that the C25 B16F10 clone does not lose MCSP expression in-vivo.

### Example 7

### Tumor Infiltrating Lymphocytes (TIL) are Present in MCSP Positive B16F10 Tumors and the Frequencies of Memory and Effector CD4 and CD8 Populations are Similar to Those Found in the WT B16F10 Tumors

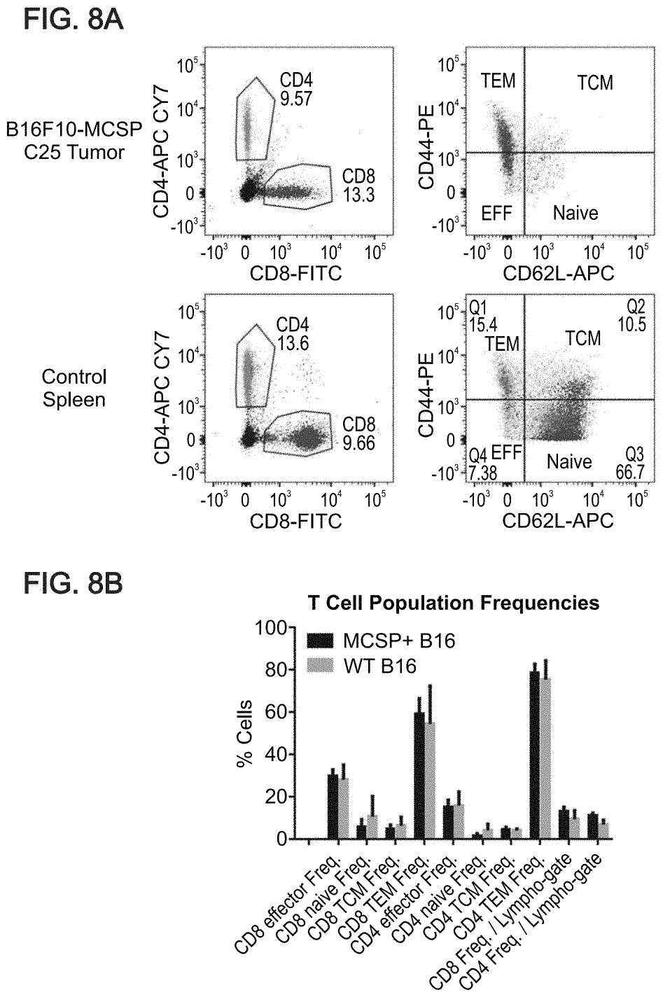

Next the present inventors confirmed that MCSP-positive B16F10 tumors (C25 line) are infiltrated with a TIL population composed of CD8 memory and effector cells that could potentially recognize the tumor-targeted allogeneic MHC molecule, allowing the tumor-targeted allogeneic MHC molecule to allogeneically stimulate the TCR of CD8+ cells without providing co-stimulation, depending upon already activated cells that could respond and kill tumors, i.e. effector or memory CTLs. In order to establish the presence of such activated tumor infiltrating lymphocytes in the B16F10 tumors a tumor single cell suspension was prepared as above, and stained with CD44 and CD62L to differentiate between Naïve (CD44 low, CD62L+), Effector (CD44 low, CD62L−), Effector Memory (CD44 high, CD62L−) and Central Memory (CD44 high, CD62L+) T cells that are CD8 or CD4 positive. In order to properly identify CD62L+ cells in flow cytometry and to position the gate of the populations, Naïve T cells (CD44 low, CD62L+) were used that were harvested from the spleen of a naïve mouse and analyzed. The dot plot in the bottom left of FIG. 8A shows the stained splenocyte sample and illustrates the gating of CD8 and CD4 (blue and pink respectively) and the CD44 vs CD62L (FIG. 8A, bottom right) gating of the different populations. The top two dot plots of FIG. 8A show the same gates but of a B16F10-MCSP (C25) tumor sample. As expected, the majority of TILs are of effector and memory phenotypes. When the frequencies of the different populations in B16F10-MCSP (C25) were compared to those of the WT B16F10 tumor TILs, no significant differences were found (FIG. 8B).

### Example 8

### MCSP Positive B16F10 Tumor Bearing Mice Treated with the Allogeneic Peptide-H-2Kd-Anti-MCSP scFv Exhibited Significant Inhibition and/or Regression of Tumor Growth when Compared with Mock Treated and Peptide-H-2Kd Treated Mice

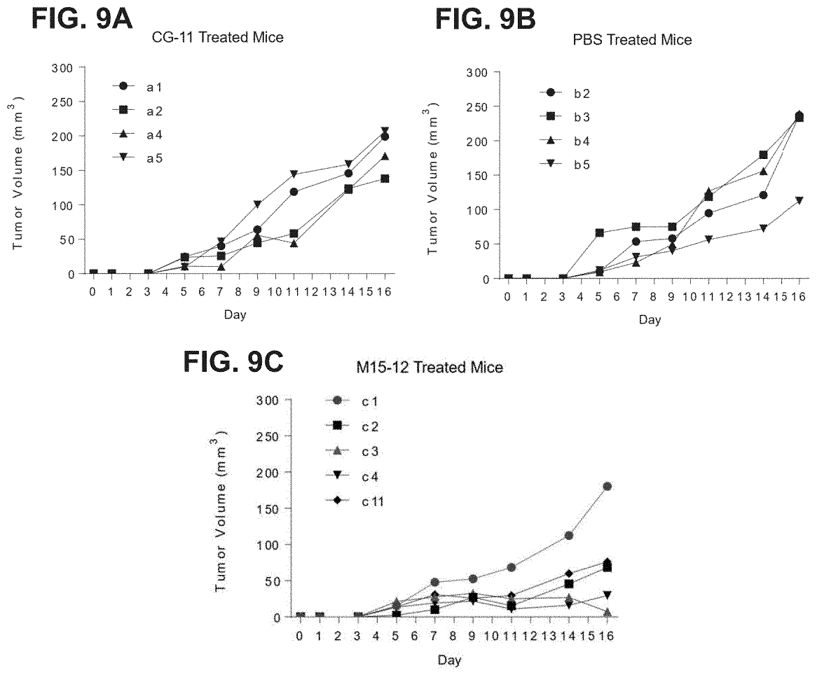

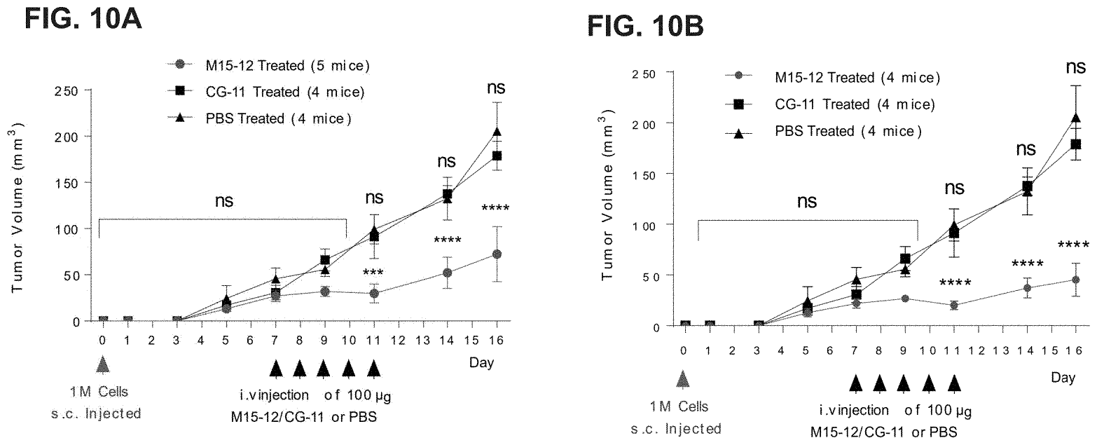

For the first in-vivo experiment, 15 mice were inoculated with 1×10⁶ C25 melanoma cells (MCSP-positive B16F10) in 100 ul PBS. The results of one preliminary experiment are presented in FIGS. 9A-9C. Each plot shows the change in MCSP positive tumor volume (in mm³) of each group of mice treated with PBS, CG-11 (MHC alone) or M15-12 (anti-MCSP-MHC fusion), each line representing a single mouse. When the mice were treated with the syngeneic molecule, M15-747, tumor growth was not significantly different from the PBS treated control mice (data not shown). Tumor diameter (length and width) was measured on the indicated days; the tumors were palpable starting from day 5 and on day 7 the volume was between 25 to 50 mm³. It was determined that day 7 tumors were large enough to start the treatment. Each mouse was treated once per day for five days, receiving a 200 ul tail vain (i.v.) injection of PBS (FIG. 9B), 0.5 mg/ml CG-11 complex (FIG. 9A) or M15-12 molecule (total of 100 ug protein per injection) (FIG. 9C) in PBS. Of the five M15-12 treated mice (FIG. 9C) most of the mice had a negligible tumor volume increase during the treatment phase. Importantly, one mouse (c3, blue triangles in FIG. 9C) rejected the tumor completely, while another mouse (c1, red circles, FIG. 9C) did not respond to the treatment as strongly as the other mice. FIG. 10A summarizes the average tumor volumes (with Standard Error bars) and illustrates that the M15-12 allogeneic H-2Kd/LYQNVGTYV molecule-treated mice had significantly smaller tumors compared to the PBS treated group, starting from the last day of treatment (day 11) and onwards. The statistical significance (P-value) of the observed difference in day 11, was slightly improved (P value reduced) when the non-responsive mouse (c1) was excluded from the analysis (FIG. 10B). Moreover, the CG-11 allogeneic H-2Kd/LYQNVGTYV complex-treated mice did not differ in tumor volume from the PBS-treated group, indicating that the tumor growth inhibition effect of the M15-12 treatment stems from the molecule's MCSP binding activity. Thus, these data suggest significant anti-tumor activity mediated by the Antibody-allogeneic MHC fusion molecule through a T cell engager mode of action that targets allogeneic T cells to the tumor site and induces site-specific allogeneic tumor rejection.

### Example 9

### Mice Treated with the Allogeneic Peptide-H-2Kd-Anti-MCSP scFv but not the Peptide-H-2Kd Complex Mount a B Cell Immune Response and Generate Antibodies Against the H-2Kd Complex

The fundamental concept of the present allogeneic antibody-MHC fusion suggests that allogeneic H-2Kd complexes are immunologically foreign to C57BL/6 mice (H-2Kb). Assessing the serum antibody response is important, because the type of antibodies produced may have positive or negative effects on tumor growth inhibition. Antibodies that recognize the anti-MCSP scFv part of the molecule could prevent tumor binding by the allo-molecule. Antibodies that bind the peptide or MHC groove and block potential TCR-MHC interaction could prevent the hypothesized CTL tumor targeting activity of the allo-molecule. However, antibodies that bind the allo-MHC part of the molecule and can elicit Antibody Mediated Cell-mediated Cytotoxicity (ADCC) via their Fc domain could theoretically cause tumor growth inhibition by ADCC. On the other hand, serum antibodies are expected to bind i.v.-injected allo-molecules before they get to bind cancer cells, forming immune-complexes that can neutralize and prevent the therapeutic benefit of the allo-molecule.

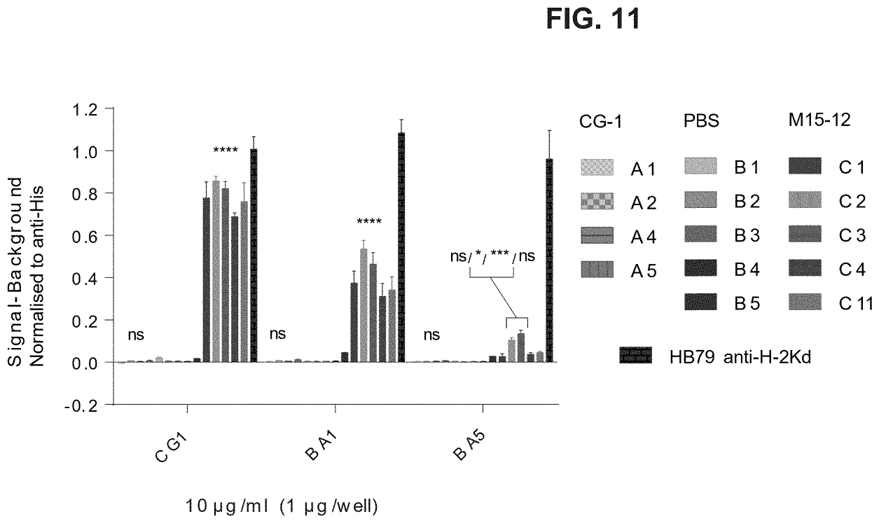

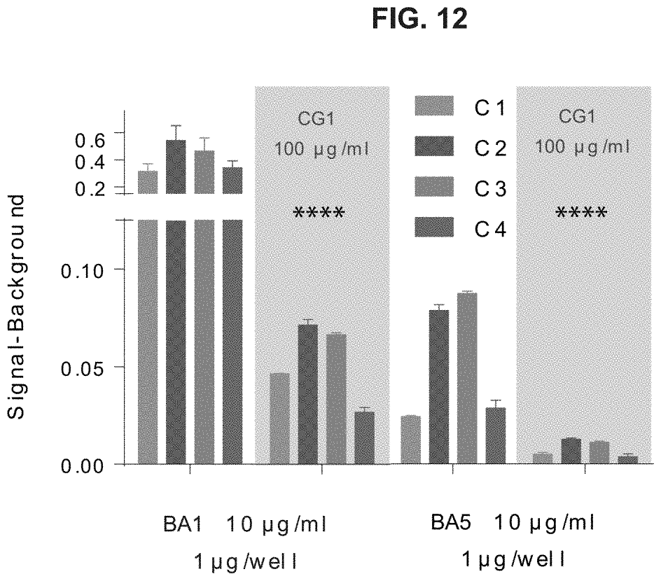

The serum antibody response against the allogeneic molecule (allo-molecule) in treated and control mice was evaluated. On day 16 of the in-vivo experiment described herein (FIGS. 9A-9C and 10A and 10B) mice bearing B16F10 tumors treated with either allo-MHC complex (CG-11), biotinylated allogeneic MHC-anti MCSP (BA-1) or biotinylated syngeneic-MHC anti-MCSP molecules (BA-5) were sacrificed and blood serum was harvested and used in an ELISA assay. Streptavidin coated plates were coated with biotinylated allo-geneic or Syngeneic-MHC anti-MCSP molecules (BA-1 or BA-5 respectively) and allo-MHC complex (CG1) and incubated with diluted serum from treated mice (FIG. 11). The serum of the allo-MHC complex (CG-1, clone 1) and the PBS treated mice did not react with the coated plates, and only M15-1, clone 2 treated mice generated a significant antibody response against the allo-MHC molecule. The response was almost absent when the serum was incubated in syngeneic (BA-5, H-2Kb) molecule coated plates, suggesting that the antibody response is mostly directed against parts of the molecule present in BA-1 (MHC H-2Kd) but absent in BA-5 (MHC H-2Kb), specifically the peptide-MHC part and not the anti-MCSP scFv. Moreover, when a high concentration of unbiotinylated CG-1 (lacking anti-MCSP scFv) complex was added to the diluted serum (FIG. 12) during incubation in BA-1 or BA-5 coated plates, this significantly reduced the signal in both cases. The fact that blocking with CG-1 complex inhibited the signal in BA-5 (MHC H-2Kb) coated plates indicates that the low signal observed is due to antibodies directed against the peptide-MHC part of the molecule that is shared between the syngeneic and allogeneic peptide-MHC complexes (His Tag, connectors and linkers). Without wishing to be bound by a particular hypothesis, these data support the conclusion that the antibody response observed in the treated mice is directed against the peptide-MHC part of the molecule, and that most, but not all of this antibody response is allogeneic-MHC specific.

### Example 10

### Ex-Vivo Experimental System for Testing Human Targeted Allogeneic Rejection Alleles by CD19 Targeted Allo-TCE

An ex-vivo experimental system for testing of the ability of different allo-HLA molecules to initiate CTL dependent allo-rejection of autologous target cells is used to determine correlations between the recipient genotype and the sequences of allo-molecules, in order to generate a decision-tree for identifying optimal fusion protein molecules for each patient.

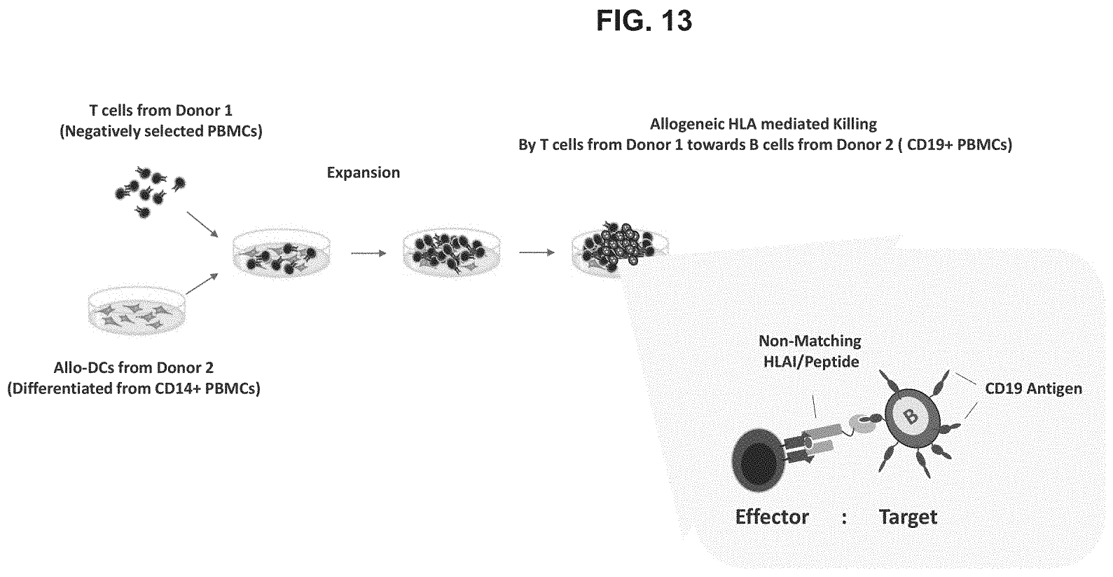

The system is illustrated in FIG. 13: The effector cells are derived from negatively selected T cells obtained from donor 1. The antigen presenting cells (APCs) are positively selected from donor 2, and are derived from CD14+ allo-PBMCs differentiated into mature dendritic cells [e.g. using IL-4 and GMCSF and subsequently activated using a TLR agonist (such as LPS)]. Mature APCs from donor 2 are used to stimulate the allogeneic T cells of donor 1. Following stimulation, sorting of the allogeneic T cells by tetramer staining is performed, followed by in-vitro expansion of the T cells. Target cells are positively selected CD19+ PBMC-derived B cells from donor 1; importantly these cells are obtained from the same donor that donated the effector T cells.

In each experiment, there are one therapeutic allogeneic (HLA mismatched to the T cells) fusion molecule and one control autologous (HLA matched to the T cells) molecule. The fusion molecules comprise an anti-CD19 targeting single chain antibody fragment connected to a peptide-Allo (mismatched) or control, Auto (matched)-HLA molecule (according to donor 1 and 2 HLA genetic makeup). The control autologous molecule is essential for determining the background activity in functional assays, such as direct killing, cytokine secretion, and T cell activation markers. Performing these experiments on PBMCs from donors with different degrees of sequence identity compared to the therapeutic allo-molecule, can enable determination of the optimal correlations between the sequence diversity/polymorphism and the optimal allogeneic T cell functional parameters measured.

### Example 10a

### Ex-Vivo Experimental System for Testing Human Targeted Allogeneic Rejection Alleles by allo-HLA Expressing Autologous Cells

The system is similar to the one illustrated in FIG. 13, but doesn't require the second donor or the manufacturing of allo-molecules: The effector cells are derived from negatively selected T cells obtained from donor 1. The cells can be activated by anti-CD3 antibodies or used immediately for the experiment. Following stimulation and expansion, the activated T cells are coated with capture antibodies specific for INF-gamma and incubated with B cells from donor 1 that were electroporated with RNA coding for an allogeneic HLA allele. The allogeneic cells that recognize the allo-HLA transfected autologous B cells secrete INF-gamma and thus become coated with the cytokine. The coated cells are stained with a fluorophore-conjugated anti-INF-gamma antibody and the allo-T cells are sorted using FACS Aria, followed by in-vitro expansion of the selected T cells. Target cells are positively selected CD19+ PBMC-derived B cells from donor 1; importantly these cells are obtained from the same donor that donated the effector T cells.

In each experiment, there can be more than one allogeneic (HLA mismatched to the T cells) HLA-expressing B cell and one control autologous (HLA matched to the T cells) HLA-expressing cells. The control autologous molecule is essential for determining the background activity in functional assays, such as direct killing, cytokine secretion, and T cell activation markers. Performing these experiments on PBMCs from donors with different HLA sequence identity compared to various therapeutic allo-HLA allele, can enable determination of the optimal correlations between the sequence diversity/polymorphism and the optimal allogeneic T cell functional parameters measured.

### Example 11

### Anti-Fusion Protein Antibodies—Beneficial or Problematic?

Anti-fusion protein antibodies, such as those described in Example 9 may ostensibly enhance treatment using the fusion proteins of the invention by inducing ADCC or inhibit it through the formation of immunological complexes. To address this question, a second round of allogeneic fusion protein treatment is administered to mice that have already mounted a discernible antibody response against the allo-fusion protein molecule. If the second round proves unsuccessful in inducing tumor cell killing in the mice, a follow-up experiment is performed, administering a fusion protein with an H-2Kd MHC class I allele in the first round of treatment, and, once anti-fusion protein molecule antibodies have been detected, administering a fusion protein with an H-2Kk MHC class I allele for the second round. Effective tumor cell targeting and killing in the second round of treatment (using the H-2Kk allele) indicates that the anti-allo-MHC specific antibodies can prevent therapeutic benefit in-vivo, since the neutralizing antibodies from the first round did not inhibit tumor cell killing when a different allele was used for the second treatment cycle. If successful in overcoming inhibition by anti-fusion protein antibodies, subsequent cycles of administration, combined with changing of the alleles can be a possible solution for applying tumor targeted allogeneic rejection strategy in human patients.

Immune Cell Depletion Experiments:

In order to further demonstrate the involvement of B-cells as a potential enhancer or potential inhibitor of allogeneic targeted tumor cell killing, antigen-positive (e.g. MCSP-positive) tumor bearing mice are depleted of their B-cell fraction prior to treatment with an allo-MHC fusion protein. Enhancement of efficacy of the fusion protein on tumor growth with B-cell depletion indicates an inhibitory effect of the anti-fusion protein antibodies, while reduction in the effect on tumor growth in B-cell depleted mice indicates a possible augmentation of the tumor cell killing exerted by the presence of the anti-fusion protein antibodies.

Depletion experiments for other types of immune cells (CD8, CD4 and NK lymphocytes) can also be carried out to determine the critical immune cell population that exert the antibody-targeted allo-rejection of the tumor in-vivo.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Recited in the Application

1. Novak, H. et al. Selective antibody-mediated targeting of class I MHC to EGFR-expres sing tumor cells induces potent antitumor CTL activity in vitro and in vivo. International journal of cancer. Journal International du Cancer 120, 329-36 (2007).
2. Noy, R., Haus-Cohen, M., Oved, K., Voloshin, T. & Reiter, Y. Recruitment of Oligoclonal Viral-Specific T cells to Kill Human Tumor Cells Using Single-Chain Antibody-Peptide-HLA Fusion Molecules. Molecular Cancer Therapeutics 14, 1327-35 (2015).
3. Yang, H., Dundon, P. L., Nahill, R. & Welsh, R. M. VIRUS-INDUCED POLYCLONAL CYTOTOXIC T LYMPHOCYTE STIMULATION. The Journal of Immunology 142, 1710-1718 (1989).
4. Brehm, M. a. et al. Direct Visualization of Cross-Reactive Effector and Memory Allo-Specific CD8 T Cells Generated in Response to Viral Infections. The Journal of Immunology 170, 4077-4086 (2003).
5. Auphan-anezin, N. et al. Distinct orientation of the alloreactive monoclonal CD8 T cell activation program by three different peptide/MHC complexes. European Journal of Immunology 36, 1856-1866 (2006).
6. Selin, L. K. & Brehm, M. a Frontiers in nephrology: heterologous immunity, T cell cross-reactivity, and alloreactivity. Journal of the American Society of Nephrology: JASN 18, 2268-77 (2007).
7. Orsogna, L. J. D., Nguyen, T. H. O., Claas, F. H. J., Witt, C. & Mifsud, N. A. Endogenous-peptide-dependent alloreactivity: new scientific. Tissue Antigens 81, 399-407 (2013).
8. Taylor, P. A. et al. Preformed antibody, not primed T cells, is the initial and major barrier to bone marrow engraftment in allosensitized recipients. Transplantation 109, 1307-1315 (2007).
9. Ciurea, S. O., Lima, M. De, Cano, P., Korbling, M. & Giralt, S. High risk of graft failure in patients with anti-HLA antibodies undergoing haploidentical stem-cell transplantation. Transplantation 88, 1019-1024 (2009).
10. Obst, B. R., Netuschil, N., Klopfer, K., Rammensee, H. & Stevanovi, S. The Role of Peptides in T Cell Alloreactivity Is Determined by Self—Major Histocompatibility Complex Molecules. The Journal of Experimental Medicine 191, 805-812 (2000).
11. Yee, C. et al. Adoptive T cell therapy using antigen-specific CD8%. T cell clones for the treatment of patients with metastatic melanoma: In vivo persistence, migration, and antitumor effect of transferred T cells. Proceedings of the National Academy of Sciences of the United States of America 99, 16168-16173 (2002).
12. Wallen, H. et al. Fludarabine Modulates Immune Response and Extends In Vivo Survival of Adoptively Transferred CD8 T Cells in Patients with Metastatic Melanoma. PloS One 4, (2009).
13. Gross, G., Waks, T. & Eshhar, Z. Expression of immunoglobulin-T-cell receptor chimeric molecules as functional receptors with antibody-type specificity. Proceedings of the National Academy of Sciences of the United States of America 86, 10024-10028 (1989).
14. Goverman, J., Gomez, S. M., Segesman, K. D. & Hunkapiller, T. Chimeric Immunoglobulin-T Cell Receptor Proteins Form Functional Receptors: Implications for T Cell Receptor Complex Formation and Activation. Cell 60, 929-939 (1990).
15. Gill, S., June, C. H. & June, C. H. Going viral: chimeric antigen receptor T-cell therapy for hematological malignancies. Immunological reviews 263, 68-89 (2015).
16. Maude, S. L. et al. Chimeric Antigen Receptor T Cells for Sustained Remissions in Leukemia. New England Journal of Medicine 371, 1507-1517 (2015).
17. Porter, D. L., Levine, B. L., Kalos, M., Bagg, A. & June, C. H. Chimeric Antigen Receptor-Modified T Cells in Chronic Lymphoid Leukemia. New England Journal of Medicine 365, 725-733 (2012).
18. Grupp, S. A. et al. Chimeric Antigen Receptor-Modified T Cells for Acute Lymphoid Leukemia. New England Journal of Medicine 368, 1509-1518 (2014).

What is claimed is:

1. A method of killing a tumor cell presenting a tumor antigen, the method comprising administering to an individual a composition-of-matter comprising at least one fusion protein comprising a viral MHC-restricted peptide; a human beta-2-microglobulin; an alpha chain of a human MHC class I molecule and a binding domain of an antibody which specifically binds to said tumor antigen, wherein said alpha chain of a human MHC molecule is allogeneic to the individual, so as to elicit an alloimmune response to said tumor cell presenting said antigen, thereby killing said tumor cell.

2. The method of claim 1, wherein:
(i) said alpha chain of said human MHC class I molecule is an extracellular portion of said alpha chain of said human MHC class I, comprising the human extracellular alpha1, alpha 2 and alpha 3 MHC class I domains, and/or
(ii) wherein said viral MHC-restricted peptide, said human beta-2-microglobulin; said alpha chain of said human MHC class I molecule and said binding domain of an antibody which specifically binds to said tumor antigen are N-terminally to C-terminally respectively sequentially translationally fused.

3. The method of claim 1, wherein:
(i) said viral MHC-restricted peptide and said human beta-2-microglobulin are connected by a first peptide linker having an amino acid sequence about 15 amino acids in length, and/or
(ii) said human beta-2-microglobulin and said alpha chain of a human MHC class I molecule are connected via a second peptide linker having an amino acid sequence about 20 amino acids in length, and/or
(iii) wherein said alpha chain of said human MHC class I molecule and said binding domain of said antibody which specifically binds to said tumor antigen are connected via a third peptide linker having the amino acid sequence ASGG;
wherein the amino acid sequence of said first peptide linker can be GGGGSGGGGSGGGGS (SEQ ID NO: 16) and
the amino acid sequence of said second peptide linker can be GGGGSGGGGSGGGGSGGGGS (SEQ ID NO: 18).

4. The method of claim 1, wherein said binding domain of said antibody which specifically binds to said tumor antigen is a ScFv fragment of said antibody.

5. The method of claim 1, wherein said composition of matter comprises a plurality of said fusion proteins having different allogeneic human MHC molecule alpha chains, and/or wherein the amino acid sequence of said alpha chain of said human MHC class I molecule is no more than 95% identical compared to the amino acid sequences of both of the HLA class I α1-α2 alleles of the individual.

6. The method of claim 1, further comprising determining the MHC class I type of said individual prior to said administering.

7. The method of claim 6, comprising selecting said human MHC molecule alpha chain of said fusion protein based on the MHC class I type of said individual as determined prior to said administering.

8. The method of claim 1, wherein:
(i) said tumor cell presents mesothelin on its surface and, optionally, said binding domain of said antibody specifically binds to mesothelin, or
(ii) wherein said tumor cell presents MCSP on its surface, and optionally wherein said binding domain of said antibody specifically binds to MCSP.

9. The method of claim 1, comprising repeating said administering said composition of matter.

10. The method of claim 1, comprising a plurality of successive cycles of administration, wherein each cycle of administration comprises administering a composition of matter comprising at least one fusion protein comprising a viral MHC-restricted peptide; a human beta-2-microglobulin; an alpha chain of a human MHC class I molecule and a binding domain of an antibody which specifically binds to said tumor antigen, wherein said alpha chain of a human MHC class I molecule is allogeneic to the individual and wherein said alpha chain of said human MHC class I molecule is non-identical to the alpha chain of said human MHC class I molecule of previous cycles of administration, and, optionally, wherein said cycles of administration are separated by intervals of at least 1 week.

11. The method of claim 9, further comprising assessing said alloimmune response to said tumor cell in said individual, and commencing a new cycle of administration upon detecting reduced alloimmune response to said alpha chain of said human MHC class I molecule.

12. A composition-of-matter comprising a plurality of fusion proteins, each fusion protein comprising a viral MHC-restricted peptide; a human beta-2-microglobulin; an alpha chain of a human MHC class I molecule and a binding domain of an antibody which specifically binds to a tumor antigen, wherein said plurality of fusion proteins comprises:
(i) at least two non-identical fusion proteins having different allogeneic human MHC class I molecule alpha chains, or
(ii) at least two non-identical fusion proteins having different viral MHC-restricted peptides, or
(iii) at least two non-identical fusion proteins having a different binding domain of an antibody which specifically binds to a tumor antigen.

13. An article of manufacture comprising a plurality of fusion proteins each packaged in a different package, each fusion protein comprising a viral MHC-restricted peptide; a human beta-2-microglobulin; an alpha chain of a human MHC class I molecule and a binding domain of an antibody which specifically binds to a tumor antigen, wherein said plurality of fusion proteins comprises:
(i) at least two non-identical fusion proteins having different allogeneic human MHC class I molecule alpha chains, or
(ii) at least two non-identical fusion proteins having different viral MHC-restricted peptides, or
(iii) at least two non-identical fusion proteins having a different binding domain of an antibody which specifically binds to a tumor antigen.

14. The composition of matter of claim 12, wherein said alpha chain of said non-identical human MHC class I molecules are selected from the group consisting of HLA-A23, HLA-A32, HLA-A74, HLA-A31, HLA-A80, HLA-A36, HLA-A25, HLA-A26, HLA-A43, HLA-A34, HLA-A66, HLA-A69, HLA-A68, HLA-A29, HLA-B14, HLA-B18, HLA-B27, HLA-B38, HLA-B39, HLA-B41, HLA-B42, HLA-B47, HLA-B48, HLA-B49, HLA-B50, HLA-B52, HLA-B53, HLA-B54, HLA-B55, HLA-B56, HLA-B57, HLA-B58, HLA-B59, HLA-B67, HLA-B73, HLA-B78, HLA-B82, HLA-B81.

15. The composition of matter of claim 12, wherein said alpha chain of said non-identical human MHC class I molecule has an amino acid sequence at least 95% identical to an amino acid sequence selected from the group consisting of HLA-A23:01:01 (SEQ ID NO: 44), HLA-A32:01:01 (SEQ ID NO: 47), HLA-A74:01:01 (SEQ ID NO: 55), HLA-A31:01:02 (SEQ ID NO: 57), HLA-A80:01:01 (SEQ ID NO: 49), HLA-A36:01 (SEQ ID NO: 56), HLA-A25:01:01 (SEQ ID NO: 45), HLA-A26:01:01(SEQ ID NO: 52), HLA-A43:01(SEQ ID NO: 53), HLA-A34:01:01(SEQ ID NO: 48), HLA-A66:01:01(SEQ ID NO: 50), HLA-A69:01:01(SEQ ID NO: 51), HLA-A68:01:01(SEQ ID NO: 54), HLA-A29:01:01(SEQ ID NO: 46), HLA-B14:01:01(SEQ ID NO: 58), HLA-B18:01:01(SEQ ID NO: 59), HLA-B27:02:01(SEQ ID NO: 60), HLA-B38:01:01(SEQ ID NO: 61), HLA-B39:01:01(SEQ ID NO: 62), HLA-B41:01:01(SEQ ID NO: 63), HLA-B42:01:01(SEQ ID NO: 64), HLA-B47:01:01(SEQ ID NO: 65), HLA-B48:01:01(SEQ ID NO: 66), HLA-B49:01:01(SEQ ID NO: 67), HLA-B50:01:01(SEQ ID NO: 68), HLA-B52:01:01(SEQ ID NO: 69), HLA-B53:01:01(SEQ ID NO: 70), HLA-B54:01:01(SEQ ID NO: 71), HLA-B55:01:01(SEQ ID NO: 72), HLA-B56:01:01(SEQ ID NO: 73), HLA-B57:01:01(SEQ ID NO: 74), HLA-B58:01:01(SEQ ID NO: 75), HLA-B59:01:01(SEQ ID NO: 76), HLA-B67:01:01(SEQ ID NO: 77), HLA-B73:01(SEQ ID NO: 78), HLA-B78:01:01(SEQ ID NO: 79), HLA-B82:01(SEQ ID NO: 80), HLA-B81:01 (SEQ ID NO: 81).

16. The composition of matter of claim 12, wherein said plurality of fusion proteins comprises at least two non-identical fusion proteins having different viral MHC-restricted peptides and optionally, wherein said viral MHC-restricted peptide is 8 or 9 amino acids in length.

17. The composition of matter of claim 12, wherein said plurality of fusion proteins comprises at least two non-identical fusion proteins having a different binding domain of an antibody which specifically binds to a tumor antigen and wherein said binding domain of said antibody specifically binds to a tumor antigen selected from the group consisting of mesothelin, MCSP and CD25 receptor.

18. The composition of matter of claim 12, wherein said binding domain of an antibody which specifically binds to MCSP has an amino acid sequence comprising SEQ ID NO: 27.

19. The composition of matter of claim 12 wherein said alpha chain of said human MHC class I molecule is an extracellular portion of said alpha chain of said human MHC class I, comprising the human extracellular alpha1, alpha 2 and alpha 3 MHC class I domains.

20. An assay for identifying allogeneic human MHC class I alpha chains effective for eliciting an alloimmune response in a subject, the assay comprising:
i) contacting PBMC-derived T cells from said subject with antigen presenting cells from a donor mismatched for MHC class I, thereby activating said T cells;
ii) isolating and culturing said T cells;
iii) contacting said T-cells with
a) a CD19+ B-cell target cell of said subject, and
b) a fusion protein comprising a viral MHC-restricted peptide; a human beta-2-microglobulin; an alpha chain of a human MHC class I molecule HLA-mismatched for said subject and a binding domain of an antibody which specifically binds CD19, and
iv) assaying an immune response of said B-cells,
v) repeating steps i)-iv) using an autologous fusion protein comprising said viral MHC-restricted peptide; said human beta-2-microglobulin and an alpha chain of a human MHC class I molecule HLA-matched for said subject, and
vi) determining effectiveness of said allogeneic human MHC class I alpha chain for eliciting an alloimmune response in said subject by comparing said an immune response of said B-cells of said allogeneic with that of said autologous fusion protein, wherein said immune response of said B cells is selected from the group consisting of direct killing of said B-cells, cytokine secretion and T cell activation markers.

21. The assay of claim 20, wherein said alpha chain of said human MHC class I molecule is an extracellular portion of said alpha chain of said human MHC class I, comprising the human extracellular alpha1, alpha 2 and alpha 3 MHC class I domains.

22. The method of claim 1 wherein said alpha chain of a human MHC class I molecule is HLA-A 34.

23. The composition of matter of claim 12 wherein said alpha chain of a human MHC class I molecule is HLA-A 34.

24. The article of manufacture of claim 13 wherein said alpha chain of a human MHC class I molecule is HLA-A 34.
